(19) United States
(12) Patent Application Publication
(10) Pub. No.: US 2006/0172294 A1
(43) Pub. Date: Aug. 3, 2006

(54) Detection of epigenetic abnormalities and diagnostic method based thereon

(76) Inventor: Arturas Petronis, Toronto (CA)

Correspondence Address: Armstrong Kratz Quintos Hanson & Brooks;Intellectual Property Law Offices, 502 Washington Avenue, Suite 220, Towson, MD, 21204, US

(21) Appl. No.: 10/516,406

(22) PCT Filed: Jun. 6, 2003
(86) PCT No.: PCT/CA03/00820

§ 371 Date: Dec. 1, 2004

Related U.S. Application Data
(60) Provisional application No. 60/386,818, filed on Jun. 6, 2002.

(51) Int. Cl. C12Q 1/68

(52) U.S. Cl. 435/6

(57) ABSTRACT

The present invention provides a method of detecting an epigenetic abnormality associated with a disease. The method comprises identifying, within a eukaryotic genome, a locus having a hypomethylated sequence specific for the disease and an endogenous multi-copy DNA element. The method can also comprise separate steps of identifying a disease-specific hypomethylated sequence and identifying an endogenous multi-copy DNA element, where the steps may be performed in any order, so long as a locus is identified that has both a disease-specific hypomethylated sequence and an endogenous multi-copy DNA element. The disease-specific hypomethylated sequences detected in accordance with the present invention indicate putative regions of epigenetic dys-regulation and indicate aberrantly regulated nucleic acid sequences that may cause or predispose a patient to disease, such as, but not limited to, Huntingdon s disease, cancers, diabetes, schizophrenia, or bipolar disorder.

The present invention relates to identification of epigenetic abnormalities. More particularly, the present invention relates to diagnosis of diseases based on DNA methylation differences, and identification and isolation of genes that cause such diseases.

### BACKGROUND OF THE INVENTION

Substantial progress has been made in recent years with respect to the diagnosis and treatment of diseases in which a single defective gene is responsible. Traditional linkage studies have effectively isolated the causal gene and allowed for the further development of diagnostic tests and furthered research into treatments such as gene therapy for conditions such as cystic fibrosis, Duchennes muscular dystrophy, Huntington's disease and fragile X syndrome. However, similar progress has not been made in diseases caused by mutations in multiple genes. Traditional linkage studies in complex diseases such as schizophrenia, bipolar disorder, cancers and diabetes have only succeeded in isolating chromosome regions, often containing 200-300 genes. The ability to screen such a large number of genes is clearly a time-consuming and daunting task.

Epigenetic mechanisms can be an important factor in complex, multi-factorial diseases such as cancers. Epigenetics refers to modifications in gene expression that are brought about by heritable, but potentially reversible changes in DNA methylation and chromatin structure (Henikoff S, Matzke M A Exploring and explaining epigenetic effects. Trends Genet 1997,13(8):293-5; Siegfried Z, Eden S, Mendelsohn M, Feng X, Tsuberi B Z, Cedar H. DNA methylation represses transcription in vivo. Nat Genet 1999, 22(2):203-206; Gonzalgo, M. L. and Jones, P. A. (1997) Mutagenic and epigenetic effects of DNA methylation. Mutat. Res. 386(2), 107-18; Razin, A. and Shemer, R. (1999) Epigenetic control of gene expression. Results Probl. Cell. Differ. 25, 189-204; Lyko, F. and Paro, R. (1999) Chromosomal elements conferring epigenetic inheritance. Bioessays 21(10), 824-32). DNA methylation of the binding sites for transcription factors changes the affinity of such factors for regulatory sequences, which affects the transcriptional activity of a gene (Ehrlich M and Ehrlich K (1993) Effect of DNA methylation and the binding of vertebrate and plant proteins to DNA. In: Jost J P and Saluz P (eds) DNA Methylation: Molecular Biology and Biological Significance pp. 145-168. Birkhauser Verlag, Basel, Switzerland; Riggs A, Xiong Z, Wang L, and LeBon J M (1998) Methylation dynamics, epigenetic fidelity and X chromosome structure. In: Wolffe A P (ed) Epigenetics, pp. 214-227. John Wiley & Sons, Chistester). In addition to positional effects of methylated cytosines, density in a gene regulatory region also contributes to gene activity. This type of regulation is mediated by methylated cytosine binding proteins and acetylation of histones (Jones P L, Veenstra G J, Wade P A, Vermaak D, Kass S U, Landsberger N, Strouboulis J, and Wolffe A P (1998) Methylated DNA and MeCP2 recruit histone deacetylase to repress transcription. Nature Genetics 19: 187-91; Nan X, Ng H H, Johnson C A, Laherty C D, Turner B M, Eisenman R N, and Bird A (1998). Transcriptional repression by the methyl-CpG-binding protein MeCP2 involves a histone deacetylase complex. Nature 393: 386-9; Robertson K D and Wolffe A P (2000) DNA methylation in health and disease. Nature Review Genet 1:11-9).

Methylation can occur within cytosine-guanosine islands (CpG islands) that are typically between 0.2 to about 1 kb in length and are located upstream of many housekeeping and tissue-specific genes, but may also extend into protein coding regions. Methylation of cytosine residues contained within CpG islands of certain genes has been inversely correlated with gene activity. This could lead to decreased gene expression by a variety of mechanisms including, for example, disruption of local chromatin structure, inhibition of transcription factor-DNA binding, or by recruitment of proteins which interact specifically with methylated sequences indirectly preventing transcription factor binding. Some studies have demonstrated an inverse correlation between methylation of CpG islands and gene expression. Tissue-specific genes are usually unmethylated within the receptive target organ cells but are methylated in the germline and in non-expressing adult tissues. CpG islands of constitutively-expressed housekeeping genes are normally unmethylated in the germline and in somatic tissues.

In comparison to the role of DNA hypermethylation in disease, the role of DNA hypomethylation has attracted much less attention from researchers. However, DNA hypomethylation has been generally linked to disease states. For example, cancerous tissue has been shown to have lower levels of DNA methylation when compared to normal tissue (Lapeyre, J. N. and Becker, F. F. (1979). 5-Methylcytosine content of nuclear DNA during chemical hepatocarcinogenesis and in carcinomas which result. Biochem Biophys Res Commun 87, 698-705; Gama-Sosa, M. A., Slagel, V. A., Trewyn, R. W., Oxenhandler, R., Kuo, K. C., Gehrke, C. W., and Ehrlich, M. (1983). The 5-methylcytosine content of DNA from human tumors. Nucleic Acids Res 11, 6883-94; Feinberg, A. P., Gehrke, C. W., Kuo, K. C., and Ehrlich, M. (1988). Reduced genomic 5-methylcytosine content in human colonic neoplasia. Cancer Res 48, 1159-61). Furthermore, activation of oncogenes as a result of DNA hypomethylation has been proposed (Feinberg, A. P. and Vogelstein, B. (1983) Hypomethylation of ras oncogenes in primary human cancers. Biochem Biophys Res Commun 111, 47-54). Although a significant correlation between DNA hypomethylation and diseased states has been established, there is a need for methodology for identifying specific DNA hypomethylation-based epigenetic abnormalities that may increase the risk of developing a diseased state.

U.S. Pat. No. 5,871,917 discloses methods for detecting epigenetic abnormalities comprising: restriction of genomic DNA with a methylation-sensitive restriction enzyme (a restriction enzyme that cleaves an unmethylated site, but does not cleave the same site if it is methylated) that leaves an overhang; ligation of adaptors to the overhangs; PCR amplification with primers directed to the adaptors; followed by a subtractive hybridization to eliminate house keeping genes; and a second round of PCR amplification with a second set of primers directed to a second set of adaptors. A problem with this design is that the method is limited to a restriction enzyme that leaves overhangs and, further, the method is complicated due to the ligation of two sets of adaptors.

WO99/01580 discloses methods for detection of genomic imprinting disorders based on digestion of genomic DNA with methylation-sensitive restriction enzymes and PCR amplification using primers. One embodiment, directed to the detection of unmethylated sequences, requires the use of a restriction enzyme that leaves overhangs and the use of exogenous adaptors, and therefore suffers from similar disadvantages as those described above in regards to U.S. Pat. No. 5,871,917. Another embodiment, directed to the detection of methylated sequences, uses primers directed to endogenous elements such that exogenous adaptors are not required, but these primers are required to be positioned on either side of a methylation-sensitive restriction site. Since a methylation sensitive restriction enzyme will cut an unmethylated site, this method can only be used to amplify the methylated sequences, and cannot produce an unmethylated sequence which will be cut in between the two primers.

It is an object of the present invention to overcome disadvantages of the prior art.

The above object is met by a combination of the features of the main claims. The sub claims disclose further advantageous embodiments of the invention.

### SUMMARY OF THE INVENTION

The present invention relates to detection of epigenetic abnormalities and diagnosis of diseases associated with epigenetic abnormalities, and identification and isolation of genes that cause such diseases.

According to the present invention there is provided a method of detecting an epigenetic abnormality associated with a disease comprising: identifying, within a eukaryotic genome, a locus having a hypomethylated sequence specific for said disease and an endogenous multi-copy DNA element. The method can comprise separate steps of identifying a disease-specific hypomethylated sequence and identifying an endogenous multi-copy DNA element, where the steps may be performed in any order, so long as a locus is identified that has both a disease-specific hypomethylated sequence and an endogenous multi-copy DNA element. The disease-specific hypomethylated sequence and the endogenous multi-copy DNA element will often be within 20 kilobases of separation, for example, within 20, 10, 5, 2, 1, 0.1 kilobases of each other, or may even be so close as to overlap. The endogenous multi-copy DNA element can include any retroelement that is normally methylated examples of which include, without limitation, endogenous retroviral sequences (ERV), Alu sequences, and LINE sequences. The endogenous multi-copy DNA element may be located within any eukaryotic genome including fungi, plants, and animals, with mammalian and human genomes being non-limiting examples of animal genomes.

In another aspect, the present invention provides a method of identifying a chromosomal region associated with a diseased state comprising: identifying a locus, within DNA obtained from a diseased sample, that has a DNA sequence that is hypomethylated and an endogenous multi-copy DNA element, wherein the DNA sequence is methylated in a non-disease sample and wherein the chromosomal region consists of from about 1 to about 10 DNA coding sequences that are proximal to the identified locus. In a further aspect, a DNA coding sequence having an epigenetically altered expression pattern that contributes to a disease in an organism can be identified by comparing expression patterns of the DNA coding sequence located proximal to the disease-specific hypomethylated locus within a test sample that exhibits characteristics of said disease with expression patterns of a corresponding DNA coding sequence within a control sample to identify the DNA coding sequence having an epigenetically altered expression pattern. The DNA coding sequence may encode an RNA that remains non-translated, or may encode an RNA that is translated, at least partially, into a polypeptide.

In another aspect, the present invention provides a method of diagnosing an epigenetic abnormality correlated with a disease comprising: identifying a DNA sequence that is hypomethylated within a locus that has an endogenous multi-copy DNA element and is obtained from a diseased sample, wherein the DNA sequence is methylated in a non-disease sample.

According to yet another aspect of the present invention there is provided a method of detecting an epigenetic abnormality associated with a disease, the method comprising:

a) extraction of genomic DNA from a sample that exhibits characteristics of a disease;

b) digestion of the genomic DNA with a methylation-sensitive restriction enzyme to produce a pool of restricted DNA fragments;

c) fractionation of the pool of restricted DNA fragments to obtain DNA fragments of a desired size;

d) amplification of at least a segment of the DNA fragments of a desired size with primers that anneal to an endogenous DNA element to produce a PCR product;

e) cloning of the PCR product into a sequencing vector;

f) sequence determination of the PCR product to obtain a sequence of the PCR product;

g) comparing the sequence against a genomic database to assign a locus for the epigenetic abnormality associated with a disease.

The sample from which DNA is extracted may be any cell, tissue, organ or other suitable specimen that exhibits characteristics of a disease. For example, without wishing to be limiting, in an individual suffering from schizophrenia, Huntingdon's disease, or bipolar disorder a sample may be obtained from brain tissue.

Any endogenous multi-copy DNA element that is found to have epigenetic abnormalities associated with a disease can be PCR amplified according to the present invention. In a further aspect, the endogenous DNA element is a multi-copy DNA element. In a still further aspect, the multi-copy DNA element is selected from the group consisting of LINE, SINE, L1, and Alu.

In still another aspect, the present invention provides a method of identifying a gene having an epigenetically altered expression pattern that contributes to a disease in an organism, the method comprising:

a) extraction of genomic DNA from a sample that exhibits characteristics of a disease;

b) digestion of the genomic DNA with a methylation-sensitive restriction enzyme to produce a pool of restricted DNA fragments;

c) fractionation of the pool of restricted DNA fragments to obtain DNA fragments of a desired size;

d) amplification of at least a segment of the DNA fragments of a desired size with primers that anneal to an endogenous DNA element to produce a PCR product;

e) cloning of the PCR product into a sequencing vector;

f) sequence determination of the PCR product to obtain a sequence of the PCR product;

g) comparing the sequence against a genomic database to assign a locus for said epigenetic abnormality associated with a disease;

h) searching said database to identify a gene located proximal to said locus;

i) comparing expression patterns of said gene located proximal to said locus within a test sample that exhibits characteristics of said disease with expression patterns of a corresponding gene within a control sample to identify said gene having an epigenetically altered expression pattern.

Genes can be identified in accordance with the present invention from any eukaryotic organism including, plants and animals, where epigenetic abnormality is associated with the occurrence of disease.

In yet another aspect, the present invention provides a method of isolating a probe for detecting an epigenetic abnormality associated with a disease in an animal, said method comprising:

a) extraction of genomic DNA from a sample that exhibits characteristics of said disease;

b) digestion of said genomic DNA with a methylation-sensitive restriction enzyme to produce a pool of restricted DNA fragments;

c) fractionation of said pool of restricted DNA fragments to obtain DNA fragments of a desired size;

d) amplification of at least a segment of said DNA fragments of a desired size with primers that anneal to an endogenous DNA element to produce a PCR product;

f) using said PCR product as said probe to detect said epigenetic abnormality associated with said disease in another sample.

In still another aspect, there is provided methods for detecting disease or diagnosing disease. In an aspect the present invention provides a method of detecting a disease associated with an epigenetic abnormality comprising, identifying, within a eukaryotic genome, a locus having a hypomethylated sequence specific for the disease and an endogenous multi-copy DNA element. In another aspect the present invention provides a method of diagnosing a disease correlated with an epigenetic abnormality comprising identifying a DNA sequence that is hypomethylated within a locus that has an endogenous multi-copy DNA element and is obtained from a diseased sample, the DNA sequence being methylated in a non-disease sample.

The methods of the present invention can be applied to any disease that occurs as a result of hypomethylation within a locus having an endogenous multi-copy DNA element, including Mendelian and non-Mendelian disease. Illustrative examples of diseases include, without limitation, Huntington's disease, schizophrenia, bipolar disorder, cancers, neuropsychiatric diseases, and diabetes.

This summary does not necessarily describe all necessary features of the invention but that the invention may also reside in a sub-combination of the described features.

### BRIEF DESCRIPTION OF THE DRAWINGS

These and other features of the invention will become more apparent from the following description in which reference is made to the appended drawings wherein:

FIG. 1 shows the localization of the cloned Alu elements.

FIG. 2 shows DNA coding sequences that comprise or are located within very close proximity (within 100,000 bp) of cloned Alu elements.

FIG. 3 shows sequences of cloned Alu elements in Example 4 (SEQ ID NO:29-263).

FIG. 4 shows an alignment of a portion of cloned Alu elements in Example 1 (SEQ ID NO:6-28). Alignment file of cloned Alu sequences was created using CLUSTAL W Multiple Sequencing Alignment Program (http://clustal w.genome.ad.jp/).

### DESCRIPTION OF PREFERRED EMBODIMENT

The invention relates to methods and compositions for identification of epigenetic abnormalities. More particularly, the present invention relates to diagnosis of diseases based on DNA methylation differences and identification of genes that cause such diseases. The present invention provides methods and compositions for detecting and isolating DNA sequences which are abnormally or differentially methylated in a diseased cell type when compared to a normal cell type.

Traditional linkage studies in complex diseases such as schizophrenia, bipolar disorder, cancers and diabetes have only succeeded in isolating chromosome regions, often containing 200-300 genes. The ability to screen such a large number of genes is clearly a time-consuming and daunting task. The present invention provides a short-cut in determining which genes within a 200-300 gene region are in fact responsible for the onset of a major disease such as diabetes, schizophrenia, cancers, or bipolar disorder. According to the present invention differentially modified, endogenous multi-copy DNA elements can act as markers for genes which are dys-regulated. Epigenetic analysis of so called “junk” DNA leads to a ‘short-cut’ in identification of specific genes, dys-regulation of which increases the risk to major disease.

The following description is of a preferred embodiment by way of example only and without limitation to the combination of features necessary for carrying the invention into effect.

The methylation patterns of DNA from tumor cells are generally different than those of normal cells (Laird et al., DNA Methylation and Cancer, 3 Human Molecular Genetics 1487, 1488 (1994)). Tumor cell DNA is generally undermethylated relative to normal cell DNA, but selected regions of the tumor cell genome may be more highly methylated than the same regions of a normal cell's genome. Hence, detection of altered methylation patterns in the DNA of a tissue sample is an indication that the tissue is cancerous. For example, the gene for Insulin-Like Growth Factor 2 (IGF2) is hypomethylated in a number of cancerous tissues, such as Wilm's Tumors, rhabdomyosarcoma, lung cancer and hepatoblastomas (Rainner et al. 362 Nature 747-49 (1993); Ogawa, et al., 362 Nature 749-51 (1993); S. Zhan et al., 94 J. Clin. Invest. 445-48 (1994); P. V. Pedone et al., 3 Hum. Mol. Genet. 1117-21 (1994); H. Suzuld et al., 7 Nature Genet 432-38 (1994); S. Rainier et al., 55 Cancer Res. 1836-38 (1995)).

Alteration of methylation may be a key, and common event, in the development of neoplasia and may play at least two roles in tumorigenesis:

1) DNA hypomethylation may cause an increase in proto-oncogene expression or DNA hypermethylation may decrease expression of a tumor supressor which contributes to neoplastic growth; and

2) DNA hypomethylation may change chromatin structure, and induce abnormalities in chromosome pairing and disjunction. Such structural abnormalities may result in genomic lesions, such as chromosome deletions, amplifications, inversions, mutations, and translocations, all of which are found in human genetic diseases and cancer.

While the present invention can be used for detecting any alteration in methylation, the present invention is particularly useful for detecting and isolating DNA fragments that are normally methylated but which, for some reason, are non-methylated in a proportion of cells. Such DNA fragments may normally be methylated for a number of reasons. For example, such DNA fragments may be normally methylated because they contain, or are associated with, genes that are rarely expressed, genes that are expressed only during early development, genes that are expressed in only certain cell-types, and the like.

As used herein, hypomethylation means that at least one cytosine in a CG or CNG di- or tri-nucleotide site in genomic DNA of a given cell-type does not contain CH₃ at the fifth position of the cytosine base. Cell types that may have hypomethylated CGs or CNGs, such as, without limitation, CCGs, include any cell type that may be expressing a non-housekeeping function. This includes both normal cells that express tissue-specific or cell-type specific genetic functions, as well as tumorous, cancerous, and similar cell types. Cancerous cell types and conditions which can be analyzed, diagnosed or used to obtaining probes by the present methods include, but are not limited to, Wilm's cancer, breast cancer, ovarian cancer, colon cancer, kidney cell cancer, liver cell cancer, lung cancer, leukemia, rhabdomyosarcoma, sarcoma, and hepatoblastoma.

A method of the present invention is directed to detection of an epigenetic abnormality comprising identifying, within a eukaryotic genome, a locus having a hypomethylated sequence and an endogenous multi-copy DNA element. The method can comprise separate steps of identifying a hypomethylated sequence and identifying an endogenous multi-copy DNA element, where the steps may be performed in any order, so long as a locus is identified that has both a hypomethylated sequence and an endogenous multi-copy DNA element. The hypomethylated sequence and the endogenous multi-copy DNA element will often be within 20 kilobases of separation, for example, within 20, 10, 5, 2, 1, 0.1 kilobases of each other, or may even be so close as to overlap. The endogenous multi-copy DNA element can include any retroelement, examples of which include, without limitation, endogenous retroviral sequences (ERV), Alu sequences, L1 sequences, SINE sequence, and LINE sequences. The endogenous multi-copy DNA element will be located within any eukaryotic genome including fungi, plants, and animals, with mammalian and human genomes being non-limiting examples of animal genomes.

Without wishing to be bound by theory, hypermethylation in a locus having a retroelement, within eukaryotic genomes, can function to suppress transcriptional activity of the retroelement. Hypomethylation may underlie disease by undesired removal of the suppression of transcriptional activation of a retroelement and/or surrounding genes. As such the combination of a hypomethylated sequence and a retroelement can serve as a useful marker for an aberrant regulation of DNA sequence expression that can be a factor in a diseased state.

As will be recognized by persons skilled in the art, various techniques may be used to identify a locus having a hypomethylated sequence and an endogenous multi-copy DNA element. For example, techniques that are known to be reliable for detecting differences in DNA methylation include, but are not limited to:

methylation-sensitive restriction enzymes (Issa J. P., et al. (1994) Nature Genetics 7:536-40);

methylation-sensitive arbitrarily primed PCR (Liang G, et al. (2002) Identification of DNA methylation differences during tumorigenesis by methylation-sensitive arbitrarily primed polymerase chain reaction. Methods 27(2):150-5);

sequencing of sodium bisulfite-induced modifications of genomic DNA (Frommer M, et al. (1992) A genomic sequencing protocol that yields a positive display of 5-methylcytosine residues in individual DNA strands);

methylation-specific PCR based on differential hybridization of PCR primer to DNA initially modified by bisulfite treatment (Herman J G, et al. (1996) Methylation-specific PCR: A novel PCR assay for methylation status of CpG islands. Proc Natl Acad Sci USA 93:9821-26; Fan X, et al. (Improvement of the methylation specific PCR technical conditions for the detection of p16 promoter hypermethylation in small amounts of tumor DNA. Oncology Rep 9:181-3); or

methylation-sensitive single nucleotide primer extension based on bisulfite-modification of DNA followed by differential incorporation of labelled nucleotides to a primer that is designed to hybridise immediately upstream of a methylation site (Gonzalgo and Jones (1997) Rapid quantitation of methylation differences at specific sites using methylation-sensitive single nucleotide primer extension (Ms-SNuPe) Nucleic Acids Research 25:2529-31).

Several techniques are also available for identifying an endogenous multi-copy DNA element within a locus. For example, endogenous multi-copy DNA elements can be localized in silico for genomes that have been sequenced, annotated and deposited within public, private, or commercial databases. As another example, PCR primers can be used to detect the presence of an endogenous multi-copy DNA element within a larger DNA sequence. As yet another example, Southern hybridisation with probes comprising an endogenous multi-copy DNA element sequence can be used for identifying and localizing the presence of the multi-copy DNA element within a larger DNA sequence.

Hypomethylation of genomic sequences can be determined by using both methylation-sensitive restriction enzyme analysis, and genomic sequencing. Various restriction enzymes are available that digest demethylated sequences, while leaving methylated sequences intact. An advantage of methylation-sensitive restriction enzyme analysis is that it produces DNA fragments that have 5′ and 3′ ends that were demethylated at the time of digestion. As a result it is a quick method of localizing demethylated sequences within a particular restriction sequence within a larger DNA sequence, such as a locus, chromosome, or even a whole genome. Methylation-sensitive restriction enzyme analysis, as well as examples of various methylation-sensitive restriction enzymes, are described in greater detail below.

Methylation-sensitive DNA sequencing, while not as quick a method as restriction enzyme analysis, can provide specific sequence information with regards to any methylation site, regardless of its inclusion within a restriction enzyme site. Maxam and Gilbert chemical cleavage sequencing protocols have been modified and developed to determine methylation status of sequences within a gene, with the absence of a band in all tracks of a sequencing gel indicating the presence of a 5-methylcytosine residue (Church and Gilbert (1984) Proc Natl Acad Sci USA 81:1991-95; Saluz and Jost (1989) Proc Natl Acad Sci USA 86:2602-6; Pfeifer G P, et al. (1989) Science 246:810-13).

Another method of methylation-sensitive DNA sequencing involves exposing genomic DNA to sodium bisulfite (Frommer M, et al. (1992) A genomic sequencing protocol that yields a positive display of 5-methylcytosine residues in individual DNA strands) under conditions where cytosine residues are converted to uracil residues, while 5-methylcytosine residues remain nonreactive. One or both strands of the bisulfite-modified genomic DNA can then be PCR amplified using pairs of strand specific primers. As the bisulfite reaction protocol produces single DNA strands that can no longer achieve 100% complementary basepairing (for example reacting double stranded DNA consisting of 5′-TCTC-3′ base paired to 5′-GAGA-3′ with sodium bisulfite yields single strands of 5′-TUTU-3′ and 5′-GAGA-3′ such that 100% complementary base pairing can no longer be achieved), pairs of PCR primers can be designed such that they anneal in a strand-specific fashion and produce PCR products for each of the single bisulfite-modified DNA strands. The PCR products can then be subject to any combination of assays available to skilled persons including, without limitation, sequencing, cloning, methylation specific PCR, Ms-SNuPe, or microarrays. Bisulfite-modified DNA templates can be conveniently produced using the EZ DNA methylation Kit™ developed by Zymo Research.

The combination of methylation-specific technology and array technology may be particularly useful for high throughput applications. For example, fragments of bisulfite-modified DNA could be analysed using microarrays having probes that were specific for identified hypomethylated sequences. As another example, an array of primers could be developed for analysing each potential demethylation site by Ms-SNuPe assay within a DNA sequence, such as a locus, chromosome, or even a whole genome.

The above techniques can also be used in diagnosis of disease. For example, once one or more than one hypomethylated sequence have been correlated with a disease state, DNA obtained from a subject having the disease can be treated with sodium bisulfite, followed by Ms-SNuPe or methylation-specific PCR using primers that are specific for the correlated hypomethylated sequence(s). As another example, diagnosis of disease can be achieved by digesting DNA, from a diseased sample, with a methylation-sensitive restriction enzyme that yields a different size fragment when digesting DNA from a diseased sample compared to DNA obtained from a normal sample; determination of the disease-specific restriction fragment size can be achieved through any standard method including, Southern analysis.

It will be understood that diagnostic methods of the present invention may be used to identify the presence of a disease in a subject, or may be used to identify a predisposition of a subject to develop a disease. As such the diagnostic methods of the present invention encompass pre-diagnosis of disease.

Accordingly, the present invention is directed to a method of diagnosing an epigenetic abnormality correlated with a disease comprising identifying a hypomethylated sequence within a locus that has an endogenous multi-copy DNA element, wherein the hypomethyated sequence is methylated in a normal sample. The strength of correlation between the presence of a particular hypomethylated sequence and a disease may vary. The strength of correlation can be expressed in terms of percentage of true positives (the number of people who develop a disease divided by the number of people who test positive). Example 2 shows a 100% correlation between Huntingdon's disease and the presence of a locus having a hypomethylated sequence and an Alu sequence (the Alu sequence being located ˜4 Kb downstream of the (CAG)n/(CTG)n repeat region of the HD gene). As such Huntingdon's disease is an example of a particularly successful use of the diagnostic methods of the present invention. Furthermore, the diagnostic methods of the present invention can be successfully used in cases where strength of correlation between disease and hypomethylated sequence is lower than 100%, and could be as low as 50%, 40%, 30% or 20%, or even lower. The strength of correlation that is required for successful use of the diagnostic methods of the invention may depend on several factors that can be ascertained by persons skilled in the art, one of these factors being the strength of correlation provided by diagnostic methods that are available in the marketplace. For example, in a disease where no diagnostic method is currently available the diagnostic methods of the present invention may be useful even if providing a strength of correlation that is lower than 20%. Persons skilled in the art will recognize, that strength of correlation may include other factors in addition to the percentage of true positives, for example, a percentage of false positives (the number of people who do not develop a disease divided by the number of people who test positive). Again, as was the case for the desired percentage of true positives, the percentage of false positives that can be tolerated may depend on the number of false positives being generated by commercially available diagnostic methods.

Identification of hypomethylated sequences and endogenous multi-copy DNA elements can be accomplished using any suitable technique, or any other technique that is convenient to the skilled technician. In order to illustrate the variability that can be incorporated in the present method for identifying a locus that has a hypomethylated sequence and a retroelement, for example, an Alu retroelement, the following non-limiting protocols are provided:

Protocol (A)

a) digest genomic DNA with a methylation-sensitive restriction enzyme (which digests hypomethylated sequences) to produce a pool of restricted DNA fragments,

b) fractionate the pool of restricted DNA fragments to obtain DNA fragments of a desired size,

c) amplify at least a segment of the DNA fragments of a desired size with primers that anneal to an Alu sequence to produce a PCR product having at least a portion of the Alu sequence,

d) determine the sequence the PCR product, and

e) compare said sequence against a genomic database to assign a locus for the PCR product having the at least a portion of the Alu sequence.

Protocol (B)

a) determine locations of Alu sequences in silico within a genomic database to obtain dataset of loci having Alu sequences,

b) modify genomic DNA from test and control samples by reacting with sodium bisulfite whereby cytosine is converted to uracil while 5-methylcytosine is unreacted,

c) amplify one or both strands of the converted DNA using pairs of strand-specific primers (primers are chosen such that they flank the Alu sequence at an appropriate distance, for example, 10 kilobases) to produce one (if only one strand amplified) or two (if both strands amplified) PCR products per loci under investigation,

d) (i) identify hypomethylated sequences by sequencing PCR products and identifying a C to T conversion in PCR product sequences derived from test samples compared to a lack of a C to T conversion in a corresponding nucleotide position in PCR product sequences derived from control samples; or

(ii) identify hypomethylated sequence by comparing test and control PCR products treated with restriction enzyme(s) that are appropriately chosen to distinguish between a methylated and bisulfite unreacted CG or CNG sequence versus a demethylated and bisulfite converted TG or TNG sequence (to obtain predicted methylated and demethylated restriction maps any standard software can be used to convert all CG to XG then convert all C to T then convert all X to C and then produce a software predicted restriction map to obtain a methylated map, while conversion of all C to T followed by producing a software predicted restriction map provides a demethylated map), or

(iii) identify hypomethylated sequence by comparing test and control PCR products in Ms-SNuPe assay (Gonzalgo and Jones (1997) Rapid quantitation of methylation differences at specific sites using methylation-sensitive single nucleotide primer extension (Ms-SNuPe) Nucleic Acids Research 25:2529-31) for each potential demethylatation site (an advantage of this technique is that multiple methylation sites can be analysed in each by using a multiplex primer strategy with primers being designed to terminate immediately upstream of each methylation site in accordance with analysis of sequences flanking the identified Alu sequence), or

(iv) identify hypomethylated sequence by comparing the test and control PCR products in methylation-specific PCR assays where primers are designed for differential primer annealing to an in silico predicted methylation site on the basis of bisulfite-induced C to T conversions;

Protocol (C)

a) determine locations of Alu sequences in silico within a genomic database to obtain dataset of loci having Alu sequences,

b) modify genomic DNA from test and control samples by reacting with sodium bisulfite whereby cytosine is converted to uracil while 5-methylcytosine is unreacted, and

c) identify hypomethylated sequence by comparing the test and control bisulfite-modified genomic DNA samples in methylation-specific PCR assays where primers are designed for differential primer annealing to an in silico predicted methylation site on the basis of bisulfite-induced C to T conversions;

Protocol (D)

a) identify locations of potential demethylation sites in silico within a genomic database to obtain dataset of loci having potential demethylation sites, modify genomic DNA from test and control samples by reacting with sodium bisulfite whereby cytosine is converted to uracil while 5-methylcytosine is unreacted,

b) amplify bisulfite-converted DNA using strand-specific primers (primers are chosen such that they flank the potential demethylation site(s)) to produce PCR products,

c) identify hypomethylated sequence by comparing test and control PCR products in Ms-SNuPE assay for each potential demethylatation site to obtain an array of PCR products and loci having hypomethylated sequence(s),

d) (i) determine locations of Alu sequences in silico within dataset of loci having hypomethylated sequence(s), or

(ii) identify Alu sequences within the array of PCR products by any standard technique, for example, without limitation, Southern assay or PCR or DNA sequencing;

or,

Protocol (E)

a) identify locations of potential demethylation sites in silico within a genomic database to obtain dataset of loci having potential demethylation sites, modify genomic DNA from test and control samples by reacting with sodium bisulfite whereby cytosine is converted to uracil while 5-methylcytosine is unreacted,

b) amplify bisulfite-converted DNA using strand-specific primers (primers are chosen such that they flank the potential demethylation site(s)) to produce PCR products,

c) identify hypomethylated sequence by sequencing test and control PCR products and identifying a C to T conversion in PCR product sequences derived from test samples compared to a lack of a C to T conversion in a corresponding nucleotide position in PCR product sequences derived from control samples,

d) (i) determine locations of Alu sequences in silico within dataset of loci having hypomethylated sequence(s),

(ii) identify Alu sequences within the array of PCR products by any standard technique, for example, without limitation, Southern assay or PCR or DNA sequencing;

Any of the above protocols can be used to identify loci having a hypomethylated sequence and a multi-copy DNA element within a test sample compared to a control sample. Usually the test sample will be the genome of diseased tissue, while the control sample can be a corresponding tissue in a person not suffering from the disease. However, persons skilled in the art will recognize other relevant test/control comparisons such as the control sample being any normal tissue from within a diseased animals own body (for example, cancerous liver tissue samples could be compared to non-cancerous liver tissue samples with both samples obtained from within the same subject). The methods of the present invention can be applied to any disease that occurs as a result of hypomethylation within a locus having an endogenous multi-copy DNA element, including both Mendelian and non-Mendelian disease. Illustrative examples of diseases include, without limitation, cystic fibrosis, Duchennes muscular dystrophy, Huntington's disease, fragile X syndrome, schizophrenia, bipolar disorder, cancers and diabetes.

DNA analysed in accordance with methods of the present invention may be extracted from any sample that may have epigenetic abnormalities associated with a disease, for example, but not limited to cells of the following tissues: Epithelial Tissues, Exocrine Glands, Endocrine Glands, Connective Tissues, Adipose Tissue, Cartilage, Bone, Blood, Muscle Tissues comprising Smooth, Skeletal or Cardiac Muscle Tissue, or Nervous Tissue comprising Brain Tissue. DNA can be extracted using standard techniques, known in the art, for isolating DNA from various samples such as cells, tissues, or organs, or other suitable specimens. Standard techniques for isolating DNA have are disclosed in reference textbooks or manuals such as Sambrook, Fritsch, and Maniatis, Molecular Cloning: A Laboratory Manual (1989), Cold Spring Harbor.

The above-described non-limiting illustrative protocols specify the identification of Alu sequences. However, the methods of the invention are equally applicable to other endogenous multi-copy DNA elements, for example, but not limited to, an L1 seqeunce, a SINE sequence, a LINE sequence, or an endogenous retroviral sequence (ERV).

A method of the present invention is directed to identifying a locus that has an increased probability of causing a diseased state comprising identifying a locus, within a genome obtained from a diseased sample, that has a hypomethylated sequence and an endogenous multi-copy DNA element, wherein the hypomethylated sequence is methylated in a normal sample. An advantage of this method is that it provides a short cut for identification of causal factors of a disease, and further provides a short cut to identification of drug targets to treat disease. By concentrating on loci that have both a disease-specific hypomethylated sequence and an endogenous multi-copy DNA vast stretches of genomic DNA can be eliminated from analysis, and analysis can be focused on DNA coding sequences that are proximal to, or comprise, the endogenous multi-copy DNA element and disease-specific hypomethylated sequence. For example, this assay may select from about 1 to about 10 DNA coding sequences from the disease-specific hypomethylated locus. By “DNA coding sequence” it is meant an open reading frame as commonly understood in the art

Techniques for analysing expression profiles of surrounding genes including, but not limited to, Northern, ELISA, reporter construct assays, microarray assay of RNA levels, dot blots, quantitative PCR, are well known to persons skilled in the art, and are not critical to the present invention. Any number of standard and available techniques may be used to determine which of the genes proximal to a locus, identified in accordance with the present invention, are aberrantly regulated in a diseased state. The present invention provides for a quick way to focus available analytical resources on a set of about 1 to about 10 DNA coding sequences that are found to be surrounding or within a locus that has a disease-specific hypomethylated sequence and an endogenous multi-copy DNA element. Usually, the dys-regulated gene which causes the diseased state will be found within the locus, or within a nucleotide sequence defined by the distance of about 1 to about 10 DNA coding sequences, and will be typically located within 1 to about 200 kilobases of the identified disease-specific hypomethylated locus. However, as seen in Table 3 this separation may be less than 200 Kb and may vary, for example, without limitation, from about 100 Kb, to about 50 Kb, to about 5 Kb, to almost overlapping with the identified disease-specific hypomethylated locus.

By “dys-regulated gene” or “aberrantly regulated gene” it is meant a nucleotide sequence that is differentially regulated between a diseased and non-diseased sample.

The number of DNA coding sequences of less than about 10 compares favourably to a relatively larger range of 5 to 300 genes often contained within chromosomal regions identified by traditional genetic linkage studies. In a further aspect, a DNA coding sequence having an epigenetically altered expression pattern that contributes to a disease in an organism can be identified by comparing expression patterns of the DNA coding sequence located proximal to the disease-specific hypomethylated locus within a test sample that exhibits characteristics of said disease with expression patterns of a corresponding DNA coding sequence within a control sample to identify the DNA coding sequence having an epigenetically altered expression pattern. The DNA coding sequence may encode an RNA that remains non-translated, or may encode an RNA that is translated, at least partially, into a polypeptide.

A method of the present invention is directed to detection of epigenetic abnormalities associated with a non-Mendelian disease and comprises extraction of genomic DNA from a non-Mendelian disease sample, such as diseased tissue or diseased population of cells; hydrolysis of this DNA with methylation-sensitive restriction enzymes, and subsequent fractionation of DNA fragments and purification of DNA fragments of a desired size, for example, but not limited to, shorter than 10 kB. These purified DNA fragments are further subjected to PCR amplification using primers that hybridize to endogenous multi-copy DNA elements including, but not limited to, ALU or L1 elements. After that, PCR products of such elements are cloned and sequenced using standard molecular biology techniques known to the skilled artisan and the resultant sequences are mapped on the genome using any commercially or publicly available human genome database. These cloned multi-copy elements indicate a loci of putative epigenetic abnormality or epigenetic dys-regulation and indicates genes that predispose a patient to a complex, non-Mendelian, multi-factorial disease, such as, but not limited to, cancers, diabetes, schizophrenia, or bipolar disorder. Persons skilled in the art will recognize that this method can be used in regards to any disease, both non-Mendelian and Mendelian.

By the term “non-Mendelian disease” is meant any disease which etiologically requires more than a single genetic abnormality. As such a non-Mendelian disease requires more than one factor, or in other words, is multi-factorial, and may comprise epigenetic alterations or abnormalities.

Epigenetics relates to higher order gene control mechanisms in eukaryotes that activate or repress parts of the genome via changes in chromatin structure. These higher order gene control mechanisms form an important molecular basis of cell differentiation. Any changes in an organism brought about by alterations in the action of genes, where the changes do not require occurrence of any mutations, are called epigenetic changes. An epigenetic abnormality occurs when an epigenetic change contributes or predisposes normal cells into becoming diseased cells. DNA methylation is an example of an epigenetic mechanism. The term DNA methylation refers to the addition of a methyl group to the cyclic carbon 5 of a cytosine nucleotide. A family of conserved DNA methyltransferases catalyzes this reaction. Normally, DNA methylation can be used, for example, but is not limited to, to methylate the transcription unit of a gene so that the gene is turned off or silenced, and a corresponding protein product is not produced in a particular cell. For instance, one of the two X chromosomes in female mammals is inactivated or silenced by methylation.

DNA is extracted from a non-Mendelian disease sample using standard techniques, known in the art, for isolating DNA from various samples such as cells, tissues, or organs, or other suitable specimens. Standard techniques for isolating DNA have are disclosed in reference textbooks or manuals such as Sambrook, Fritsch, and Maniatis, Molecular Cloning: A Laboratory Manual (1989), Cold Spring Harbor.

DNA may be extracted from any sample that may have epigenetic abnormalities associated with a non-Mendelian disease or any sample that exhibits characteristics of a non-Mendelian disease, for example, but not limited to cells of the following tissues: Epithelial Tissues, Exocrine Glands, Endocrine Glands, Connective Tissues, Adipose Tissue, Cartilage, Bone, Blood, Muscle Tissues comprising Smooth, Skeletal or Cardiac Muscle Tissue, or Nervous Tissue comprising Brain Tissue.

Any methylation-sensitive restriction enzyme may be used for the purposes of this invention. The terms “restriction endonucleases” and “restriction enzymes” refer to bacterial enzymes, each of which cut double-stranded DNA at or near a specific nucleotide sequence. The process of cutting or cleaving the DNA is referred to as restriction digestion. The products of a restriction digestion are referred to as restriction products. A restriction enzyme used in the present invention may yield restriction products having blunt-ends or overhanging “sticky” ends. Specifically, a restriction enzyme can symmetrically cut both strands of a double stranded DNA fragment to produce a blunt-ended fragment, or a restriction enzyme may assymetrically cleave the two strands of a DNA fragment to produce a DNA fragment that has a single stranded overhang. In general, a methylation-sensitive restriction enzyme used in the present invention will recognize and cleave a non-methylated sequence, while it will not cleave a corresponding methylated sequence. Methylation of plant and mammalian DNA occurs at CG or CNG sequences. This methylation may interfere with the cleavage by some restriction endonucleases. Endonucleases that are sensitive and not sensitive to m⁵CG or m⁵CNG methylation, as well as isoschizomers of methylation-sensitive restriction endonucleases that recognize identical sequences but differ in their sensitivity to methylation, can be extremely useful for studying the level and distribution of methylation in eukaryotic DNA. Examples of methylation-sensitive restriction enzymes, and corresponding restriction site sequences, that can be used according to the present invention include, but are not limited to: AatII (GACGTC); Bsh1236I (CGCG); Bsh1285I (CGRYCG); BshTI (ACCGGT); Bsp68I (TCGCGA); Bsp119I (TTCGAA); Bsp143I (RGCGCY); Bsu15I (ATCGAT); Cfr10I (RCCGGY); Cfr42II (CCGCGG); CpoI (CGGWCCG); Eco47III (AGCGCT); Eco52I (CGGCCG); Eco72I (CACGTG); Eco105I (TACGTA); EheI (GGCGCC); Esp3I (CGTCTC); FspAI (RTGCGCAY); Hin1I (GRCGYC); Hin6I (GCGC); HpaII (CCGG); Kpn2I (TCCGGA); MluI (ACGCGT); NotI (GCGGCCGC); NsbI (TGCGCA); PauI (GCGCGC); PdiI (GCCGGC); Pfl23II (CGTACG); Psp1406I (AACGOT); PvuI (CGATCG); SalI (GTCGAC); SmaI (CCCGGG); SmuI (CCCGC); TaiI (ACQT); or TauI (GCSGC).

Size fractionation and purification of restricted DNA fragments can be performed by any method known in the art, for example, but not limited to, separation of DNA fragments of a desired size such as fragments of less than 10 kB by centrifugation of a DNA fragment pool through a membrane or other suitable matrix having size exclusion or inclusion properties. Alternatively, a pool of restricted DNA fragments may be separated using agarose of polyacrylamide gel electrophoresis and DNA fragments of a desired-size may be purified using any suitable gel-extraction composition such as glass milk or Quaternary ammonium ions. The desired size limit of the fractionated and isolated DNA fragments depends on the size of the endogenous DNA element that serves as a template for PCR amplification. As such the “DNA fragments of a desired size” can be any size as long as they are larger than, and can therefore comprise the endogenous DNA element.

As used, the terms “amplification,” “amplify,” or “amplifying,” are defined as the production of additional copies of a nucleic acid sequence and is generally carried out using polymerase chain reaction (PCR) or other technologies well known in the art (e.g., Dieffenbach and Dveksler, PCR Primer, a Laboratory Manual, Cold Spring Harbor Press, Plainview N.Y. [1995]). Nucleic acid amplification techniques allow for increasing the concentration of a target or template sequence, or a portion or segment thereof from a mixture of genomic DNA without cloning or purification. A review of current nucleic acid amplification technology can be found in Kwoh et al., 8 Am. Biotechnol. Lab. 14 (1990). In vitro nucleic acid amplification techniques include polymerase chain reaction (PCR), transcription-based amplification system (TAS), self-sustained sequence replication system (3SR), ligation amplification reaction (LAR), ligase-based amplification system (LAS), Q.beta. RNA replication system and run-off transcription. All present and future nucleic acid amplification technology can be incorporated into the present invention.

PCR is a preferred method for DNA amplification. PCR synthesis of DNA fragments occurs by repeated cycles of heat denaturation of DNA fragments, primer annealing onto endogenous sequence elements or exogenous adaptor ends of a DNA fragment or other suitable DNA template, and primer extension. These cycles can be performed manually or, preferably, automatically. Thermal cyclers such as the Perkin-Elmer Cetus cycler are specifically designed for automating the PCR process, and are preferred. The number of cycles per round of synthesis can be varied from 2 to more than 50, and is readily determined by considering the source and amount of the nucleic acid template, the desired yield and the procedure for detection of the synthesized DNA fragment.

PCR techniques and many variations of PCR are known. Basic PCR techniques are described by Saiki et al. (1988 Science 239:487-49,1) and by K. B. Mullis in U.S. Pat. Nos. 4,683,195, 4,683,202 and 4,800,159, which are incorporated herein by reference.

The conditions generally required for PCR include temperature, salt, cation, pH and related conditions needed for efficient amplification of at least a segment or portion of a DNA fragment template. PCR conditions include repeated cycles of heat denaturation, and incubation at a temperature permitting primer hybridization to an endogenous sequence elements or exogenously ligated adaptors, and copying of the DNA fragment by the amplification enzyme. Heat stable amplification enzymes like the pwo, Thermus aquaticus or Thermococcus litoralis DNA polymerases are commercially available which eliminate the need to add enzyme after each denaturation cycle. The salt, cation, pH and related factors needed for enzymatic amplification activity are available from commercial manufacturers of amplification enzymes.

As provided herein an amplification enzyme is any enzyme which can be used for in vitro nucleic acid amplification, e.g. by the above-described procedures. Amplification enzymes may be thermostable or thermolabile. Such amplification enzymes include pwo, Escherichia coli DNA polymerase I, Klenow fragment of E. coli DNA polymerase I, T4 DNA polymerase, T7 DNA polymerase, Thermus aquaticus (Taq) DNA polymerase, Thermococcus litoralis DNA polymerase, SP6 RNA polymerase, T7 RNA polymerase, T3 RNA polymerase, T4 polynucleotide kinase, Avian Myeloblastosis Virus reverse transcriptase, Moloney Murine Leukemia Virus reverse transcriptase, T4 DNA ligase, E. coli DNA ligase, Vent polymerases, or Q.beta. replicase. Preferred amplification enzymes are the pwo and Taq polymerases. The pwo enzyme is especially preferred because of its fidelity in replicating DNA.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

By the term “primer” is meant an oligonucleotide, whether occurring naturally as in a purified restriction digest or produced synthetically, capable of acting as a point of initiation of synthesis when placed under suitable conditions in which synthesis of a primer extension product that is complementary to a nucleic acid strand is induced. Such suitable conditions comprise nucleotides and an amplification enzyme such as DNA polymerase and a suitable temperature, salt concentration, and pH). The primer is preferably single stranded for maximum efficiency in amplification, but may alternatively be double stranded. If double stranded, the primer is first treated to separate its strands before being used to prepare extension products. The primer must be sufficiently long to prime the synthesis of extension products in the presence of the inducing agent. The exact lengths of the primers will depend on many factors, including temperature, salt concentration, pH, source of primer and the use of the method. The primers of the present invention can hybridize or anneal to a sequence element that is endogenous to a DNA fragment template or the primers can anneal to exogenous adaptor sequence elements that have been ligated to the ends of a DNA fragment template. Preferably, the primers anneal to an endogenous multi-copy DNA sequence element, for example, long or short interspersed nucleotide elements (LINEs or SINEs).

Endogenous multi-copy DNA elements are repetitive DNA sequences that together are estimated to comprise 30% of total genomic sequences. Present at between 10-10⁵ copies per genome these multi-copy elements can be found throughout the euchromatin and have been categorized as:

a) microsatellites/minisatellites (VNTR, DNA 'fingerprints)

b) dispersed-repetitive DNA, mainly transposable elements (LINES (for example, L1)/SINES(foe example, Alu))

Endogenous multi-copy DNA elements can also include ‘redundant’ genes for histones, endogenous retroviral sequences (ERV), and ribosomal RNA and proteins, (gene-products present in cell in large numbers).

Many multi-copy DNA elements may be involved in regulation of gene expression as they have been shown to be interspersed within single-copy sequences and have been shown to be located proximal to structural genes.

Long and short interspersed nucleotide elements (LINEs and SINEs), are represented in humans mainly by L1 (Furano A V. The biological properties and evolutionary dynamics of mammalian LINE-1 retrotransposons. Prog Nucleic Acid Res Mol Biol. 2000;64:255-94) and Alu elements (Watson et al., Molecular Biology of the Gene, fourth edition (1987) pp. 669-670), respectively. Both types of elements are considered to be retrotransposable (ie. can replicate via an RNA copy reinserted as DNA by reverse transcription) and they have significant roles in genomic function. The inserted elements can be full length or truncated, or may be rearranged relative to full-length elements.

The most common and best characterised LINE is L1, having the following properties

Repeated approximately 50000 times in the human genome (0.5% of total)

Only about 3000 of these are full length; the remainder are truncated, mostly at the 5′ end.

Full length element is about 6 kb in size and contains two open reading frames, one of which encodes a reverse transcriptase.

AT-rich region is located near the 3′ end of the element,

Element is flanked by two short direct repeats.

The main type of SINE is the Alu family, characterized as follows:

usually contain a target for the restriction enzyme Alu I;

5×10⁵-10⁶ copies in the haploid genome, with an average of one repeat every 4 to 5 kb (1-10 % total);

Often present in the transcription unit of a gene, within introns and occasionally in non-translated regions of the mRNA;

Generally contain 300 bp consensus sequence which consist of two tandem repeats of a 130 bp sequence, one of which has a 32 bp deletion, as such Alu family members are recognizably related in sequence, but not precisely conserved;

Elements are flanked by direct repeats;

Each repeat unit has an AT-rich region that suggests a poly A tail;

5′ end resembles a pol III promoter region.

LINEs and SINEs both have a poly(A) tail which may act as a template for reverse transcription from nicks made at the site of insertion in the host DNA by a LINE-encoded endonuclease.

Primers of the present invention may be designed according to any L1 or Alu sequence. For example, various analyses (Claverie, J. M. and Makalowski, W. Alu alert, Nature 371, 752 (1994)) indicate that Alu repeats fall into 8 subfamilies, and therefore, 8 ALU consensus sequences have been constituted and added to GenBank as accession numbers U14567, U14568, U14569, U14570, U14571, U14572, U14573 and U14574. A primer of the present invention may be designed in accordance with any of these consensus sequences. For example, the deposited consensus sequence of a subfamily of Alu repeats designated U14570 is as follows:

(SEQ ID NO:1)

GGCCGGGCGCGGTGGCTCACGCCTGTAATCCCAGCACTTTGGGAGGCCGA
GGCGGGTGGATCATGAGGTCAGGAGATCGAGACCATCCTGGCTAACAAGG
TGAAACCCCGTCTCTACTAAAAATACAAAAAATTAGCCGGGCGCGGTG

Products of amplification reactions can be subjected to sequence determinations. Amplification products, preferably PCR products, can optionally be cloned into a vector before sequencing. When not cloning a PCR product, an adaptor DNA elements can be ligated to the ends of PCR products, and the PCR products can be sequenced using a primer that anneals to the adaptor element. Cloning, ligation, and sequencing can be performed using standard techniques, such as protocols described in textbooks or manuals such as Sambrook, Fritsch and Maniatis, Molecular Cloning: A Laboratory Manual, 1989. Also, commercially available kits may be utilized. Another alternative for sequence determination are automated DNA sequencing systems and methods.

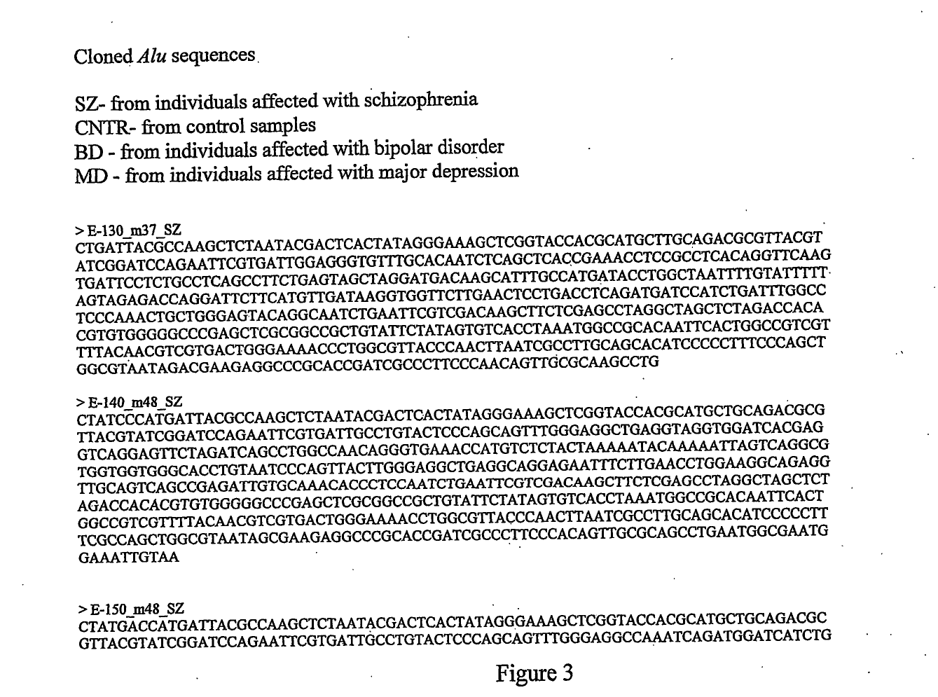

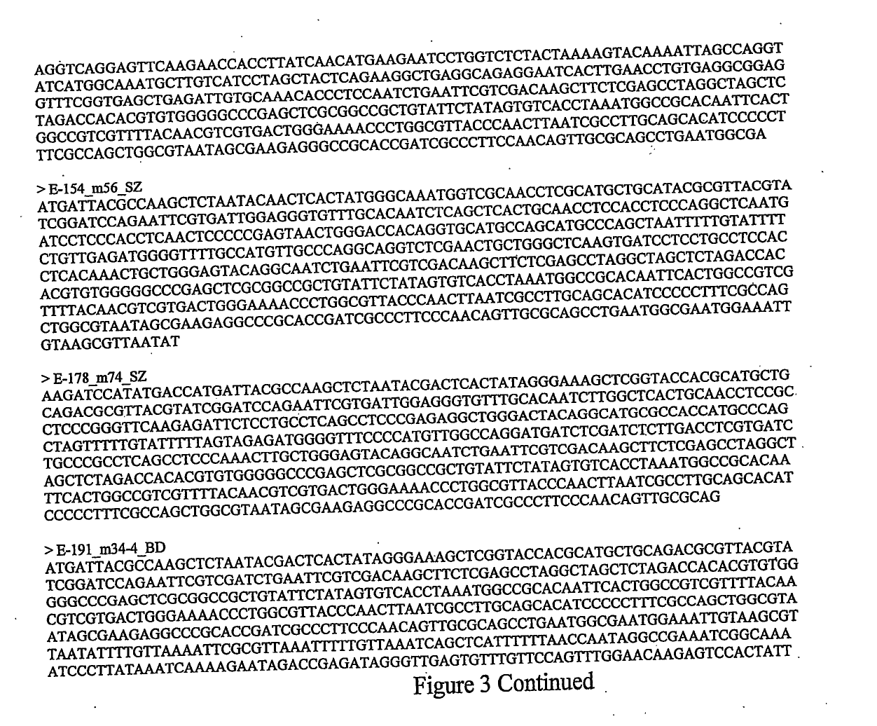

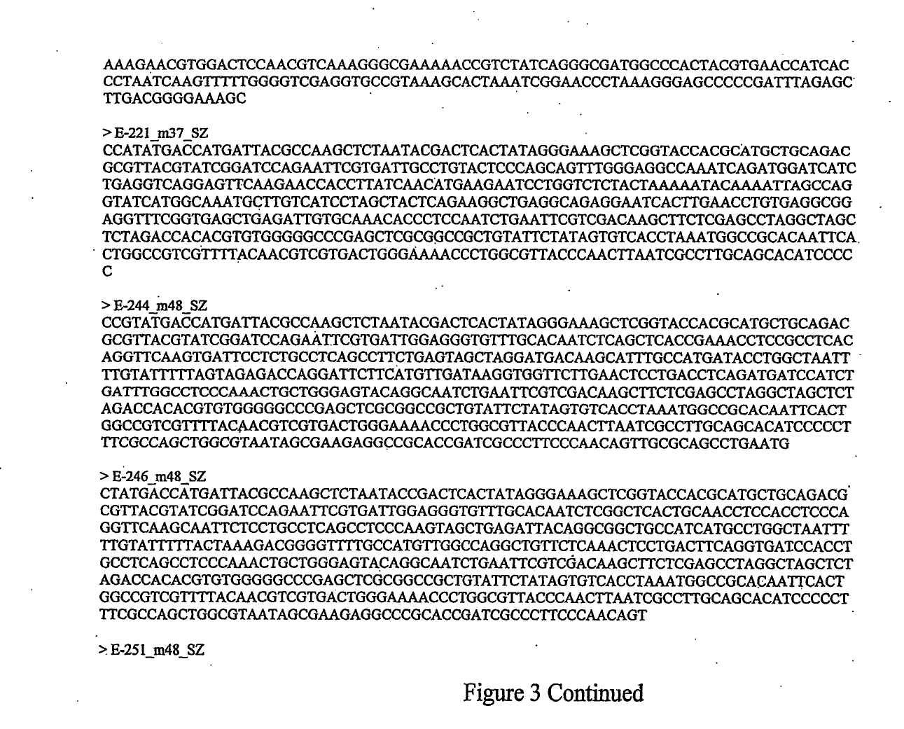

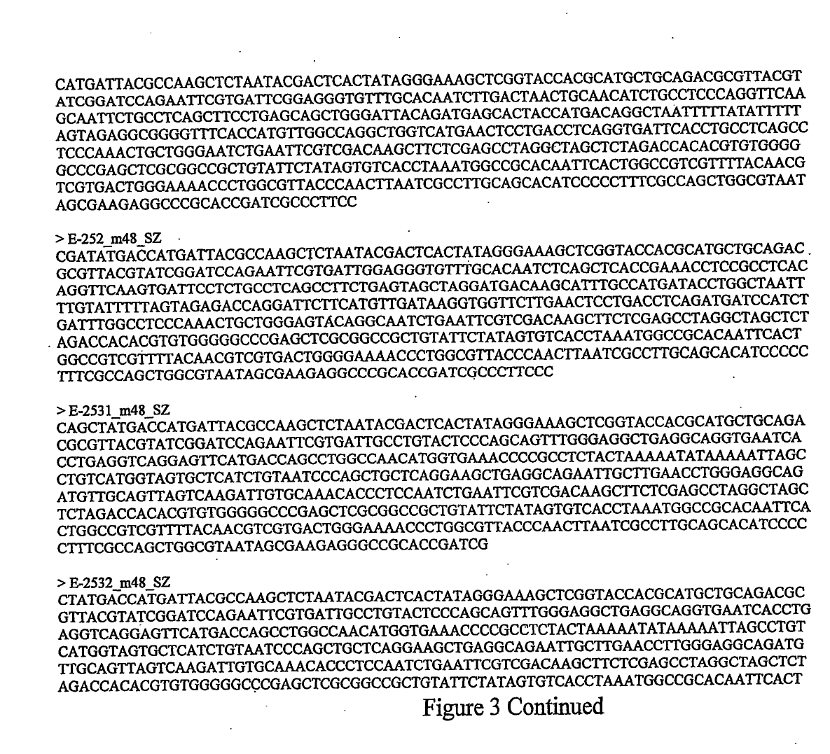

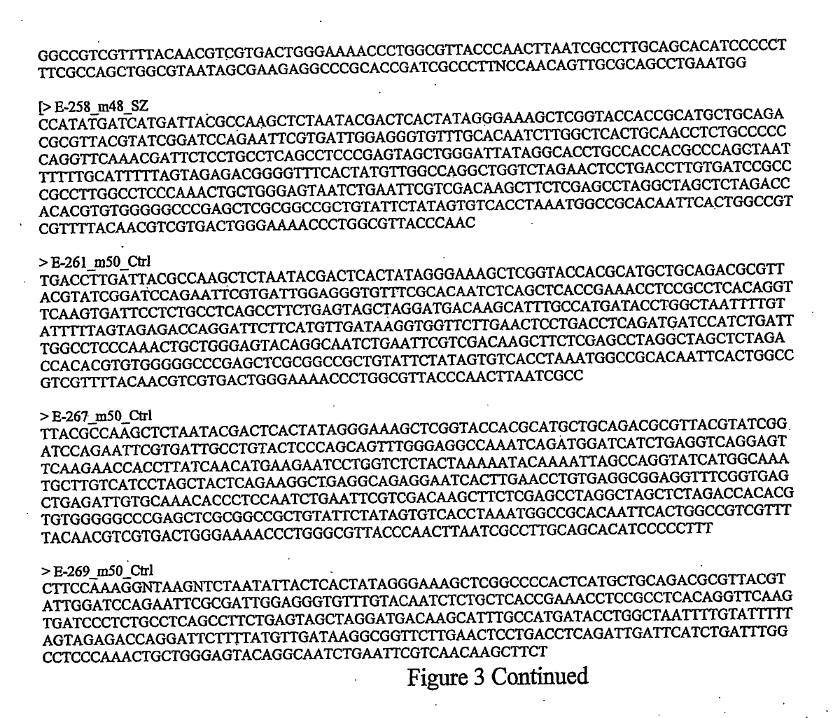

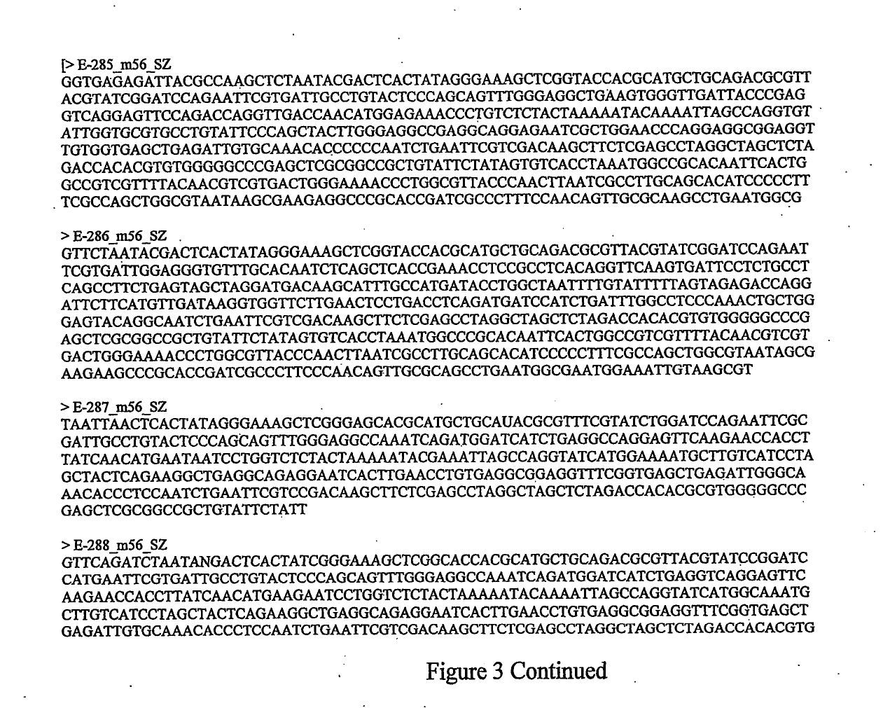

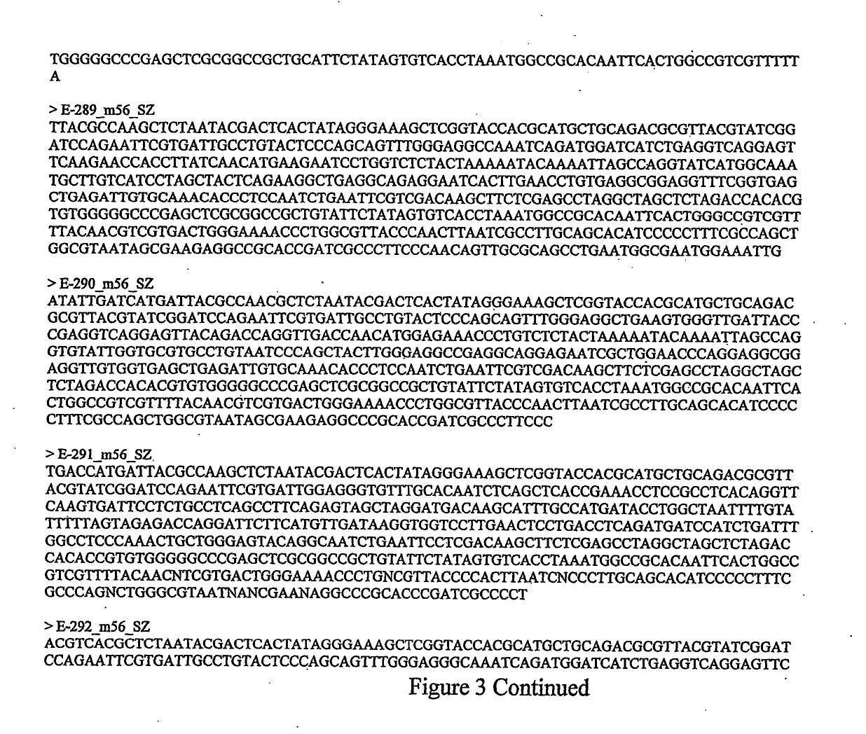

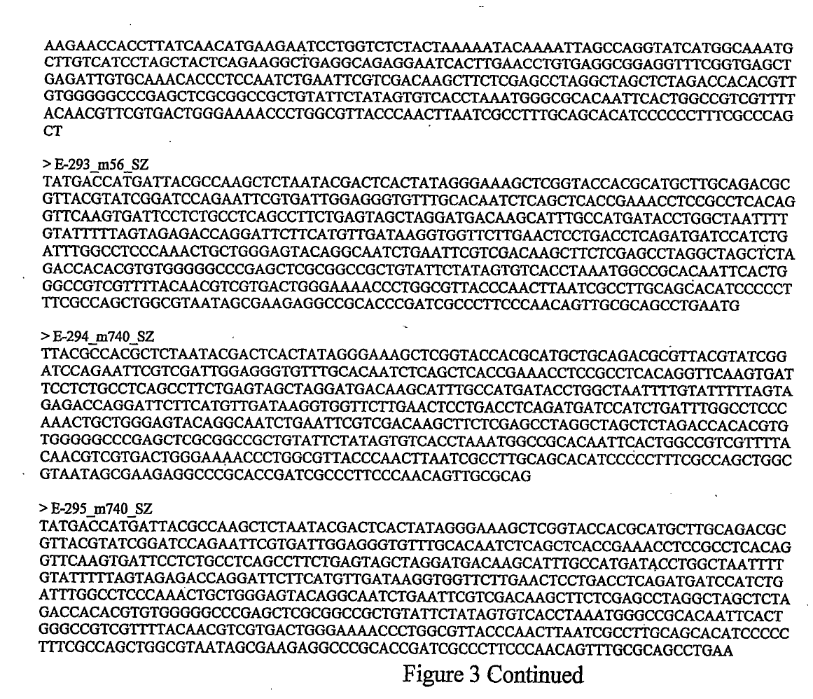

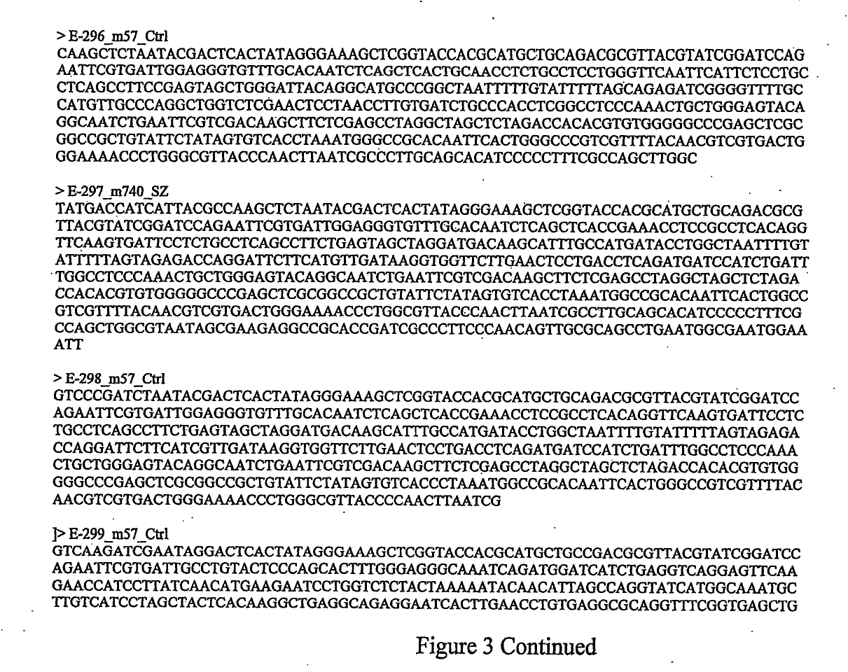

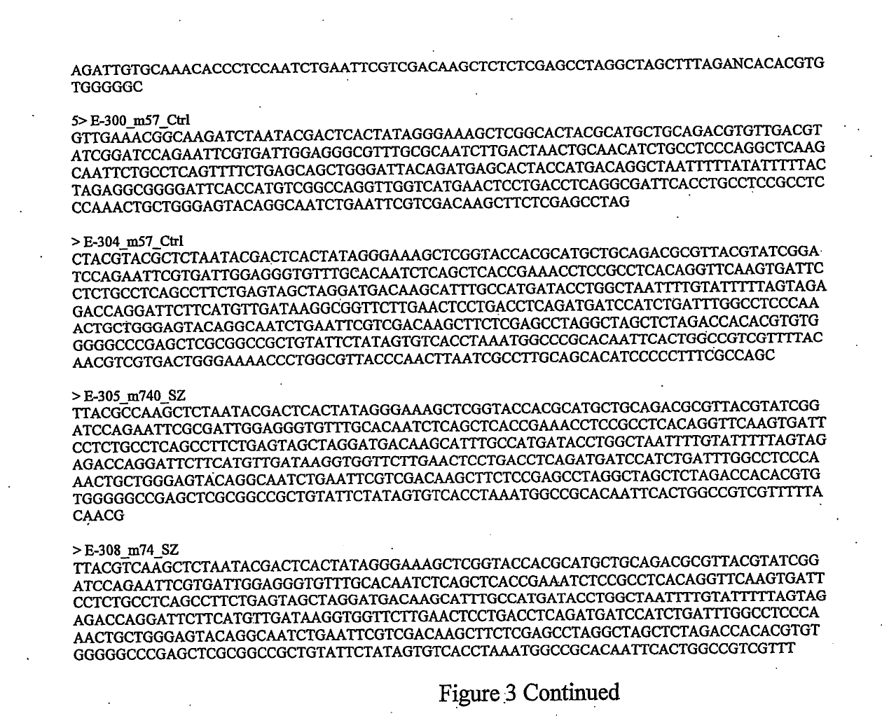

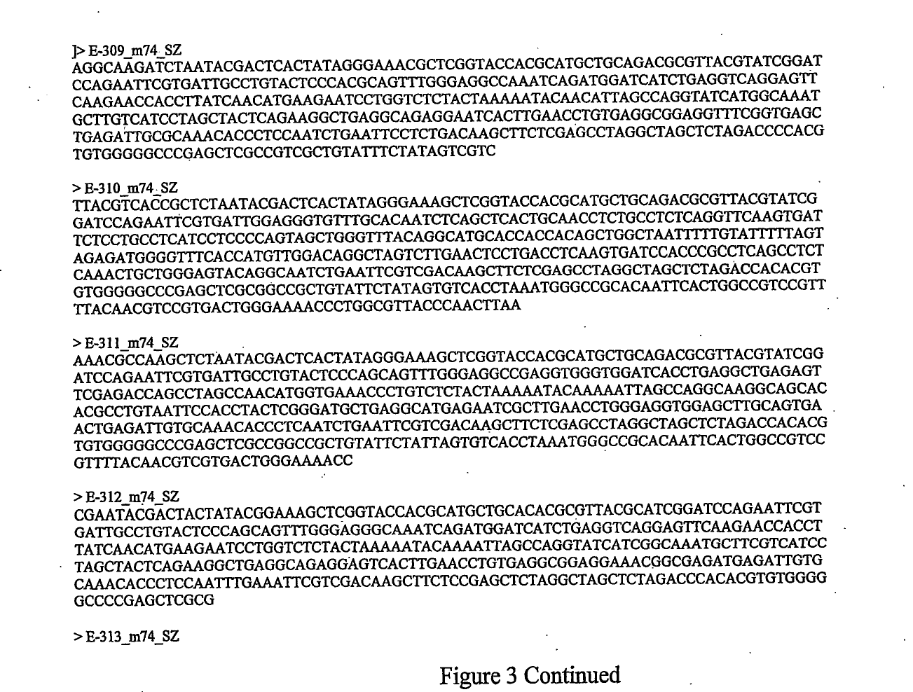

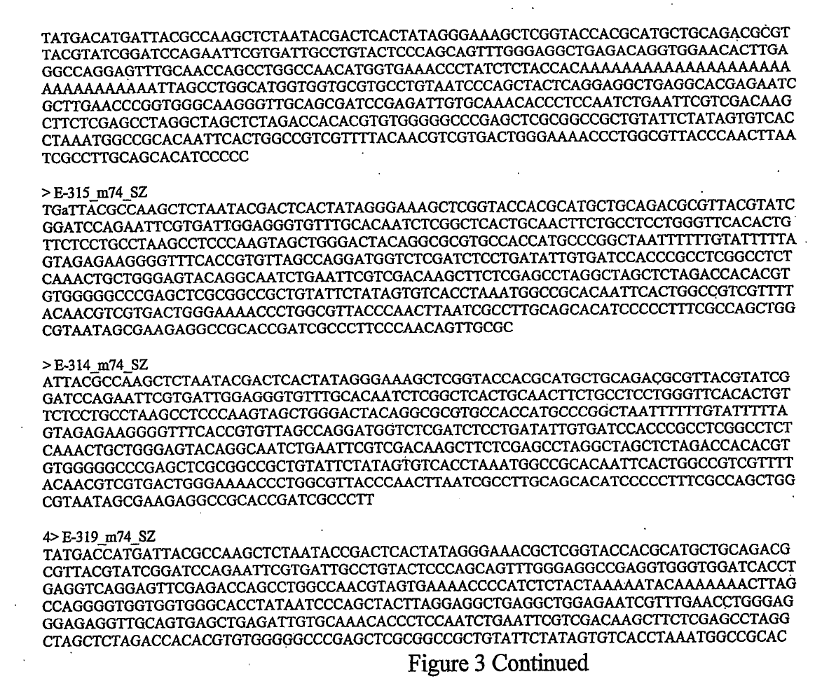

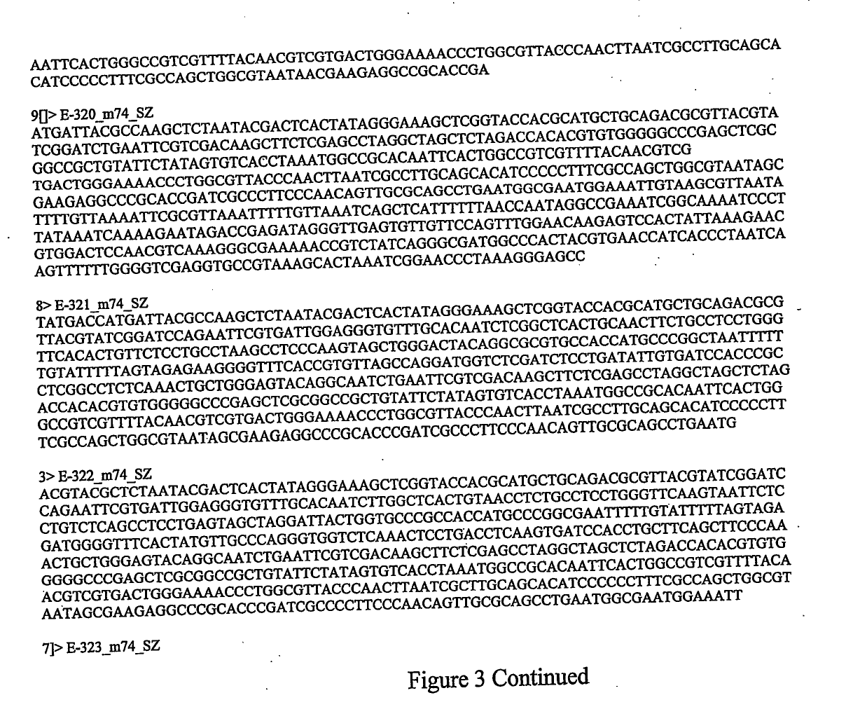

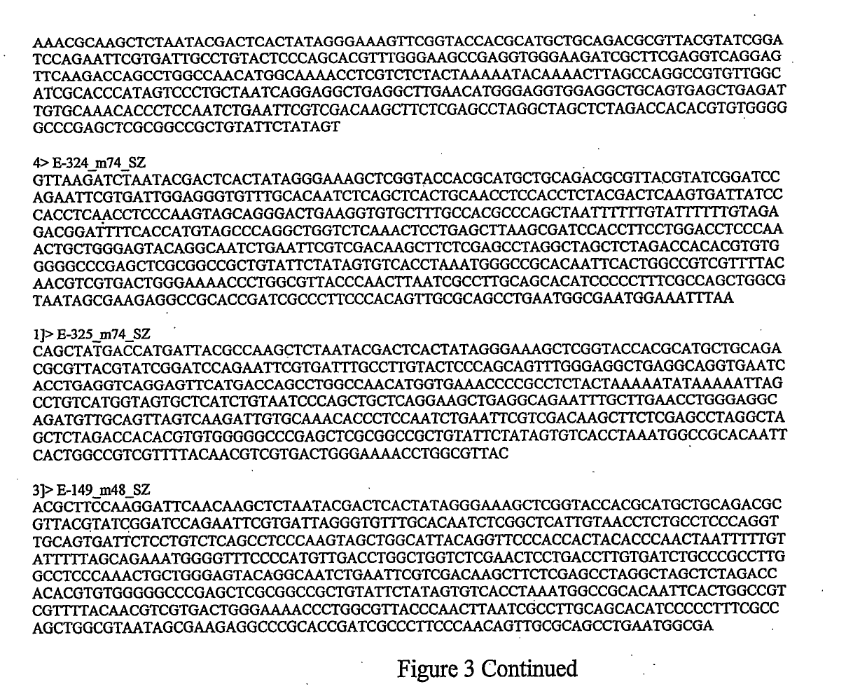

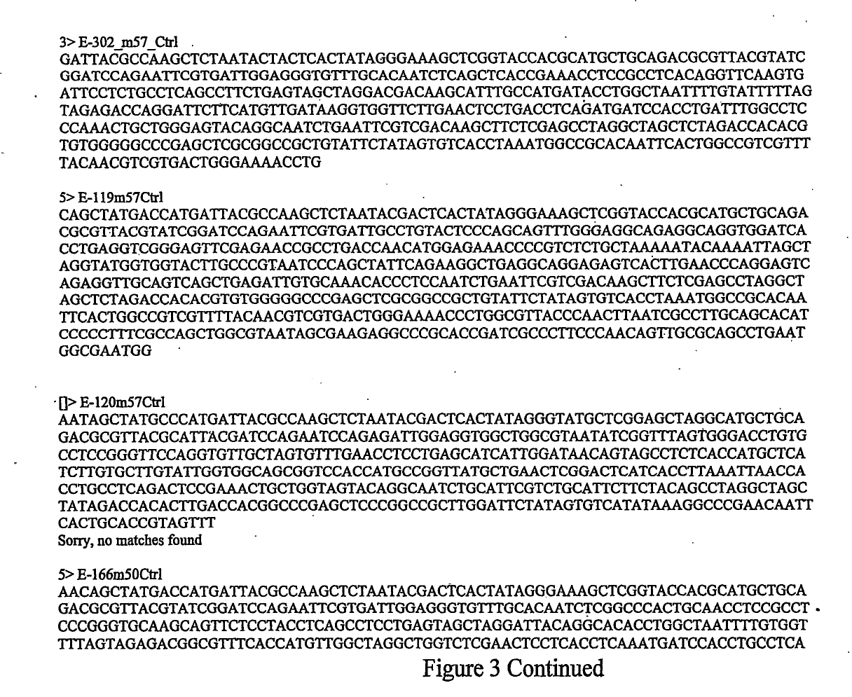

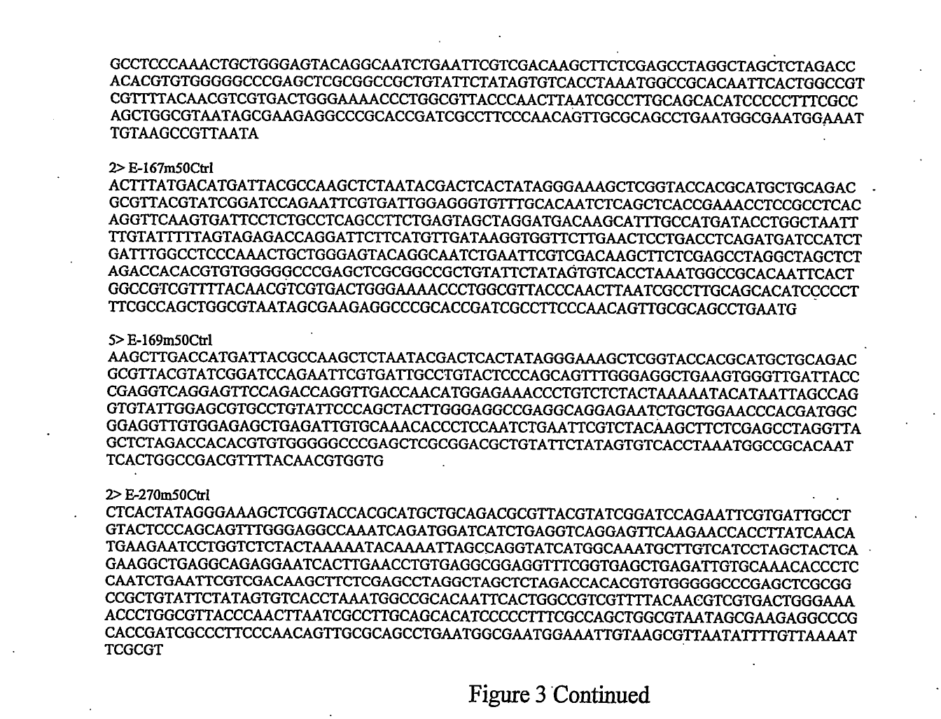

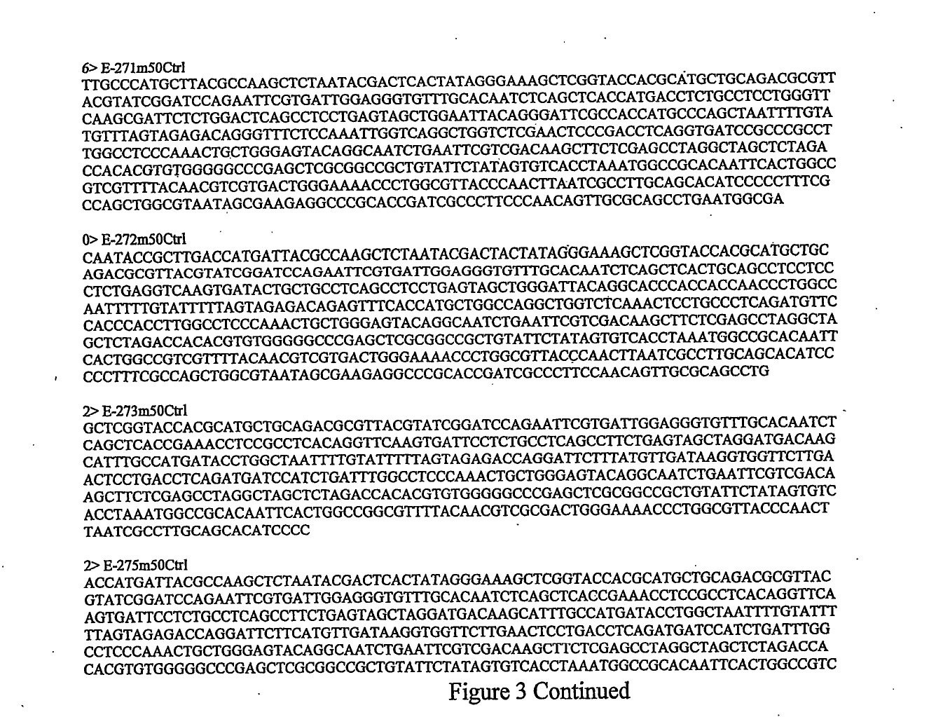

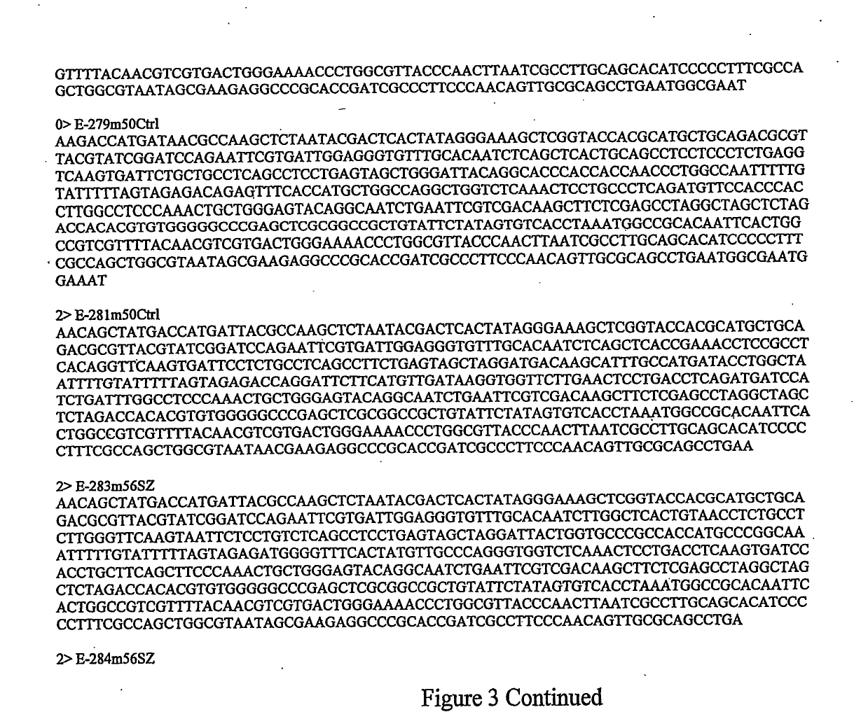

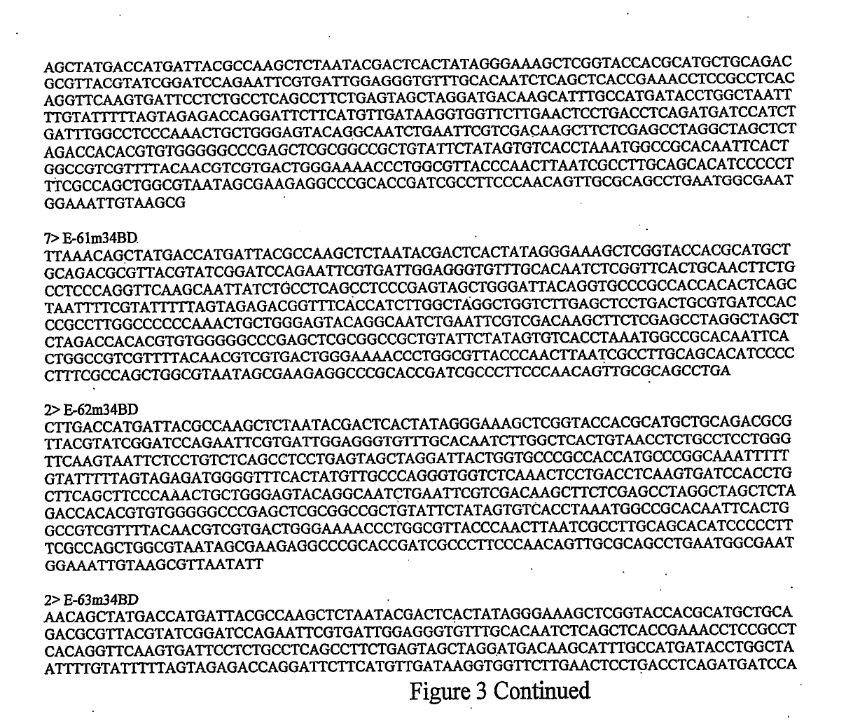

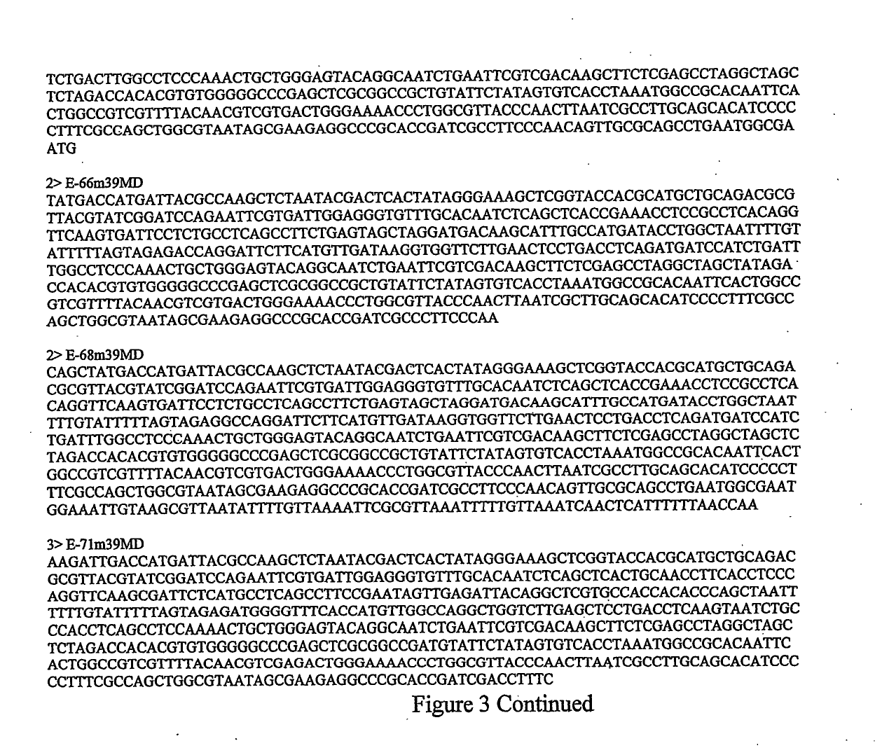

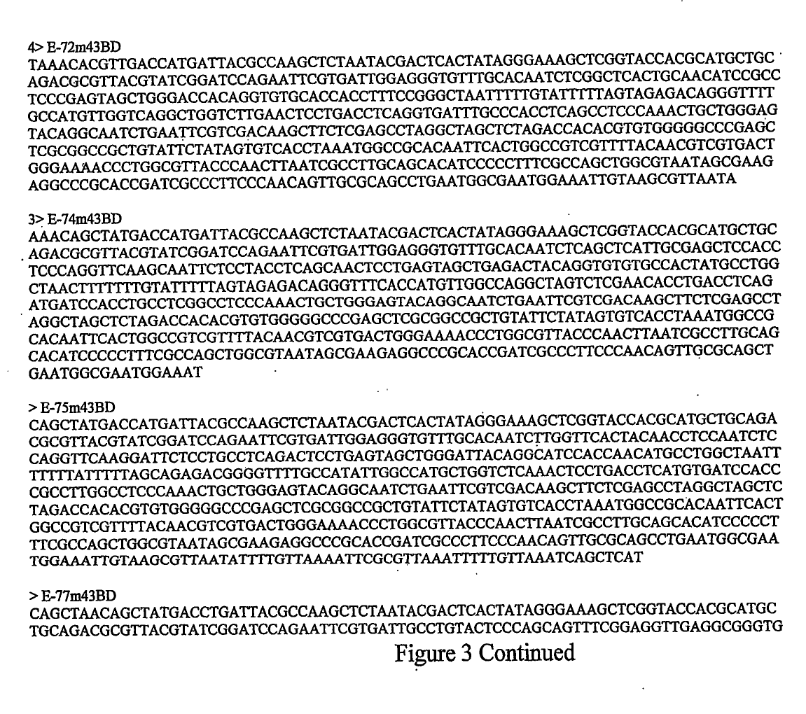

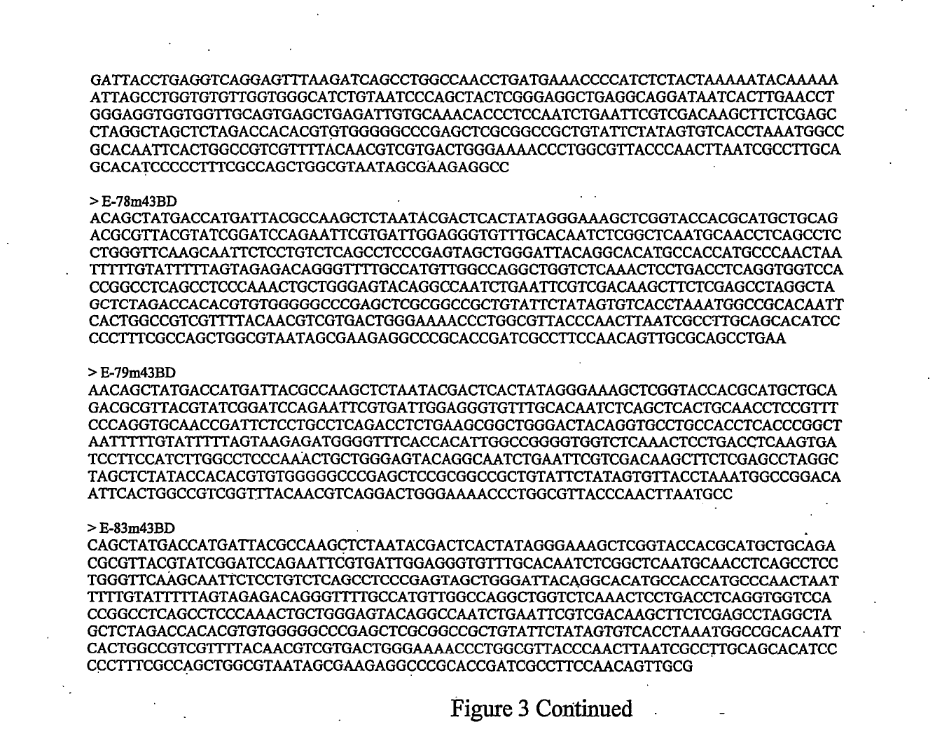

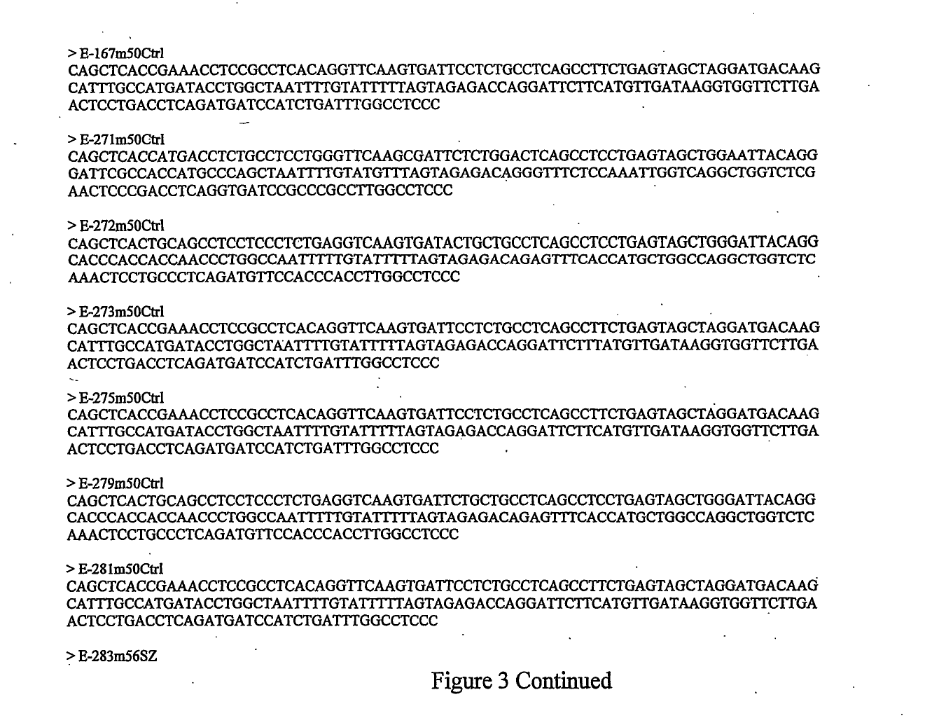

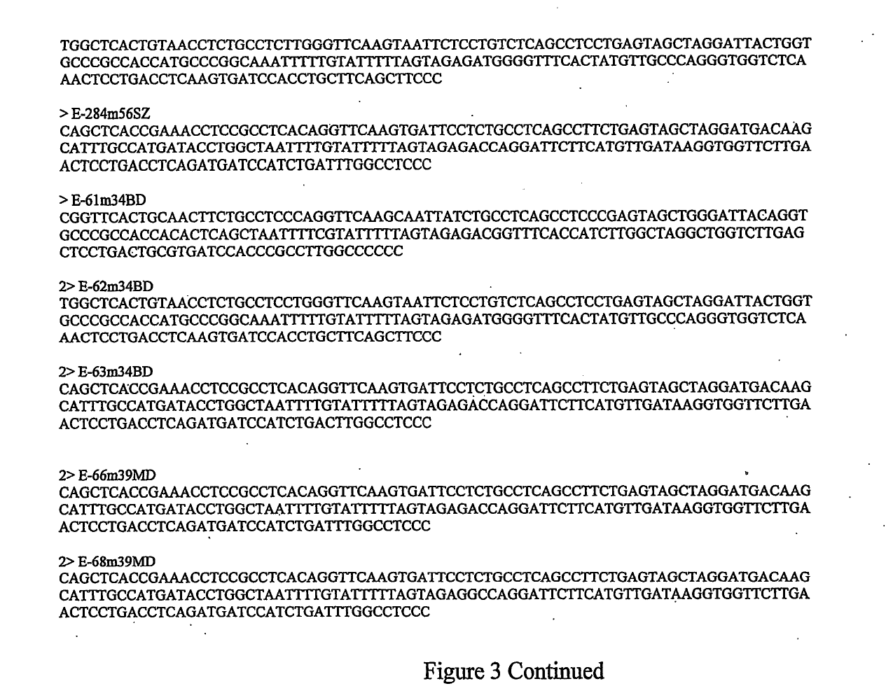

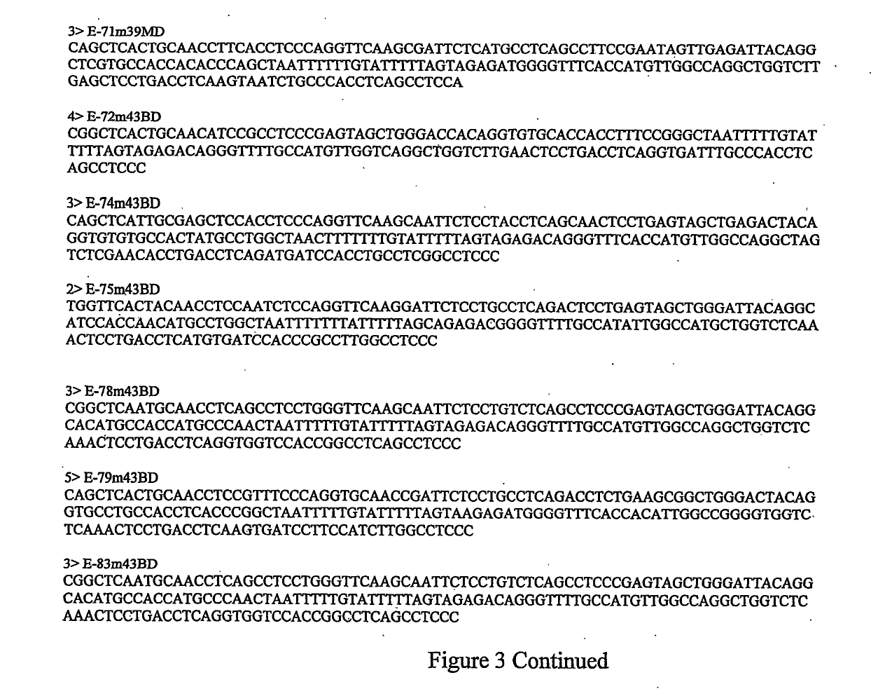

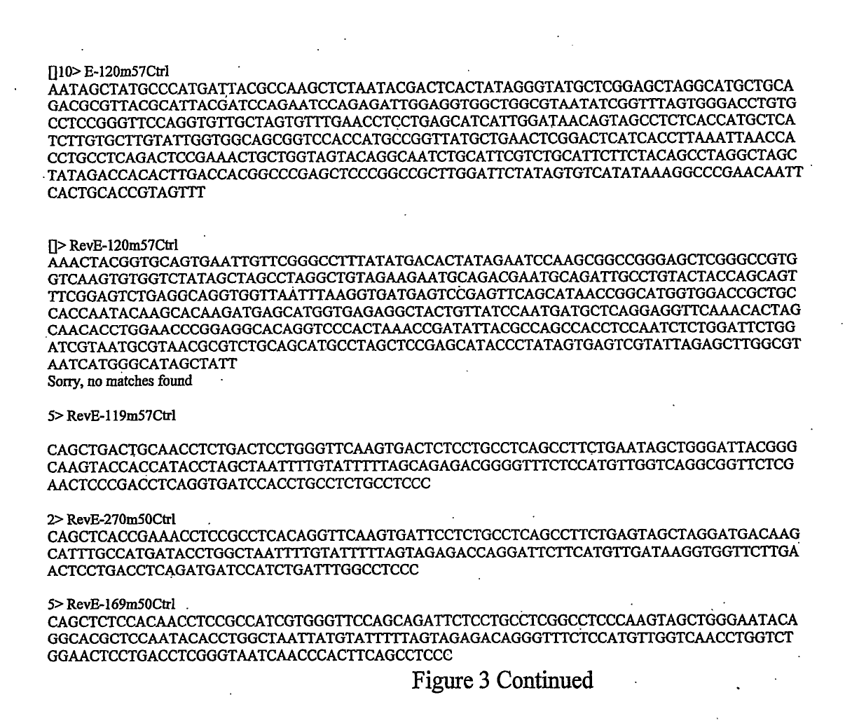

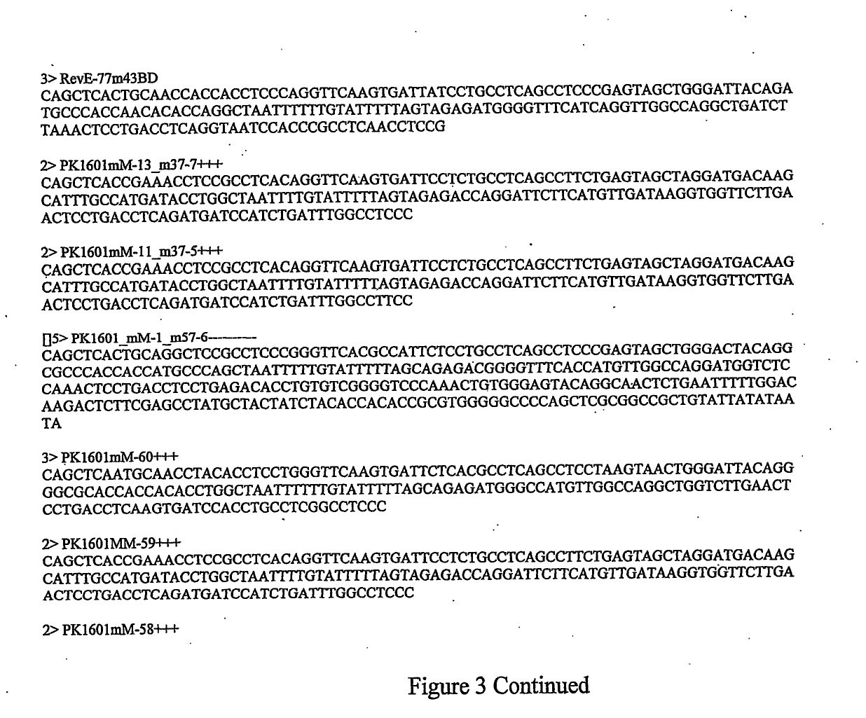

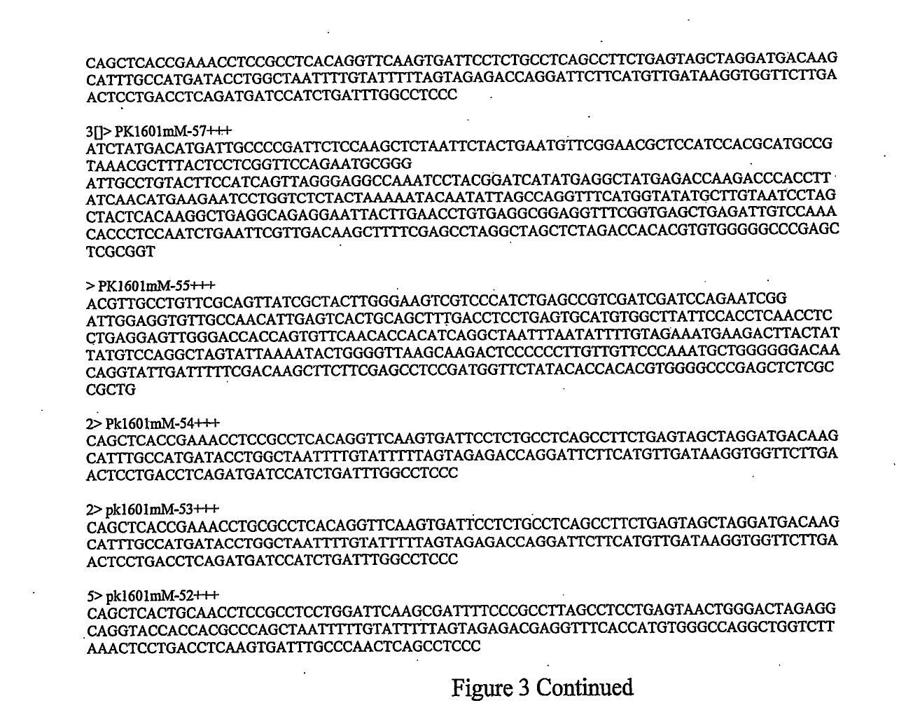

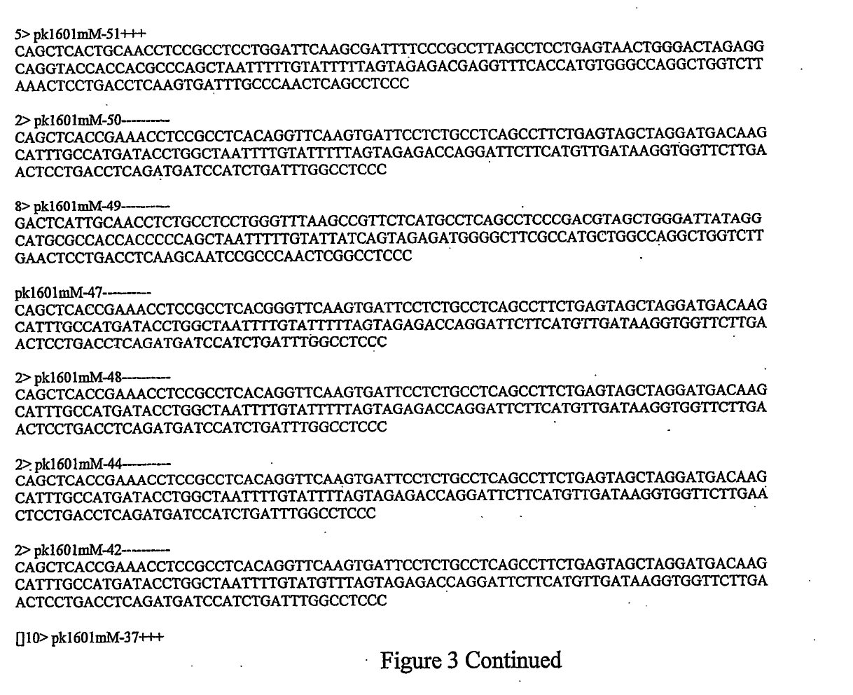

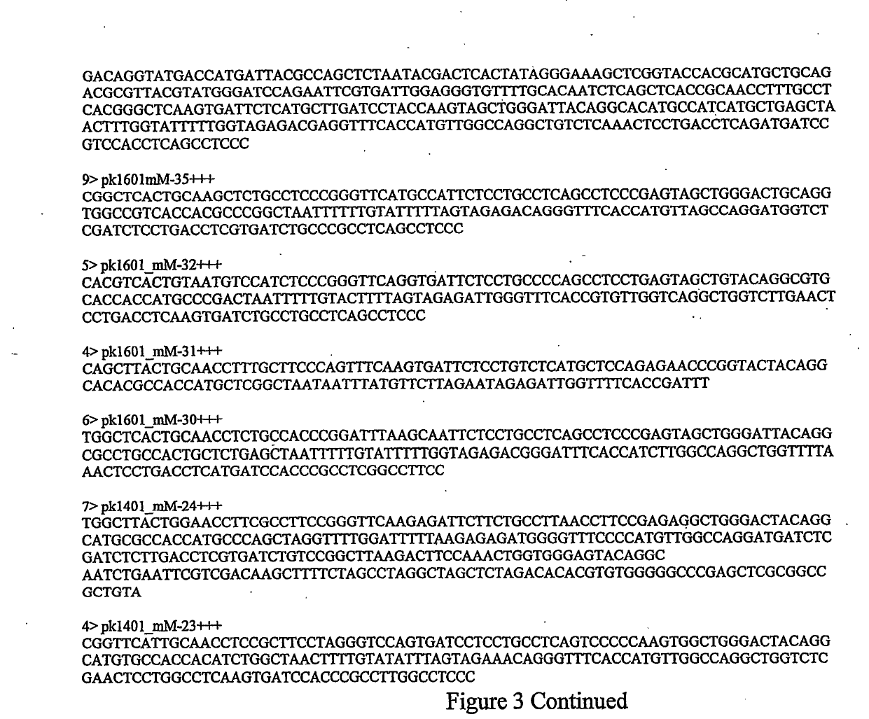

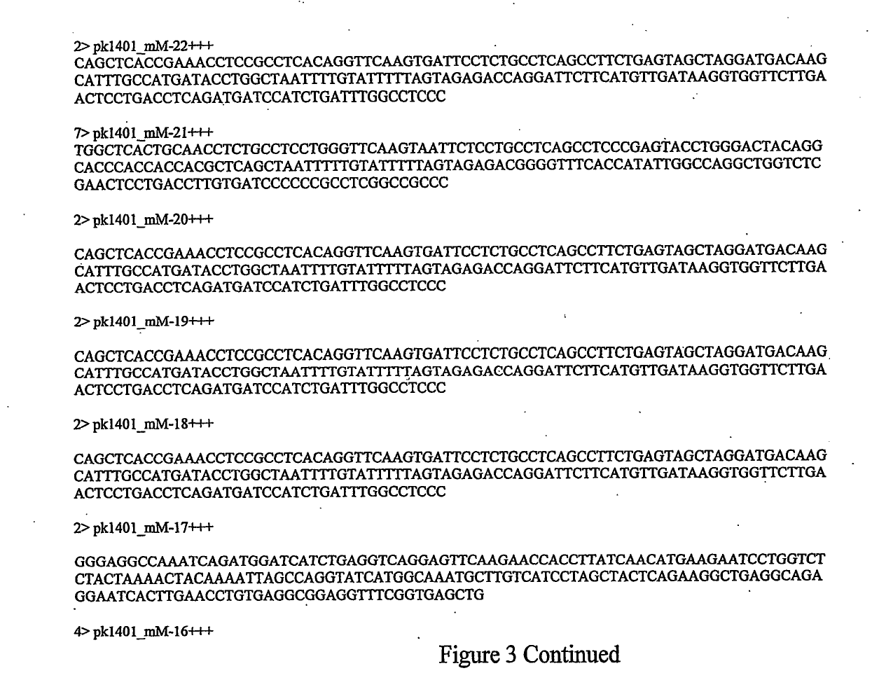

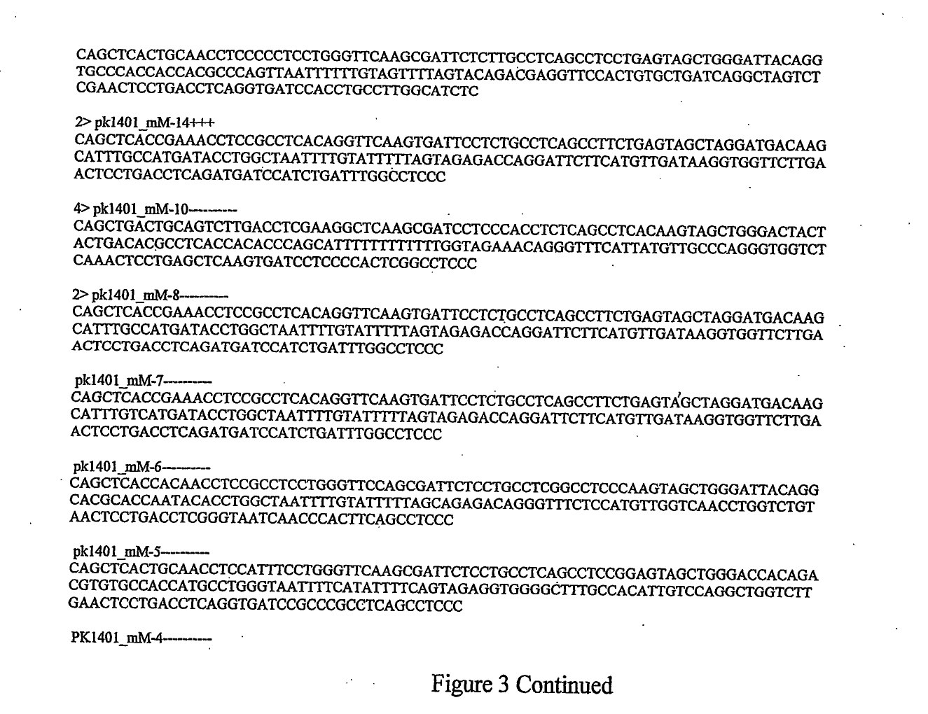

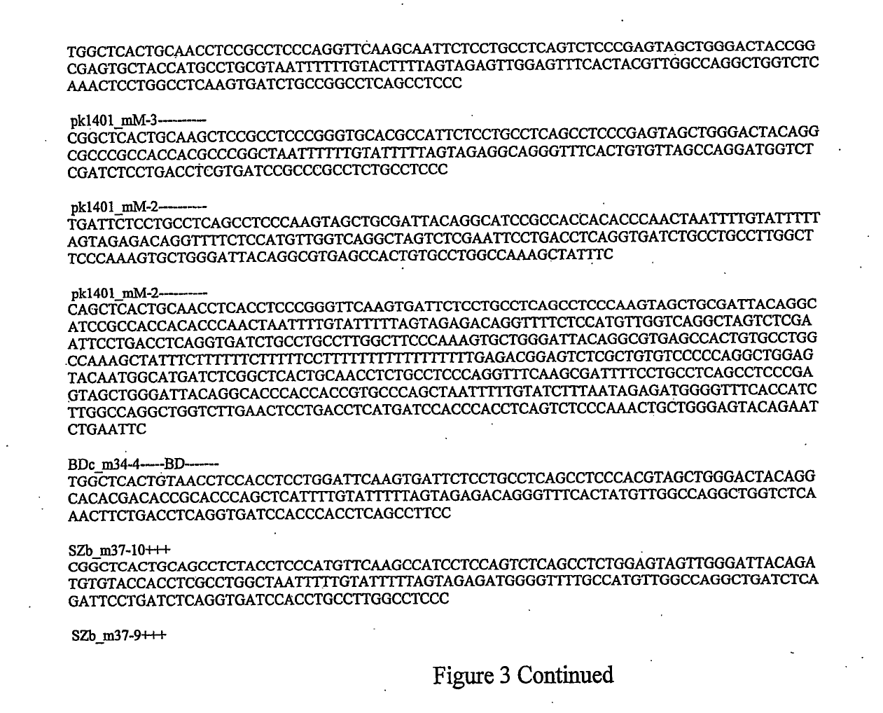

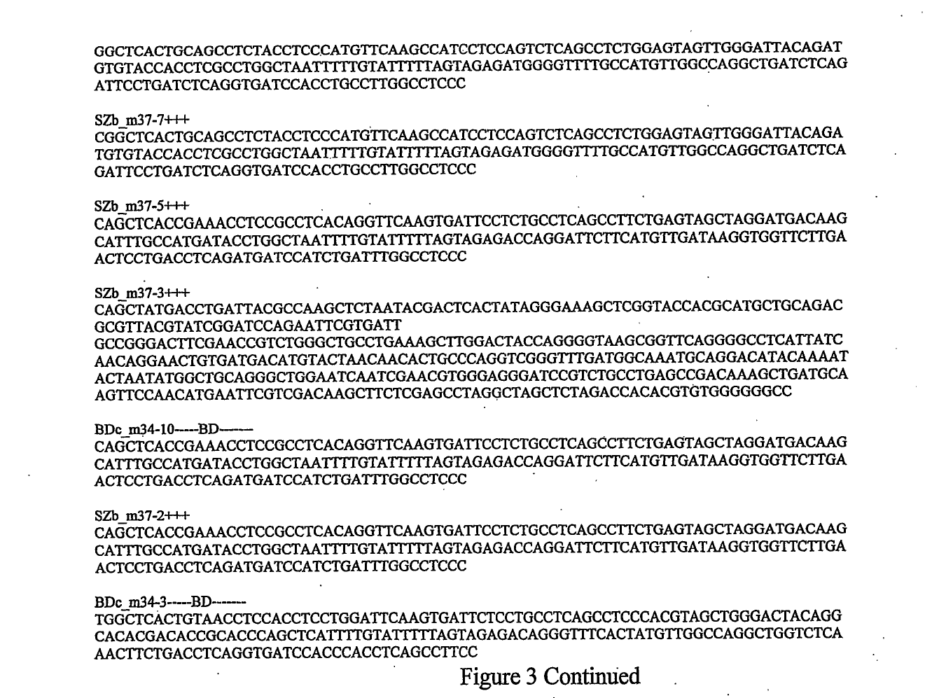

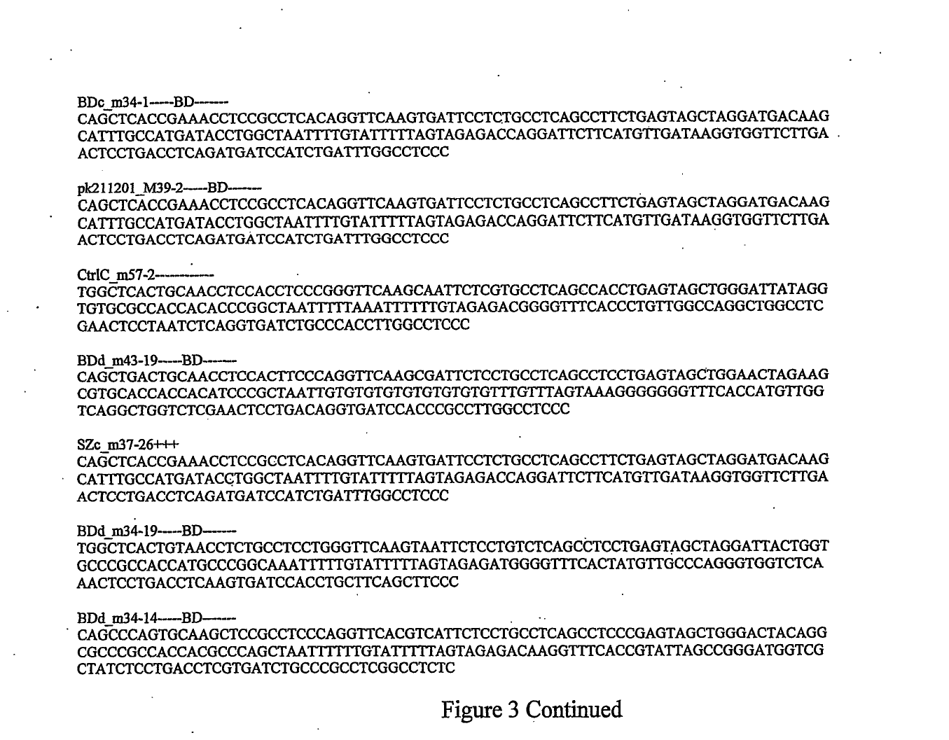

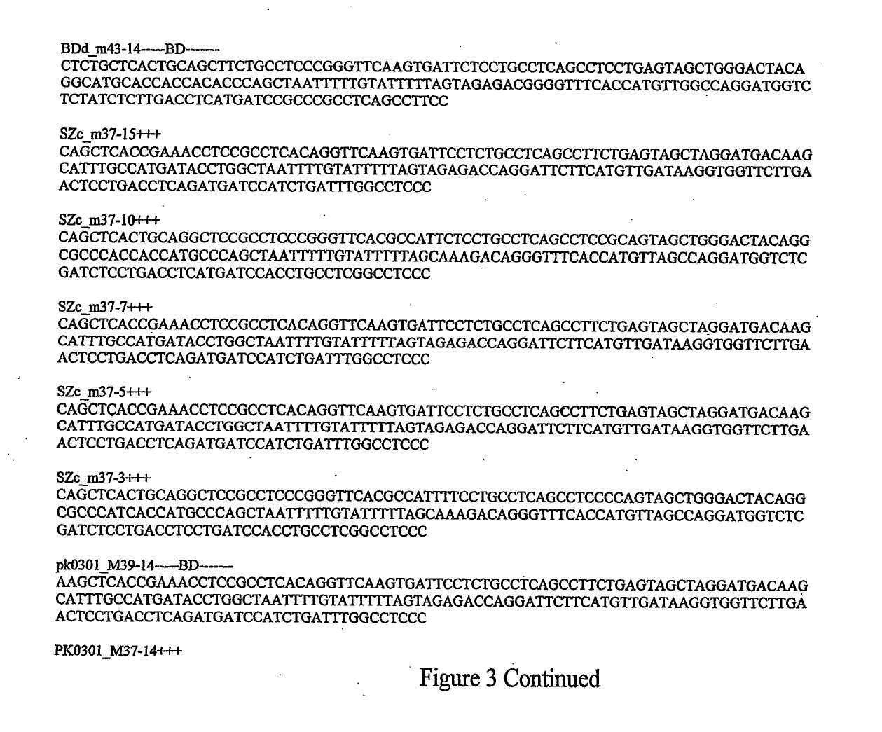

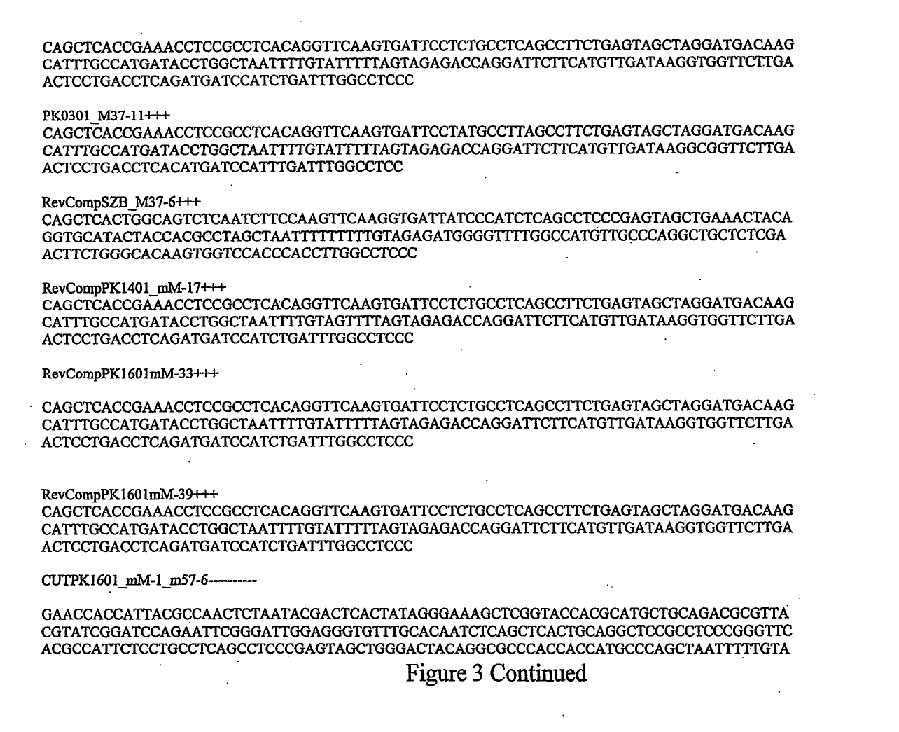

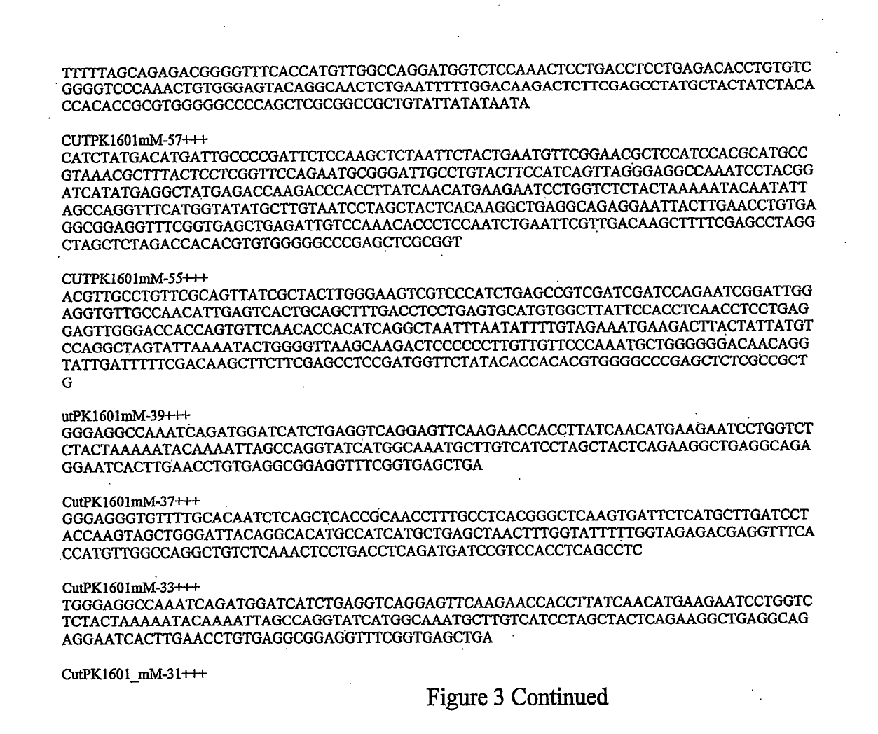

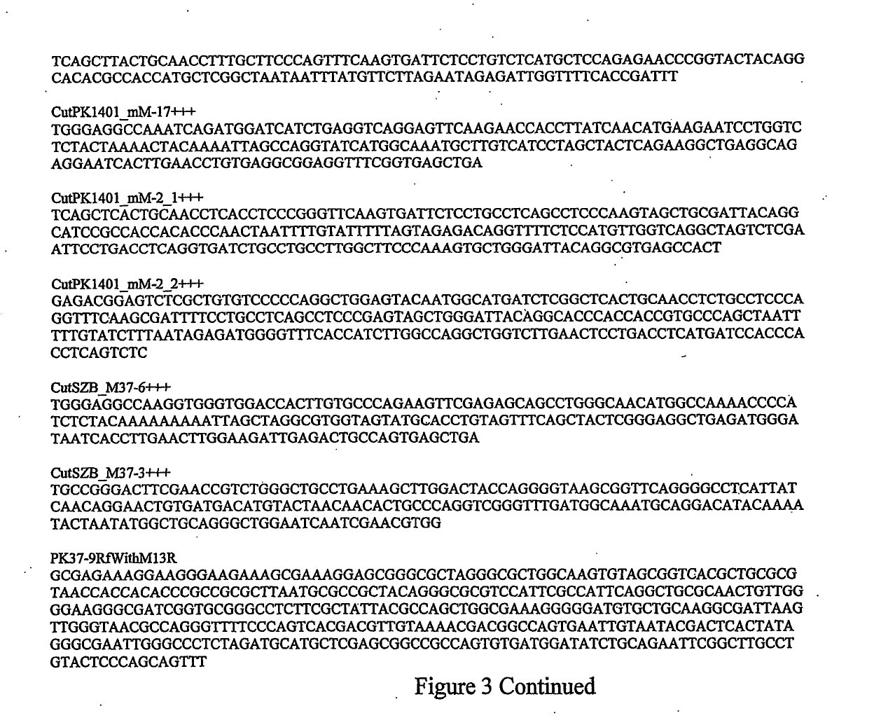

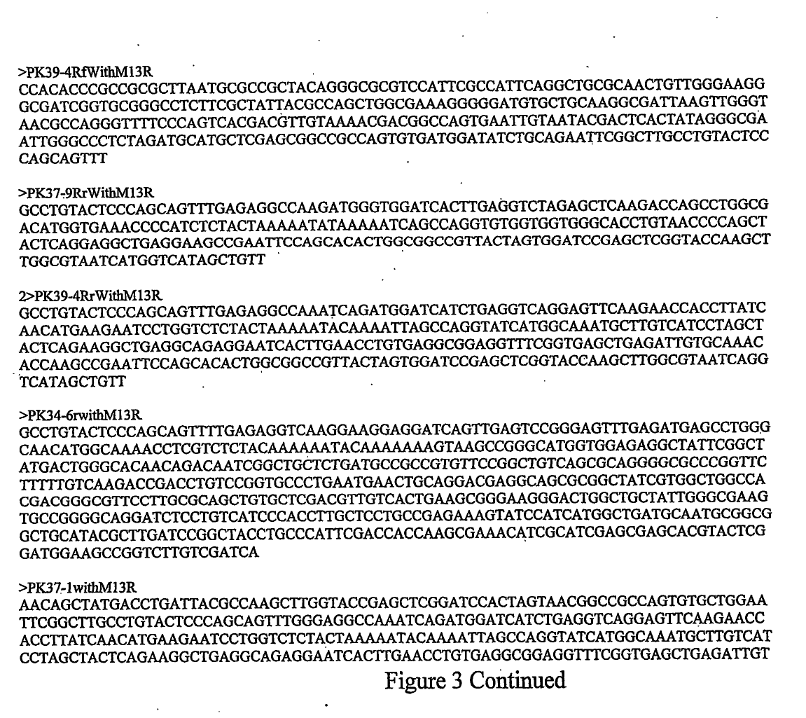

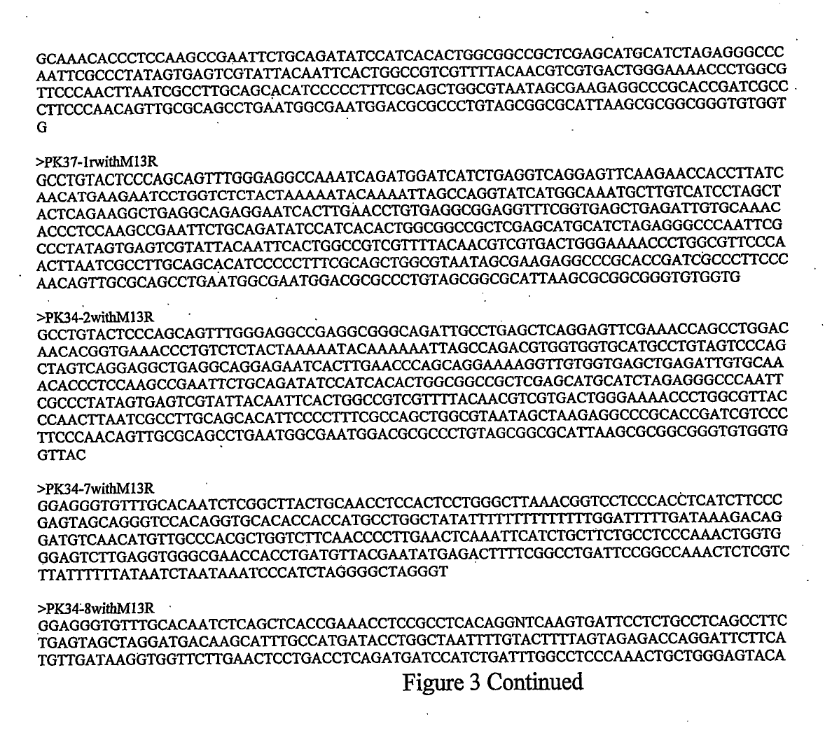

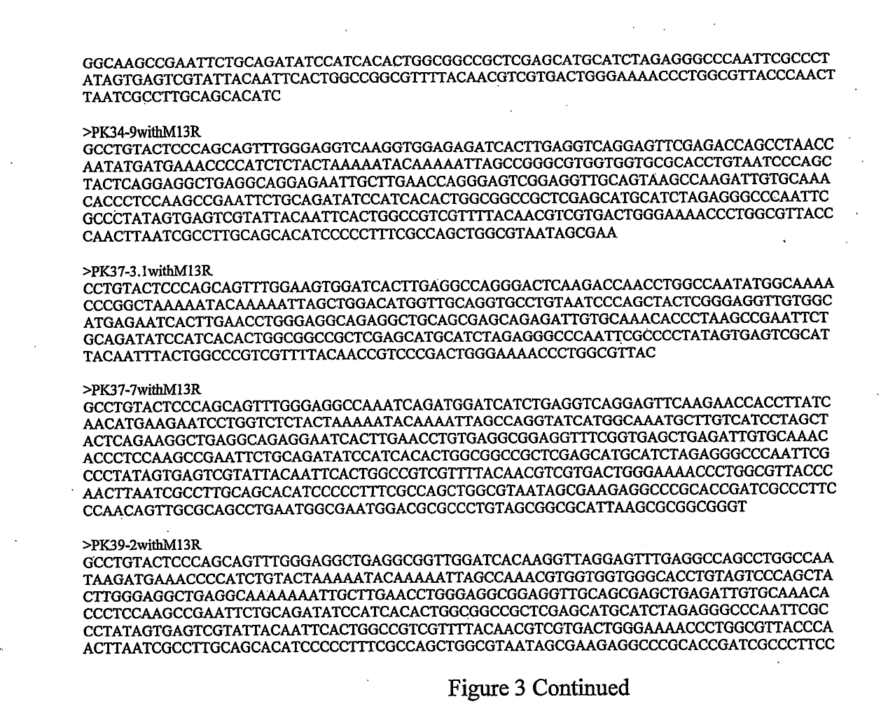

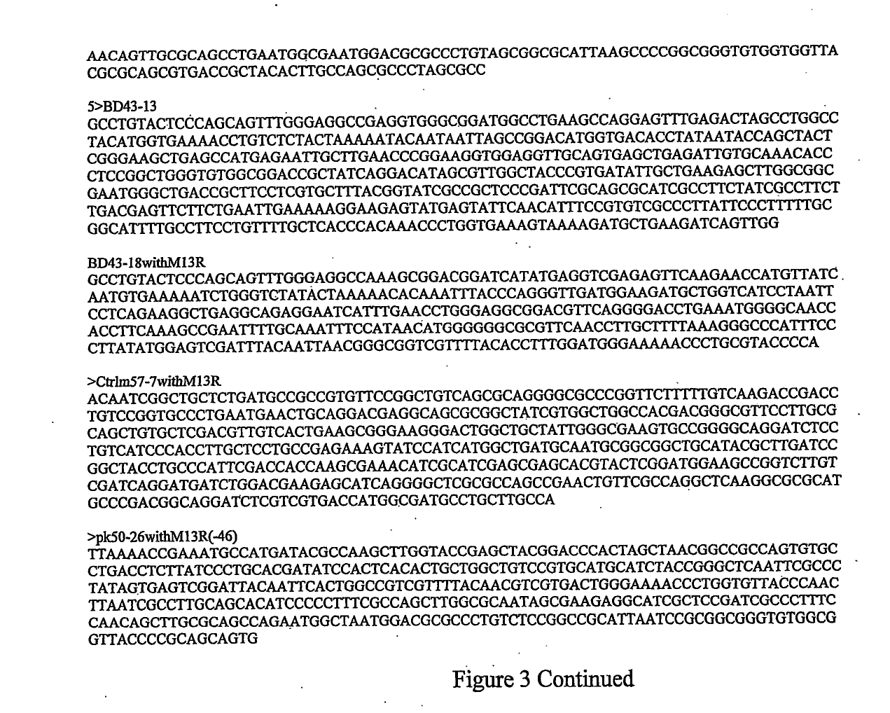

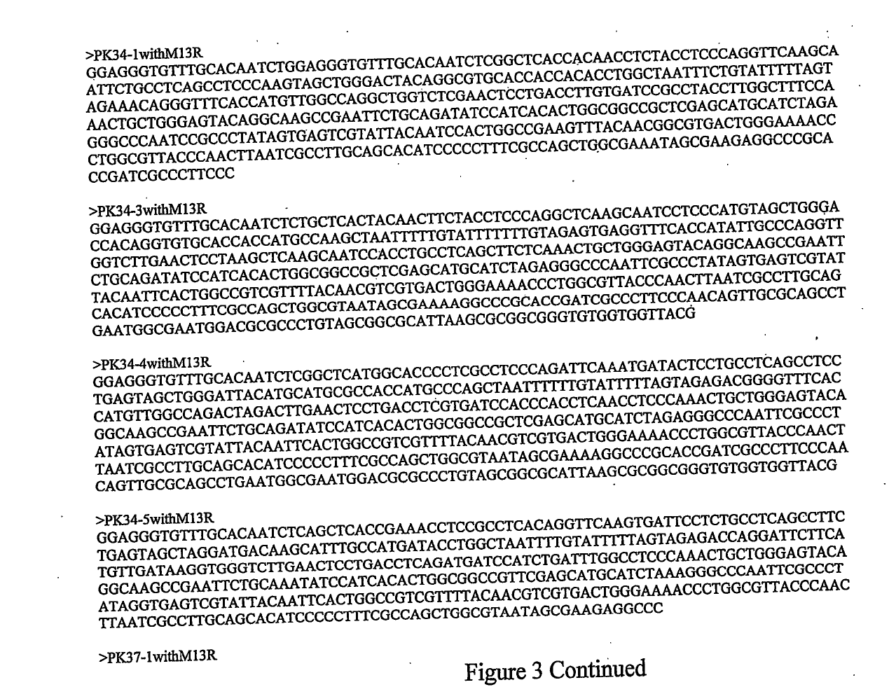

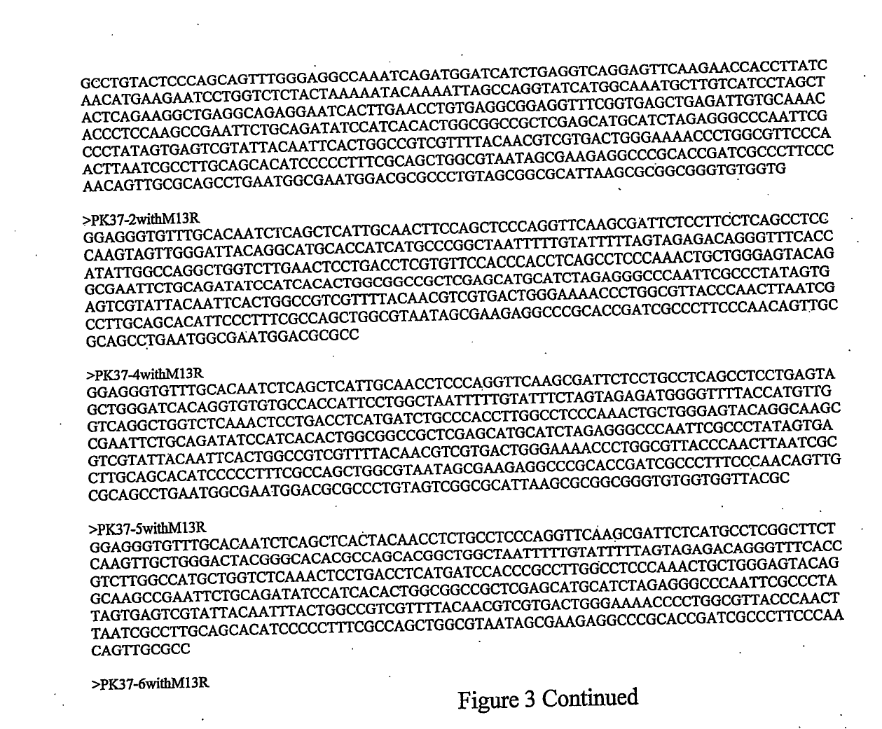

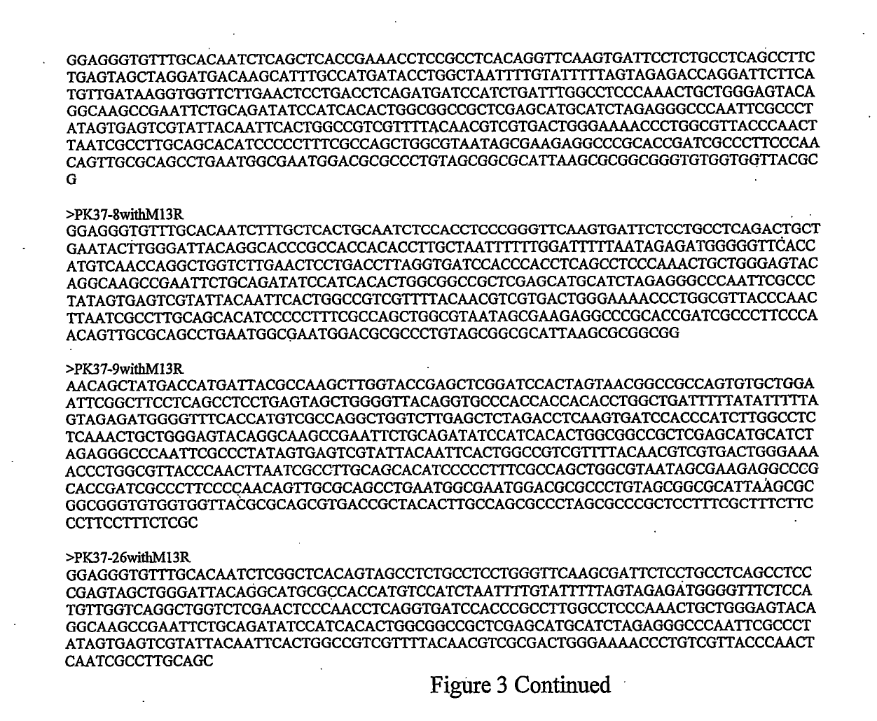

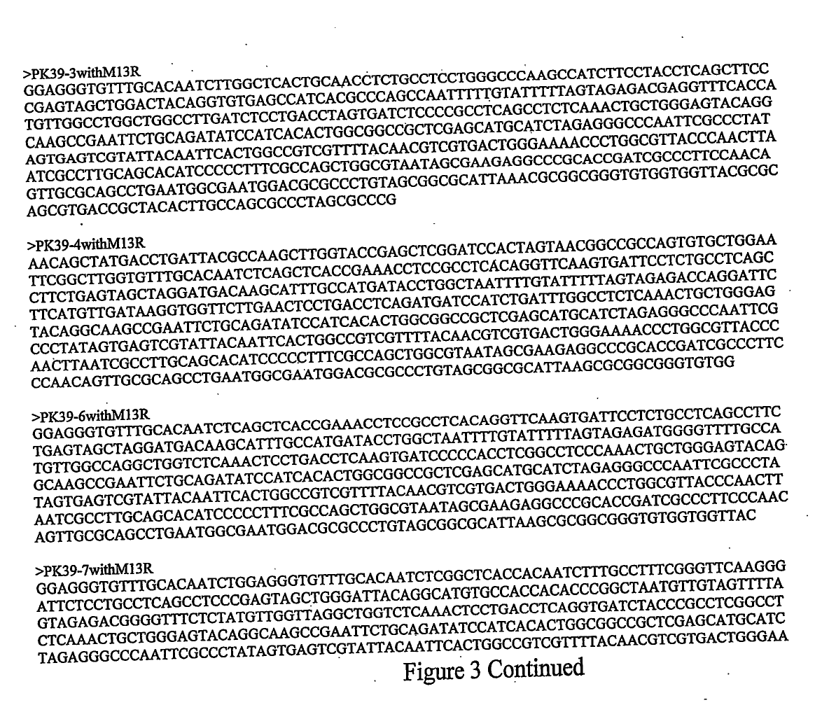

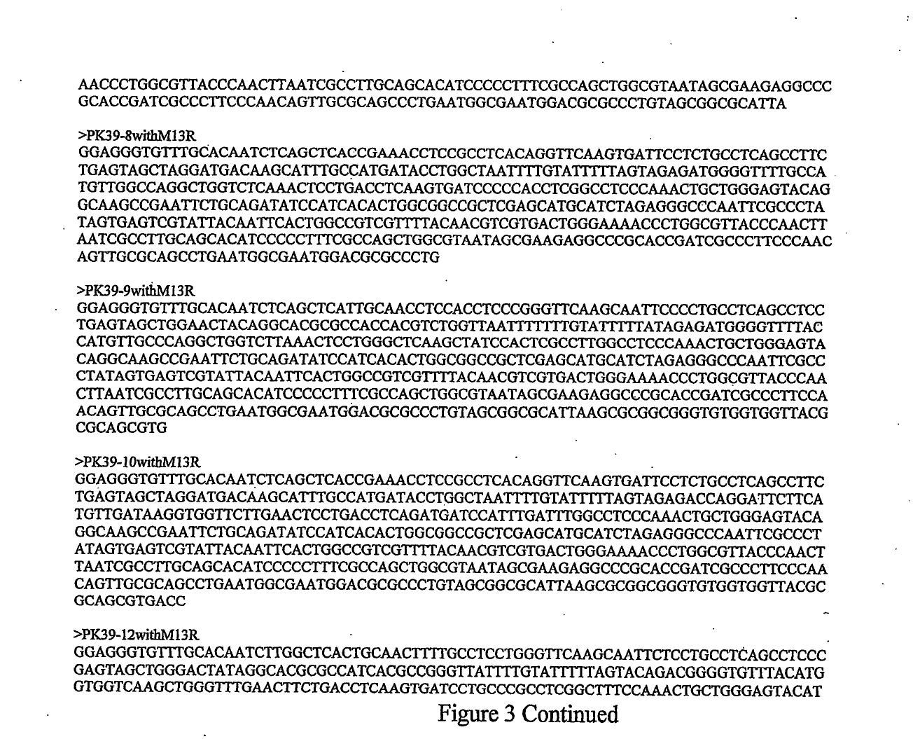

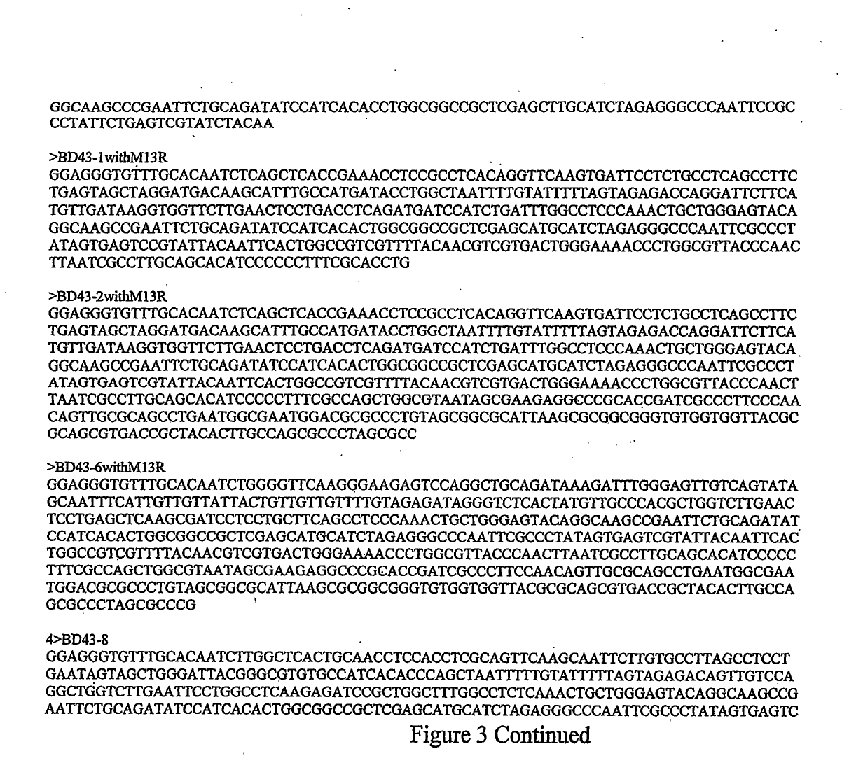

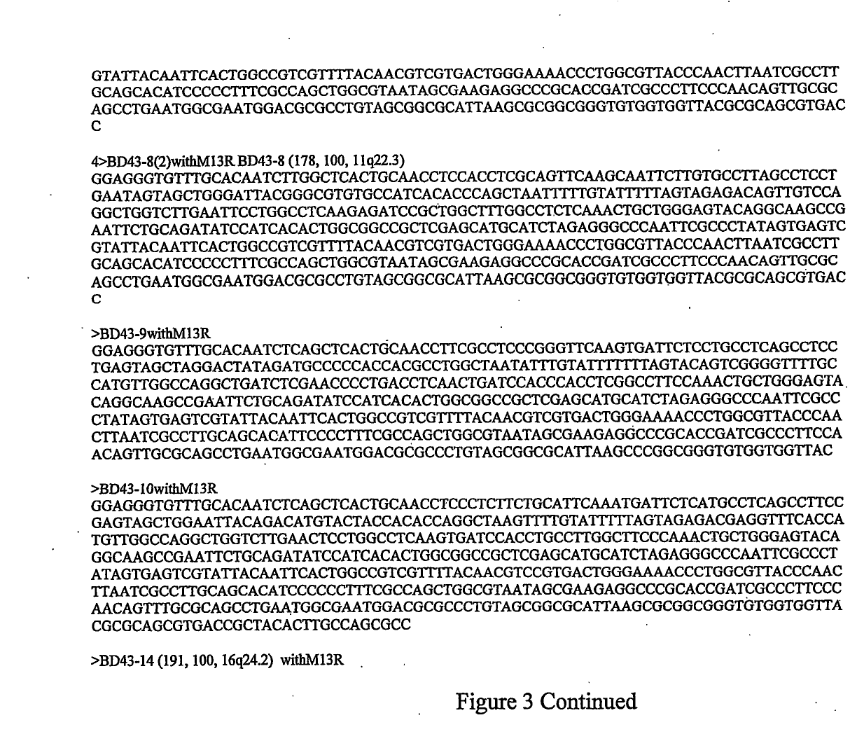

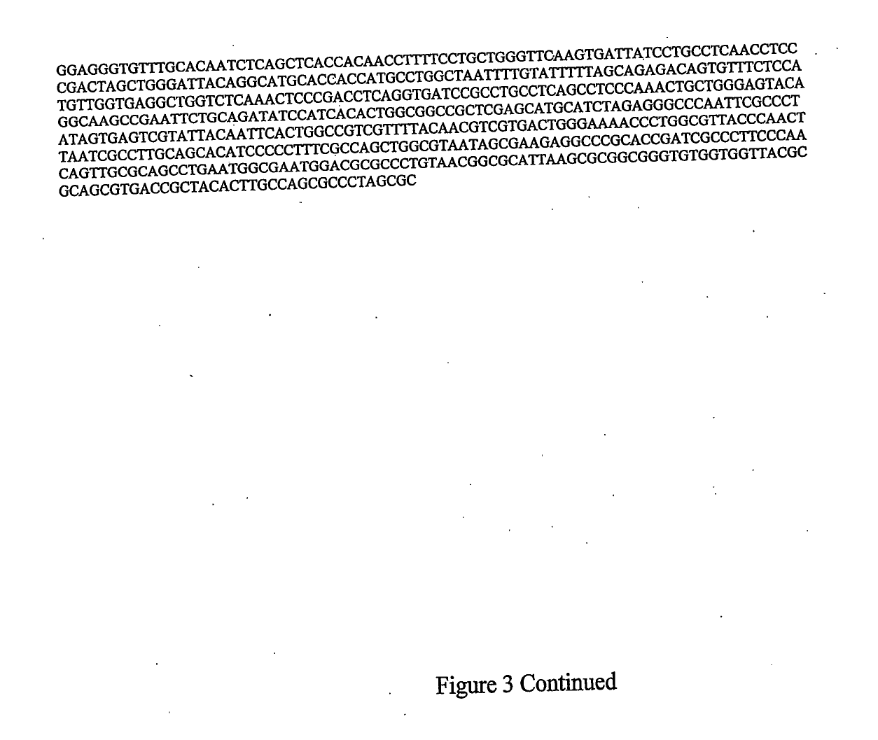

Nucleic acid sequences of amplification products isolated according to methods of the present invention are-disclosed in FIG. 3. The region of the chromosome to which a given sequence is located may be determined by hybridization, including, but not limited to PCR amplification methods, or by database searching.

Hybridization methods and conditions are well known in the art. Nucleic acids that are identical to the provided nucleic acid sequences, bind to the provided nucleic acid sequences (disclosed in FIG. 3) under stringent hybridization conditions. By using probes, particularly labeled probes of DNA sequences, one can determine a region of chromosome where a given sequence is located and thereby establish chromosomal loci for epigenetic abnormalities associated with a disease, including Mendelian or non-Mendelian disease.

Preferably, hybridization is performed using at least 15 contiguous nucleotides from any sequence identified by the methods of the present invention including, but not limited to, sequences disclosed in FIG. 3. The probe will preferentially hybridize with a nucleic acid comprising a complementary sequence to the probe, allowing the identification of the chromosomal region of the nucleic acids of the biological material that uniquely hybridize to the selected probe. Probes of more than 15 nucleotides can be used, e.g. probes of from about 18 nucleotides up to the entire length of the provided nucleic acid sequences, but 15 nucleotides generally represents sufficient sequence for unique identification.

As mentioned above once the sequence (or a portion of the sequence) of a multi-copy DNA element has been isolated, this sequence can be used to map the location of the multi-copy DNA element on a chromosome. Accordingly, nucleic acids of the invention described herein or fragments thereof, can be used to map the location of multi-copy DNA elements of the invention on a chromosome. The mapping of the sequences of nucleic acids of the invention to chromosomes is an important first step in correlating these sequences with genes associated with disease.

Briefly, sequences of the invention, for example, sequences disclosed in FIG. 3, can be mapped to chromosomes by preparing PCR primers (preferably 15-25 bp in length) from the sequences of nucleic acids of the invention. These primers can then be used for PCR screening of somatic cell hybrids containing individual human chromosomes. Only those hybrids containing the human sequence corresponding to the sequences of nucleic acids of the invention will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow (because they lack a particular enzyme), but in which human cells can, the one human chromosome that contains the gene encoding a needed enzyme, depending on the media, will be retained. By using various media, panels of hybrid cell lines can be established. Each cell line in a panel contains either a single human chromosome or a small number of human chromosomes, and a fall set of mouse chromosomes, allowing easy mapping of individual sequences to specific human chromosomes. (D'Eustachio et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the sequences of nucleic acids of the invention to design oligonucleotide primers, sublocalization can be achieved with panels of fragments from specific chromosomes. Other mapping strategies which can similarly be used to map a sequence of a nucleic acid of the invention to its chromosome include in situ hybridization (described in Fan et al. (1990) Proc. Natl. Acad. Sci. USA 87:6223-27), pre-screening with labeled flow-sorted chromosomes, pre-selection by hybridization to chromosome specific cDNA libraries, and searching of genomic databases.

Of course, persons skilled in the art will recognize that actual physical mapping of a multi-copy DNA element on a chromosome, as described above, may not be necessary where the multi-copy DNA element can be mapped in silico.

Once the sequence (or a portion of the sequence) of a multi-copy DNA element has been isolated, this sequence can be used to map the location of the gene on a chromosome by searching a genomic database, for example, but not limited to, a human genome database (www.genome.ucsc.edu/). Several genome databases are also available from Celera Corp. or the National Center for Biotechnology Information (NCBI). Genome databases can be searched by comparing the known query sequence or reference sequence with genomic sequences stored and annotated in a database, and selecting sequences from the database that have a high similarity, preferably greater than 80% similarity, with the query or reference sequence. Sequence similarity is calculated based on a reference sequence, which may be a subset of a larger sequence, such as a conserved motif, coding region, flanking region, etc. A reference sequence will usually be at least about 18 contiguous nucleotides long, more usually at least about 30 nucleotides long, and may extend to the complete sequence that is being compared. Algorithms for sequence analysis are known in the art, such as BLAST, described in Altschul et al., J. Mol. Biol. (1990) 215:403-10.

To determine whether a nucleic acid exhibits similarity with the sequences presented herein, oligonucleotide alignment algorithms may be used, for example, but not limited to a BLAST (GenBank URL: www.ncbi.nlm.nih.gov/cgi-bin/BLAST/, using default parameters: Program: blastn; Database: nr; Expect 10; filter: default; Alignment: pairwise; Query genetic Codes: Standard(1)), BLAST2 (EMBL URL: http://www.embl-heidelberg.de/Services/index.html using default parameters: Matrix BLOSUM62; Filter: default, echofilter: on, Expect:10, cutoff: default; Strand: both; Descriptions: 50, Alignments: 50), or FASTA, search, using default parameters.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical, e.g., colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., (Human Chromosomes: A Manual of Basic Techniques (Pergamon Press, New York, 1988)). Sequences of isolated multi-copy DNA elements of the present invention that are shorter than 500 bases can be extended by any suitable technique, for example, a known sequence can be extended by a technique of genomic sequencing using a primer designed according to the known sequence.

Reagents for chromosome mapping can be- used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature 325: 783-787.

Probes specific to the nucleic acids of the invention can be generated using a whole or portion of the nucleic acid sequences disclosed in FIG. 3. The probes can be synthesized chemically or can be generated from longer nucleic acids using restriction enzymes. The probes can be labeled, for example, with a radioactive, biotinylated, or fluorescent tag. Preferably, probes are designed based upon an identifying sequence of a nucleic acid of one of FIG. 3. More preferably, probes are designed based on a contiguous sequence of one of the subject nucleic acids that remain unmasked following application of a masking program for masking low complexity (e.g., XBLAST) to the sequence., i.e. one would select an unmasked region, as indicated by the nucleic acids outside the poly-n stretches of the masked sequence produced by the masking program. Probes are not only useful for determining chromosomal location of a sequence, but also can be used to determine whether an epigenetic abnormality exists in another sample, for example a test sample obtained from a eukaryotic organism that exhibits symptoms of a disease, including Mendelian or non-Mendelian disease.

Once a chromosomal locus has been assigned to a multi-copy DNA element obtained by the present invention, a genomic database or genetic map data can be used to identify one or more genes, for example about 1 to about 10 genes, that are proximal to the assigned chromosomal locus, preferably the identified one or more genes are physically adjacent to the assigned locus. Expression patterns of the genes in a Mendelian or non-Mendelian disease sample can then be compared against the expression pattern of corresponding genes in a control sample to identify a gene having an epigenetically altered expression pattern. The disease sample and the control sample can be obtained from within the same organism, for example, without wishing to be limiting, expression of a gene within cancerous kidney cells could be compared against expression of a corresponding gene in a non-cancerous kidney cell of the same organism. Alternately, the disease sample and the control sample can be obtained from different organisms. For example, without wishing to be limiting, expression of a gene in a prefrontal cortex sample from a schizophrenic individual can be compared against expression of a corresponding gene in a prefrontal cortex sample from a different non-schizophrenic individual. As another example, expression of a gene in a cerebellum sample from a Huntingdon's disease patient can be compared against expression of a corresponding gene in a cerebellum sample obtained from a subject not suffering from Huntingdon's disease.

Techniques for determining expression patterns of genes are well known in the art. For example, gene expression patterns can be established using Northern analysis, reporter constructs such as GFP, quantitative PCR amplification, or DNA chip analysis (microarrays). If, for example, gene expression within a sample is determined using DNA chips, the mRNA from the sample is extracted, reverse transcribed to the corresponding cDNA, amplified, fluorescently labeled and allowed to hybridize with the sequences on a chip. Sequence-specific labels are captured on the surface of the chip. By reading the fluorescence, one can determine which of the genes were expressed and at what levels. DNA chip analysis is provided by several companies, for example, but not limited to, Affymetrix and Nanogen. DNA chip technology is an effective method for determining expression patterns of genes and semiconductor fabrication technology has allowed for the packing of thousands of gene sequences into square centimeter surfaces. Use of reporter constructs, Northern analysis, and quantitative PCR amplification are equally effective alternatives.

Potential Therapeutic Approaches.

Detection of epigenetic abnormalities associated with diseases including, but not limited to schizophrenia, diabetes, cancers, bipolar disorder, cystic fibrosis, Duchennes muscular dystrophy, Huntington's disease and fragile X syndrome, may lead to innovative DNA modification-based therapies. Recently a compound protein consisting of a DNA methylation enzyme and a zinc-finger protein was constructed (Xu G-L, Bestor T H. Nature Genetics 17: 376-379, 1997). The mechanism of action of the protein consists of the recognition of a specific DNA sequence by the zinc-finger protein that is specific for that sequence and subsequent modification of the surrounding cytosines by DNA modification enzymes. A specific protein with DNA modification enzyme restoring the normal pattern of DNA methylation can be generated. The blood-brain barrier has been a major obstacle for the bloodborne genetic constructs to reach the brain, but a recent study demonstrated that pegylated neutral liposomes, unlike cationic ones, are stable in blood, do not get entrapped in the lung, and are able to efficiently deliver plasmid DNA through the blood brain barrier to the various sections of brain tissue.

The present invention provides methods and compositions for detecting DNA elements that act as a marker for the specific dysfunctional genes and at the same time identify the specific genes involved in diseases. Such information would lead quickly to the development of a diagnostic test for such diseases, that could be incorporated into a diagnostic kit. Further research on specific genes may also lead to treatment options for people suffering from-disease through either gene therapy work or through targeted drug development.

The heuristic value of epigenetics in diseases, including schizophrenia, derives from numerous important characteristics of epigenetic regulation of genes (Petronis A. Human morbid genetics revisited: relevance of epigenetics. Trends Genet. March 2001; 17(3):142-6). The epigenetic research program indicates that regulation of gene activity is critically important for normal functioning of the genome. Genes, even the ones that carry no mutations or disease predisposing polymorphisms, may be useless or even harmful if not expressed in the appropriate amount, at the right time of the cell cycle, or in the right compartment of the nucleus. Epigenetic mechanisms, more so than DNA sequence-based ones, can explain a series of phenomenological features of a non-Mendelian disease, for example, in the case of, major psychosis including: i) relatively late age of onset and coincidence of the first symptoms with changes in the hormonal status in the organism; ii) sexual dimorphism; iii) fluctuating course and sometimes recovery; iv) parental origin effects; and v) discordance of MZ twins. Furthermore, re-analysis of several etiological theories of major psychosis from an epigenetic point of view (Petronis A, Paterson A D, Kennedy J L. Schizophrenia: an epigenetic puzzle? Schizophrenia Bulletin 25:4: 639-655, 1999; Petronis A. The genes for major psychosis: aberrant sequence or regulation? Neuropsychopharmacology, 23(1): 1-12; 2000) suggested that epigenetic mechanisms have the potential to explain a number of clinical and molecular findings that traditionally have been supporting unrelated and somewhat antagonistic theories of schizophrenia and bipolar disorder, or have not been explained at all. Epigenetic dysfunction may exhibit stability during meiosis and therefore can be transmitted from one generation to another (Klar A J. Propagating epigenetic states through meiosis: where Mendel's gene is more than a DNA moiety. Trends Genet 1998; 14(8):299-301; Cavalli G, Paro R. The Drosophila Fab-7 chromosomal element conveys epigenetic inheritance during mitosis and meiosis. Cell 1998; 93(4):505-18; Allen N D, Norris M L, Surani M A. Epigenetic control of transgene expression and imprinting by genotype-specific modifiers. Cell Jun. 1, 1990;61(5):853-61; Silva A J, White R. Inheritance of allelic blueprints for methylation patterns. Cell Jul. 15, 1988; 54(2):145-52; Morgan H D, Sutherland H G, Martin D I, and Whitelaw E (1999) Epigenetic inheritance at the agouti locus in the mouse. Nature Genetics 23: 314-8), which would simulate familial, i.e. genetic, cases of the disease.

The above description is not intended to limit the claimed invention in any manner. Furthermore, the discussed combination of features might not be absolutely necessary for the inventive solution.

The present invention will be further illustrated in the following examples. However, it is to be understood that these examples are for illustrative purposed only, and should not be used to limit the scope of the present invention in any manner.

### EXAMPLES

### Example 1

### Identification of Loci Having a Hypomethylated Sequence and a Retroelement in Schizophrenia or Bipolar Disorder

Brain tissues. Prefrontal cortex from post-mortem brains of individuals who were affected with various psychiatric disorders (N=39; age at death [+S.D.] 40+12 yr) and controls (N=9; age at death 48+7 yr) were subjected to analysis. In the affected group, there were 26 males and 13 females, and the controls consisted of 8 males and 1 female. The distribution of psychiatric diagnoses was as follows: 11 bipolar disorder, 9 schizophrenia, 11 non-psychotic depression, and 8 psychosis NOS. The overwhelming majority of the tested samples were from Caucasians, 1 American Black, and 2 Asians (all three affected). Brain tissues were kindly provided by the Stanley Foundation Brain Bank.

Methods. DNA samples were extracted from the brain tissues using a standard phenol-chloroform extraction technique. Before the digestion of genomic DNA with a methylation sensitive restriction enzyme, an additional step of separation of the high molecular weight DNA (>15-20 kb) from the partially degraded DNA was performed. The degraded DNA was removed by fractionation of 15 microgram of undigested genomic DNA on a 1% low melting point agarose gel (Promega), cutting the agarose block that contained high molecular weight (>15-20 kb) DNA, and incubating the block with an agarose- digesting enzyme, agarase, as recommended by the manufacturer (MBI Fermentas). After the agarose blocks were completely digested, the high molecular weight DNA samples were digested with 50 units of methylation sensitive restriction enzyme, HpaII (MBI Fermentas) overnight. A test experiment using phage lambda DNA showed that the products of the agarase-treated agarose did not affect the ability of the restriction enzyme to cut DNA. In the next step, the unmethylated fraction of brain specific DNA was separated from the hypermethylated fraction of DNA using a similar, gel-electrophoresis-based approach, during which DNA fragments smaller than arbitrarily selected 4 kb were cut out from the gel, purified using the NucleoSpin Extraction Kits (Clontech), and dissolved in 30 microliter of water. One to two microliter of the hypomethylated DNA solution were screened for the presence of-Alu sequences.

Alu sequences were sought using a protocol similat to the nested PCR protocol as in (Karlsson et al 2001) with primers that match the Alu sequences. Alu primer sequences were ‘Alu For’ GCCTGTACTCCCAGCAGTTT (SEQ ID NO:2) and ‘Alu Rev’ GGAGGGTGTTTGCACAATCT (SEQ ID NO:3). The reaction was performed in 25 ul containing the standard PCR buffer, the two primers, 3 mM MgCl₂, 0.1 mM of dNTP, and 1U of Taq: Pfu polymerases mix (9:1). DNA template was denatured for 4 min at 94° C. and amplification was performed in 30 cycles at 94° C., 58° C., and 72° C., 20 seconds each step. Alu PCR products were approximately 230 bp long.

PCR generated amplicons were cloned using the Qiagen PCR Cloningplus Kit. White E. coli colonies were grown up overnight, and plasmids were extracted using the QIAprep Spin Miniprep Kit (Qiagen), and subjected to automated sequencing on the Perkin-Elmer/ABI 373A Sequencer (Automated DNA Sequencing Facility, York University, Toronto, Ontario).

The genomic location of the cloned sequences was identified using the UCSC Human Genome Project Working Draft, April 2002 assembly (http://genome.ucsc.edu/).

TABLE 1
The DNA samples that were selected for cloning
and sequencing of individual Alu's.

| Sample # | Age | Sex | Ethnic background | Diagnosis |
|---|---|---|---|---|
| 34 | 48 | F | Caucasian | Bipolar Disorder |
| 43 | 37 | F | Caucasian | Bipolar Disorder |
| 39 | 34 | M | Caucasian | Mood disorder NOS |
| 37 | 31 | M | Caucasian | Schizophrenia |
| 48 | 44 | M | Caucasian | Schizophrenia |
| 56 | 58 | M | Caucasian | Schizophrenia |
| 74 | 60 | M | Caucasian | Schizophrenia |
| 50 | 52 | M | Caucasian | Control |
| 57 | 44 | M | Caucasian | Control |

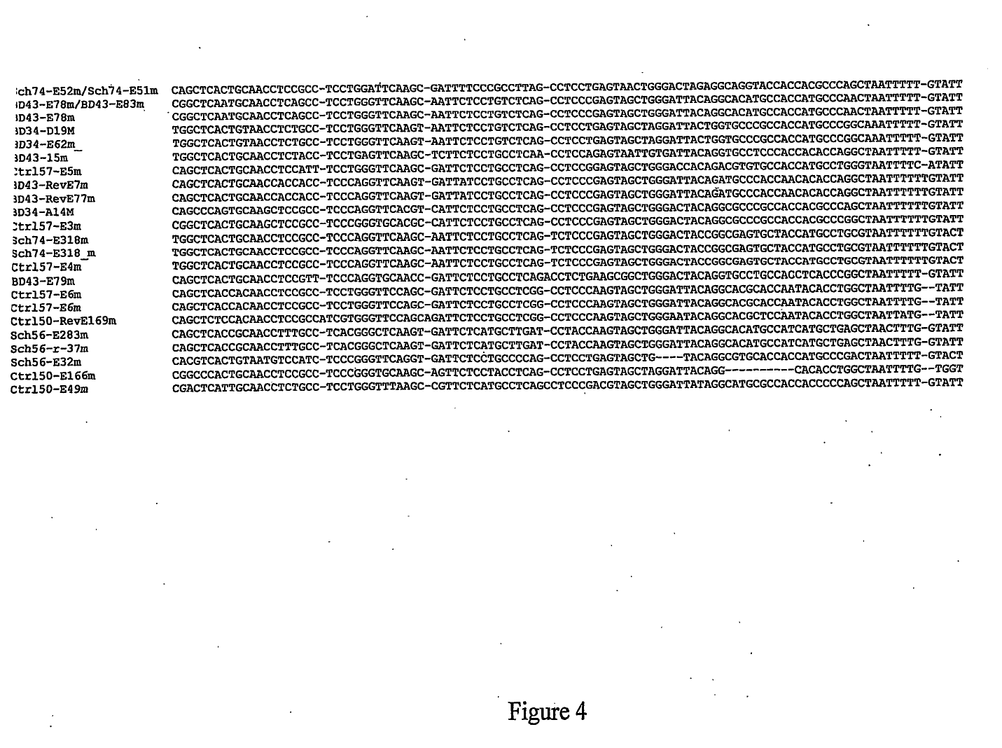

In the Alu amplification, however, agarose gel-visible (>0.1 mg) PCR fragments were produced by about half of the DNA samples after 30 PCR cycles and nearly all samples if the number of cycles was increased to 35 or 40. Nine DNA samples (Table 1) that amplified the largest amount of Alu fragments were selected for further analysis, i.e. cloning and sequencing of individual Au's. Ten to fifteen recombinant clones were sequenced from each PCR product, with a total of over 100 clones (some of these clones are presented in FIG. 4).

Genomic loci that exhibited higher than 95% of homology with the cloned Alu sequences were analyzed from two perspectives. In the first analysis, we investigated if Alu's mapped in the vicinity of known genes, and if so, how they could be related to abnormal brain functioning. The data of the Alu's mapping close to or within functional genes is presented in Table 2. About half of the Alu sequences (N=57) exhibited 100% sequence homology and mapped to Yq11.2, close to the testis transcript Y4. This indicates that the chromosome Y DNA contributed a significant portion of the hypomethylated DNA. The closest known gene to the Alu sequence on chromosome Y is the testis transcript Y4, the biological role of which is unknown. Other Alu sequences were scattered across the genome; their putative role in major. psychosis is discussed in the next section.

TABLE 2
Cloned Alu sequences located within genes
or in the close vicinity of genes

| | Homology | | |
|---|---|---|---|
| | length in | | |
| | bp; % | Chr. | |
| Clone Name | Identity | Location | Gene Name |
| BD43 -A6-m | 168 bp; 100% | 1q21 | Protein kinase, AMP- |
| | | | activated, β2 (PRKAB2) |
| | | | (31 Kb) |
| | | | KIAA1245 protein |
| BD43- | 191 bp; 99.5% | 1p31 | Densin-180 |
| RevE7m | | | |
| BD34-A14M | 187 bp; 99% | 2p23 | Brain and reproductive |
| | | | organ-expressed gene (BRE) |
| | | | (TNFRSF1A modulator)* |
| BD43-E79m | 186 bp; 96.9% | 2q37 | Leucine rich repeat |
| | | | (in FLII) interacting |
| | | | (LRRFIP1)* |
| | | | Transcriptional repressor |
| | | | (GCF2)* |
| BD43-E78m | 192 bp; 100% | 5q22 | U2 small nuclear ribo- |
| | | | nucleoprotein auxiliary |
| BD43-E83m | | | (U2AF1RS1) |
| Sch56-m32 | 189 bp; 99.5% | 6p22.3 | Ataxin 1 (SCA1)* |
| Sch37-m56 | 183 bp, 96.5% | 11q14.2 | Embryonic ectoderm |
| | | | development protein |
| | | | WAIT-1 |
| Sch74- | 192 bp; 100% | 17q12 | AIOLOS isoform two |
| E52m | | | (AIOLOS gene) (92 Kb) |
| Sch74- | | | KIAA1684 protein (6 Kb) |
| E51m | | | |
| Sch74- | 206 bp; 97.7% | 22q12 | Oncostatin M (OSM)(5 Kb) |
| E318m | | | Leukemia inhibitory factor |
| | | | (LIF)(cholinergic) (25 Kb) |
| | | | EBP50-PDZ interactor of |
| | | | 64 kD EP164 (19 Kb) |
| | | | Splicing factor 3a, 120 kD |
| | | | SF3A1 (58 Kb) |
| Numerous | 191 bp; 100% | Yq11 | Testis transcript Y 4 |
| | | | (TTY4) (90 Kb) |
| Sch and | | | HERV-K element (44 Kb) |
| BD | | | |
| clones | | | |
| Ctrl57- | 187 bp; 99% | 1q31 | Phosphatidylcholine 2- |
| E6m | | | acylhydrolase (cPLA2)* |
| Ctrl50- | 179 bp; 95% | | Calcium-dependent |
| RevE169m | | | phospholipid-binding |
| | | | protein (PLA2) |
| Ctrl50- | 185 bp; 98% | 2q36 | Potassium voltage-gated |
| E49m | | | channel, Isk-related |
| | | | KCNE4 (96 Kb) |
| Ctrl57- | 191 bp; 100% | 5q34 | WD repeat protein Gemin5* |
| E3m | | | Mitochondrial ribosomal |
| | | | protein L22 MRPL22 (18 Kb) |
| | | | CCR4-NOT transmission |
| | | | complex subunit 8 |
| | | | CNOT8 (60 Kb) |
| Ctrl57- | 188 bp; 99.0% | 13q13 | Lipoma HMGIC fusion |
| E5m | | | partner LHFP (42 Kb) |
| Numerous | 191 bp; 100% | Yq11 | Testis transcript Y4 |
| Ctrl | | | (TTY4) (90 Kb) |
| clones | | | |

Clone ID consists of disease status (Sch—schizophrenia; BD—bipolar disorder; Ctrl—control), the number of the sample, and the clone number (following the hyphen). Asterisks indicate the Alu sequences that mapped within a gene. If Alu does not map within a gene, distance to the nearest known gene is indicated in brackets (kilobases; Kb)

The second analysis investigated if the cloned Alu sequences mapped to the genomic loci that showed evidence for linkage to SCZ and BD or revealed some chromosomal abnormalities (deletions, translocations) in individuals affected with major psychosis. The data of cloned Alu sequences that match the regions of putative linkage to major psychosis are presented in Table 3. Since there is substantial overlap between the genetic loci predisposing to SCZ and the ones that increase the risk to BD (Berrettini 2000a; Berrettini 2000b; Cardno et al 2002), the type of psychosis—SCH or BD—was ignored in the matching of the cloned Alu's with the putatively linked genomic loci.

TABLE 3
Cloned Alu sequences that map to the regions
of putative linkage to major psychosis

| | Homology | | Evidence for linkage to |
|---|---|---|---|
| | length in bp; | Chr. | schizophrenia or bipolar |
| Clone Name | % Identity | Location | disorder (reference) |
| BD43- | 191 bp; 99.5% | 1p31 | Rice et al 1997 |
| RevE77m | | | |
| BD43 -A6m | 168 bp; 100% | 1q21 | Brzustowicz et al 2000 |
| BD43- | 192 bp; 100% | 5q22 | Straub et al 1997 |
| E78m | | | Camp et al 2001 |
| | | | Bennett et al 1997¹ |
| Sch56- | 189 bp; 99.5% | 6p22 | Kendler et al 2000 |
| E32m | | | Schwab et al 1995a |
| Sch37- | 144 bp; 99.4% | 10p15 | Straub et al 1998 |
| A9RR-m | 190 bp; 99.5% | 10p14 | DeLisi et al 2002 |
| Sch56- | 192 bp; 100% | | Faraone et al 1998 |
| E283m | | | Schwab et al 1998 |
| BD34- | | | |
| D19M | | | |
| BD34- | | | |
| E62m | | | |
| Sch56 -r- | 186 bp; 96.5% | 11q14 | Evans et al 1995; |
| 37m | | | Petit et al 1999² |
| BD43 -15m | 190 bp; 99.5% | 21q21 | Detera-Wadleigh et al |
| | | | 1996 |
| Sch74- | 206 bp; 97.7% | 22q12.2 | Pulver et al 1994 |
| E318_m | 193 bp; 100% | | Gill et al 1996 |
| Ctrl57-E4m | | | Kelsoe et al 2001; |
| | | | Myles-Worsley et al |
| | | | 1999 Schizophrenia |
| | | | Collabporative |
| | | | Linkage Group |
| | | | Mujaheed et al 2000 |
| | | | DeLisi et al 2002; |
| | | | Moises et al 1995 |
| | | | Schwab et al 1995b |
| 45 clones | 191 bp; 100% | Yq11.2 | Alitalo et al 1988³ |
| from affecteds | | Yq12 | Mors et al 2001⁴ |
| and 12 clones | | | |
| from controls | | | |
| Ctrl57-E6m | 187 bp; 99% | 1q31.1 | Detera-Wadleigh et al |
| Ctrl50- | 179 bp; 95% | | 1999 |
| RevE169m | | | |
| Ctrl57-E3m | 191 bp; 100% | 5q34 | Crowe and Vieland 1999 |
| Ctrl50- | 181 bp; 100% | 18q23 | Van Broeckhoven and |
| | | | Verheyen 1999; |
| E166m | | | Verheyen et al 1999 |
| | | | Ewald et al 1999 |
| | | | Freimer et al 1996 |

¹Interstitial deletion at 5q21-23.1 in an adult female with schizophrenia, mental retardation, and dysmorphic features.
²Schizophrenia-associated t(1; 11)(q42.1; q14.3) breakpoint region.
³Translocation with the breakpoints between Yq11.23 and Yq12, and in 15p11, respectively, in two brothers who both had schizophrenia.
⁴The occurrence of the combined phenotype including both schizophrenia and bipolar disorder was significantly increased among individuals with the 47, XYY karyotype.

References of only positive findings of linkage to major psychosis are listed in the table.

Several of the genes listed within Table 2 are of significant interest, for example, the gene for spinocerebellar ataxia type 1 (SCA1)(6p22) (Tab. 2). SCA1 contains a potentially unstable (CAG)n/(CTG)n trinucleotide repeat tract, which, when increased beyond the normal size, exhibits neurotoxic effects. In addition, the unstable trinucleotide repeats represent the molecular substrate for genetic anticipation, which, according to some authors (reviewed in (McInnis et al 1999)), is observed in major psychosis. Some case-control and family-based association studies revealed statistically significant evidence that this gene is a predisposing factor to SCH (Joo et al 1999; Wang et al 1996).

Other genes listed in Table 2, although less known in the field of psychiatric research, are also of significant interest. The embryonic ectoderm development gene (EED) (11q14) is necessary during gastrulation and organogenesis (Morin-Kensicki et al 2001). EED interacts with histone deacetylase (HDAC), a key player in the epigenetic regulation of chromatin structure, and the HDAC inhibitor trichostatin A, which relieves transcriptional repression mediated by EED (van der Vlag and Otte 1999). Another link to the regulation of gene transcription can be found in a transcriptional repressor GCF2 (2q37), which exhibits differential affinity-depending on the DNA methylation status in that DNA methylation at the binding site abrogates both protein binding and repressor activity (Eden et al 2001).

The gene encoding leukemia inhibitory factor (LIF) (22q12) is expressed in the brain (Lemke et al 1997), promotes cholinergic expression in several neuronal populations (Cheema et al 1998), and plays a role in neuronal development, determination of phenotype, survival, and response to nerve injury (Moon et al 2002). Densin-180 (1p31) is highly concentrated at synapses along dendrites and it has been suggested that this protein participates in specific adhesion between presynaptic and postsynaptic membranes at glutamatergic synapses. The mRNA encoding densin-180 is brain specific and is more abundant in forebrain than in cerebellum (Apperson et al 1996; Kennedy 1997). Four putative splice variants (A-D) of the cytosolic tail of densin-180 were shown to be differentially expressed during brain development (Strack et al 2000). In this connection, it is interesting to note that one of the hypomethylated Alu sequences was found in the vicinity of the gene encoding splicing factor 3A (22q12) that is essential for the formation of the mature 17S U2 snRNP and the prespliceosome (Nesic and Kramer 2001). Alternative RNA splicing is operating in a highly cell- and tissue-specific or developmentally specific manner. This directly applies to the neurons, where the functions of many gene products are regulated by alternative splicing (Shinozalci et al 1999). Differential splicing (e.g. mRNA for N-methyl-D-aspartate receptor (Le Corre et al 2000); dopamine D3 receptor (Karpa et al 2000)) has been implicated in SCH.

Several identified genes point at the putative immune and inflammatory components of major psychosis. Oncostatin M (OSM)(22q12) is a member of the interleukin (IL)-6 cytokine family that regulates inflammatory processes in the brain (Ruprecht et al 2001). Aiolos (17q12) encodes a hemopoietic-specific zinc finger transcription factor that is an important regulator of lymphocyte differentiation and is involved in the control of gene expression and, associated to nuclear complexes, participates in nucleosome remodeling (Schmitt et al 2002). It is not yet known if the gene encoding Aiolos can be expressed in the brain. A stress-responsive gene highly expressed in brain and reproductive organs (BRE) (2p23) is a house-keeping gene that may play a role in homeostasis or in certain pathways of differentiation in cells of neural, epithelial, and germ line origins (Li et al 1995). Over expression of BRE inhibited TNF-induced NF kappa B activation, indicating that the interaction of BRE protein with the cytoplasmic region of p55 TNF receptor may modulate signal transduction by TNF-alpha (Gu et al 1998).

Links to the metabolic stress in the affected brain is suggested by the gene encoding the AMP-activated protein kinase (beta 2 unit on chr 1q21). This kinase represents a heterotrimeric serine/threonine protein kinase with multiple isoforms for each subunit (alpha, beta, and gamma) and is activated under conditions of metabolic stress. It is widely expressed in many tissues, including the brain (Turnley et al 1999).

Epigenetic studies of retroelements can be a valuable analytical (and diagnostic) tool that complements the more traditional genetic linkage, association, and gene expression studies (Petronis et al 2000). Identification of the epigenetically dysregulated “junk” DNA sequences may allow for mapping of specific genomic regions in which genetic and/or epigenetic re-arrangements occurred. Such a retroelement may serve as a reporter, a signal that allows for the localization of genomic changes, and a mechanism for the dysfunction of genes that are localized in such regions and may be the actual cause of psychosis. Expression studies of the genes located in the vicinity of epigenetic reporters can provide further clues to the pathobiological pathways of a disease. Of particular interest may be mapping of differently regulated “junk” DNA elements performed in parallel with microarray-based global gene expression (Mirnics et al 2001). Large numbers of genes demonstrate differences in expression; however, it is never clear which changes are directly involved in the disease process and which ones just represent secondary ‘downstream’ changes and/or compensatory effects. There is no straightforward approach for how to separate the two groups of events in the affected cell, but the presence of epigenetic changes in only some of the differentially expressed genes and the absence of such changes in the others can provide clues for a cause-effect relationship in the myriad of molecular changes in the affected brain. Support for this idea comes from the array-based studies in breast cancer, which detected numerous differentially expressed genes in the malignant tissue and evident epigenetic deregulation of the otherwise impeccable BRCA1 (Hedenfalk et al 2001). Although the epigenetic status of other genes has not been investigated, hypermethylation of BRCA1 could certainly be one of the initiators of malignant growth.

Several Alu mapped loci have been of significant interest in linkage studies of major psychosis, including 1q21, 10p15, and 22q12, among numerous others (Table 3). Epigenetic mapping of hypomethylated retroelements may also facilitate genetic linkage studies. Traditional genetic linkage studies face major difficulties in fine mapping of the regions of susceptibility and identification of the actual gene dysfunction that leads to major psychosis. Typically, the regions that exhibit evidence for linkage to major psychosis are in the range of ˜10-15 mln nucleotides; furthermore, such regions may contain several hundred genes. Screening of such a large number of genes by traditional strategies for the detection of DNA variation is not a feasible task. Hypomethylated Alu's may pinpoint the very specific site of genomic DNA and the critical gene(s) epigenetic dysfunction that may have caused psychosis. It is necessary to note that the putative epigenetic dysfunction may exhibit stability during meiosis and therefore can be transmitted from one generation to another (Petronis 2001; Rakyan et al 2002), which would simulate familial cases of the disease.

### Example 2

### Identification of Strong Correlation Between Huntingdon's Disease and Hypomethylation in a Locus Having a Retroelement

Brain tissues. Samples from caudate and putamen (the brain regions that are primary sites of pathological changes in Huntington's disease [HD]) of HD patients (N=3; age at death 52+3 yr) and matched controls (n=4; age at death 54+3.5 yr) were analyzed.

Methods. Same as in Example 1 except for the following details. For the analysis of Alu sequences within the Huntington's disease (HD) gene, primers for two Alu sequences downstream of the (CAG)n/(CTG)n trinucleotide repeat region were synthesized. It is of note that in the HD locus analysis, concrete Alu sequences were investigated, and the designed primers were complementary to the flanking regions of each specific Alu of the HD gene. This approach tested if DNA modification is different in the regions surrounding Alu's within the gene that is known to cause a neuropsychiatric disease. The set of primers that amplified Alu located ˜4 Kb downstream of the (CAG)n/(CTG)n repeat region (NCBI ID: Z68756; Alu repeat region position 18,160 bp-18,448 bp) generated a visible PCR signal in the test experiments using genomic DNA as a template. This Alu was selected for further analysis in the HD patients and controls. PCR conditions for amplification of this fragment were as follows: 1× standard PCR buffer, containing dimethylsulphoxide (DMSO) 10%; 2.5 mM MgCl₂; 0.16 mM DNTP and 10 microMolar of each of HD primer (1MF: CAGCGTACACATACACAGAAGAGA (SEQ ID NO:4) and 1MR: TTCCTAGTCACCAAGTCATAGCA (SEQ ID NO:5)), and 1U of Taq: Pfu polymerases mix (9:1); 35 cycles at 94° C. for 30 sec, 55° C. for 30 sec, and 72° C. for 30 sec. PCR product size was ˜360 bp.

The Alu sequence located ˜4 Kb downstream of the (CAG)n/(CTG)n repeat region of the HD gene was exclusively amplified in the hypomethylated fraction of the striatum DNA extracted from all three HD patients, but from none of the hypomethylated fractions of the four controls. Thus, the striatum samples provided a 100% true positives and 0% false positives when diagnosing HD disease by identifying hypomethylation within a locus containing a retroelement. As such there is a strong correlation between HD disease and the identified locus.

The finding that HD Alu exhibited differential DNA methylation of the flanking regions in HD patients vs. controls supports the idea that epigenetic dysregulation of retroelements sequences can lead to disease, for example neuropsychiatric diseases. This finding, suggests that analysis of differentially modified retroelements and their flanking sequences can point at the etiological disease genes.

It is interesting to note that HD represents a classical genetic disorder caused by expansion of a (CAG)n/(CTG)n repeat tract. While epigenetic changes and their role in the disease have never been investigated in HD, there is indirect evidence that epigenetic factors may be operating in the regulation of the HD gene (Filippova et al 2001). The HD Alu data immediately linked to our finding of an Alu within the gene for spinocerebellar ataxia type 1 (SCA1)(6p22) (see Example 1; Table 2). Like HD, SCA1 contains a potentially unstable (CAG)n/(CTG)n trinucleotide repeat tract, which, when increased beyond the normal size, exhibits neurotoxic effects.

### Example 3

### Identification of Strong Correlation Between Huntingdon's Disease and Hypomethylation in a Locus Having a Retroelement

The same experiment as in Example 2 was repeated with 10 HD patients and 10 control subjects (see Table 4). DNA was extracted from cerebellum and striatum samples for each HD patient and control subject.

TABLE 4
Data on Huntington Disease patients and control cases

| Brain # | Distribution Dx | Age | Sex | PMI |
|---|---|---|---|---|
| B3976 | H3 | 73 | M | 23.00 |
| B4094 | H3 | 72 | M | 12.75 |
| B4381 | H4 | 55 | F | 24.40 |
| B5119 | H3 | 68 | F | 17.00 |
| B5146 | H3 | 79 | F | 16.25 |
| B5177 | H3 | 49 | M | 25.25 |
| B5331 | Control | 74 | M | 22.50 |
| B5077 | Control | 67 | M | 18.50 |
| B3813 | Control | 58 | F | 20.00 |
| B5176 | Control | 65 | F | 24.25 |
| B5113 | Control | 74 | F | 12.17 |
| B5270 | Control | 52 | M | 22.56 |
| B4781 | H4 | 56 | F | 9.50 |
| B4826 | H4 | 49 | M | 16.60 |
| B4828 | H4 | 52 | M | 18.16 |
| B5034 | H4 | 54 | M | 20.08 |
| B4739 | Control | 50 | M | 26.50 |
| B4751 | Control | 54 | M | 24.20 |
| B4974 | Control | 58 | F | 14.30 |
| B5024 | Control | 56 | M | 21.33 |

Where H3 is the preterminal stage of HD
H4 is the terminal stage of HD
PMI is the postmortem interval (time between death and a brain tissue sampling)

The Alu sequence located ˜4 Kb downstream of the (CAG)n/(CTG)n repeat region of the HD gene was exclusively amplified in the hypomethylated fraction of the cerebellum DNA extracted from all 10 HD patients, but from none of the hypomethylated fractions of the 10 controls. Thus, the cerebellum samples provided a 100% correlation between HD disease and hypomethylation within a locus containing a retroelement.

With respect to striatum samples, the Alu sequence located ˜4 Kb downstream of the (CAG)n/(CTG)n repeat region of the HD gene was found to be amplified in the hypomethylated fraction of DNA from 8 out of 10 HD patients, and from only 1 out of 10 of the hypomethylated fractions of the four controls.

These results corroborate the findings and conclusions of Example 2. Persons skilled in the art will recognize that the methods provided in Examples 2 and 3 can be used for diagnosis of Huntingdon's disease, including pre-diagnosis of Huntingdon's disease.

### Example 4

### Detection of Epigenetic Abnormalities Associated with Schizophrenia or Bipolar Disorder

Identification of the actual genes, which are epigenetically dysregulated and increase the risk to major psychosis, is not a simple task. Potentially any of the 35,000 human genes can be an epigenetic candidate for schizophrenia and bipolar disorder. The present invention provides for epigenetic analysis of multicopy DNA sequences leading to the identification of DNA sequences that predispose to major psychosis. At least 35% of the human genoome consists of numerous copies of different transposons dispersed in the genome (NB: only ˜5% of the human genome are exons, i.e. coding sequences of functional genes) (Yoder J A, Walsh C P, Bestor T H. Cytosine methylation and the ecology of intragenomic parasites. Trends Genetics, 13(8):335-40, 1997). The range of copies of repetitive DNA fragments varies widely: There are 10⁶ copies of Alu sequences and 10⁵ copies L1 elements per genome (ibid.). The general opinion is that such sequences represent excess baggage of our evolutionary heritage and do not perform any specific genomic function. This fraction of the genome is sometimes called “junk” or “parasitic” DNA. Such elements are not generally harmful to a cell as long as they do not exhibit any transcriptional activity and do not affect the integrity of the-host genome. Transcriptional inactivation of the multicopy elements is achieved by their epigenetic modification. It has been widely observed that DNA methylation plays a role in silencing various types of DNA sequences. Since it is becoming evident that DNA methylation may act in concert with histone acetylation (Nan X, Campoy F J, Bird A. MeCP2 is a transcriptional repressor with abundant binding sites in genomic chromatin. Cell, 88(4):471-81, 1997), chromatin conformation can also be considered a factor that plays a role in the inactivation of retrotransposons as well as any other newly integrated DNA sequence. The findings that Alu and L1 elements as well as numerous other retroelements are methylated and transcriptionally inactive in the genomes of fungi, plants, and mammals provided the basis for postulating that epigenetic DNA modification represents a host genome defense system (Bestor T H. DNA methyltransferase in genome defence. In: Epigenetic mechanisms of gene regulation. Eds: Russo V E A, Martienssen R A, Riggs A D. Cold Spring Harbor Laboratory Press, pp. 61-76, 1996; Yoder J A, Walsh C P, Bestor T H. Cytosine methylation and the ecology of intragenomic parasites. Trends Genetics, 13(8):335-40, 1997).

The epigenetic parameter may add a new dimension to the already available developments in psychiatric research. In our experiments we serendipitously detected that while the overwhelming majority of Alu sequences in the genomic DNA extracted from human brain are methylated, a small fraction of such sequences is unmethylated. The origin of such selective Alu demethylation is not clear. Without wishing to be bound by theory, this most likely represents a local failure of the epigenetic host defense system, which has no direct impact to the normal functioning of the brain. On the other hand, such local epigenetic changes may not be limited to the Alu sequences and may extend to the surrounding genes, causing dysregulation which may be detrimental to the cells. Supporting evidence for this comes from the observation that retroelements may become demethylated because they are located in the genomic region that was subjected to genetic and epigenetic re-organization. In malignant cells, it was detected that some Alu (Rubin C M, VandeVoort C A, Teplitz R L, Schmid C W. Alu repeated DNAs are differentially methylated in primate germ cells. Nucleic Acids Research, 22(23):5121-7, 1994; Sinnett D, Richer C, Deragon J M, Labuda D. Alu RNA transcripts in human embryonal carcinoma cells. Model of post-transcriptional selection of master sequences. Journal of Molecular Biology, 226(3):689-706, 1992) and L1 (FlorI A R, Franke K H, Niederacher D, Gerharz C D, Seifert H H, Schulz W A. DNA methylation and the mechanisms of CDKN2A inactivation in transitional cell carcinoma of the urinary bladder. Laboratory Investigation, 80(10):1513-22, 2000; Jurgens. B, Schmitz-Drager B J, Schulz W A. Hypomethylation of L1 LINE sequences prevailing in human urothelial carcinoma. Cancer Research, 56(24):5698-703, 1996) elements became hypomethylated and transcriptionally active.

The present invention provides for identification of unmethylated “junk” DNA sequences in major psychosis allowing for mapping of specific genomic regions in which epigenetic re-arrangements occurred. Dysfunction of genes that are localized such regions may be the actual cause of psychotic symptoms, while the demethylated multicopy element sequence would serve as a reporter, a signal that allows for localization of epigenetic changes in the genome.

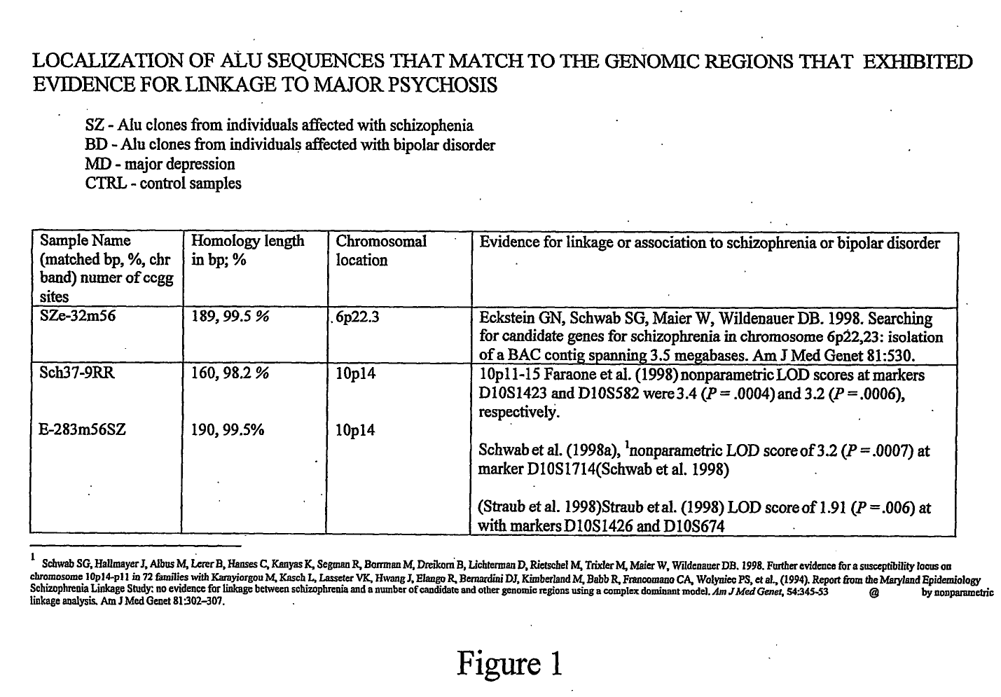

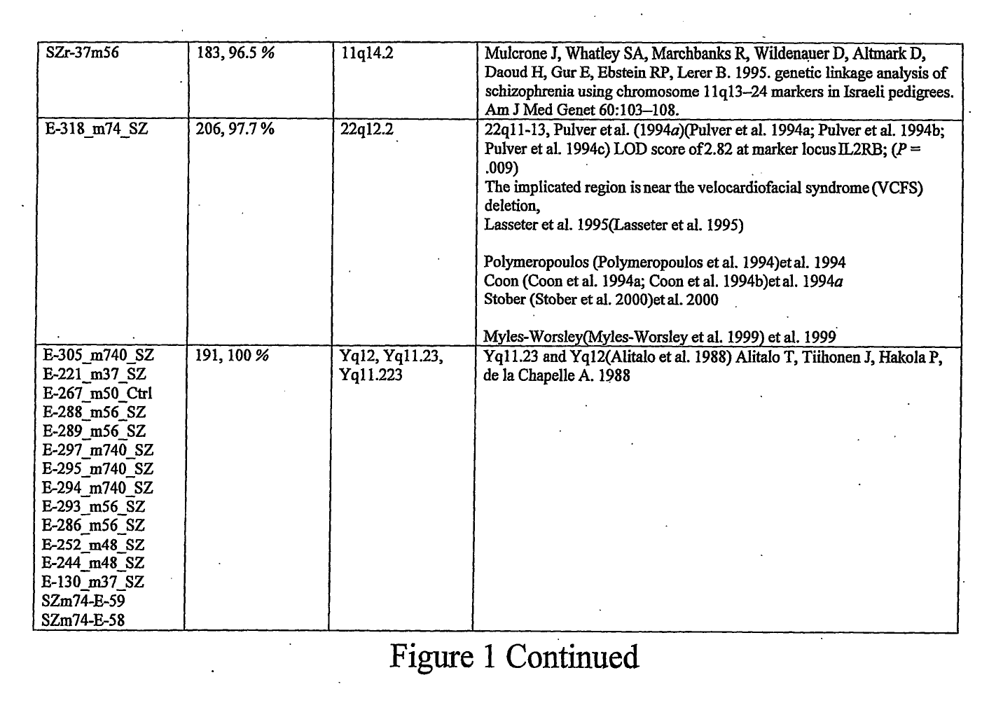

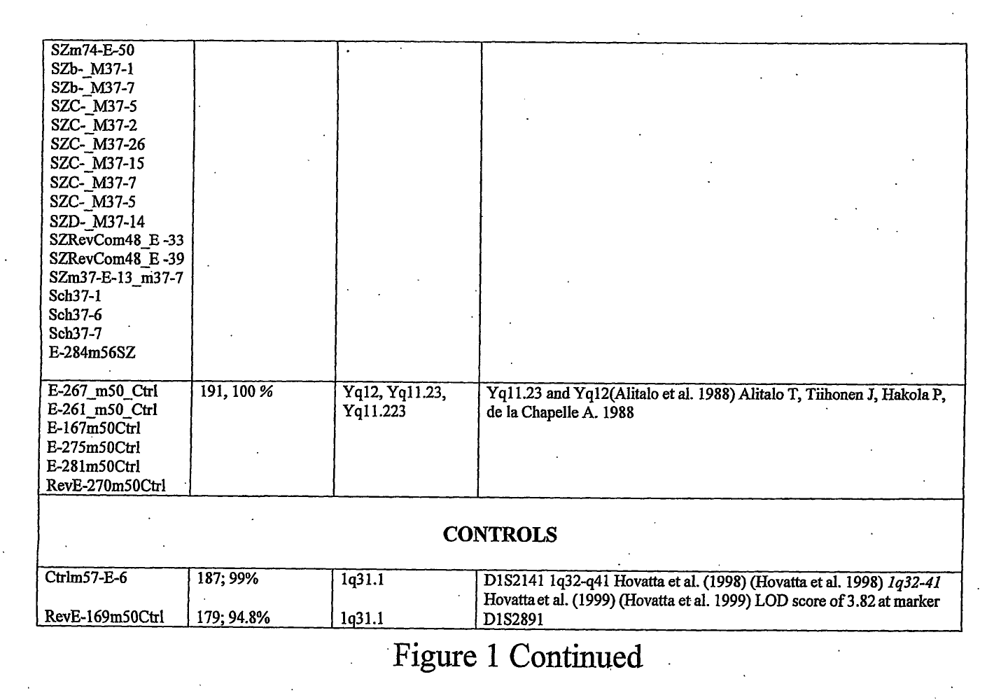

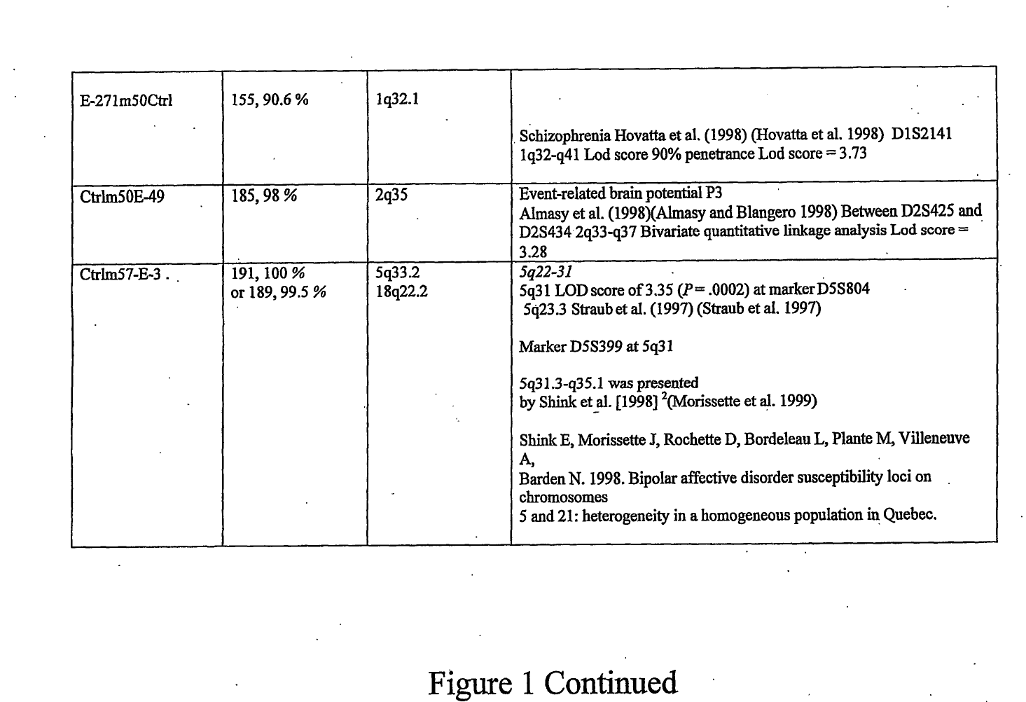

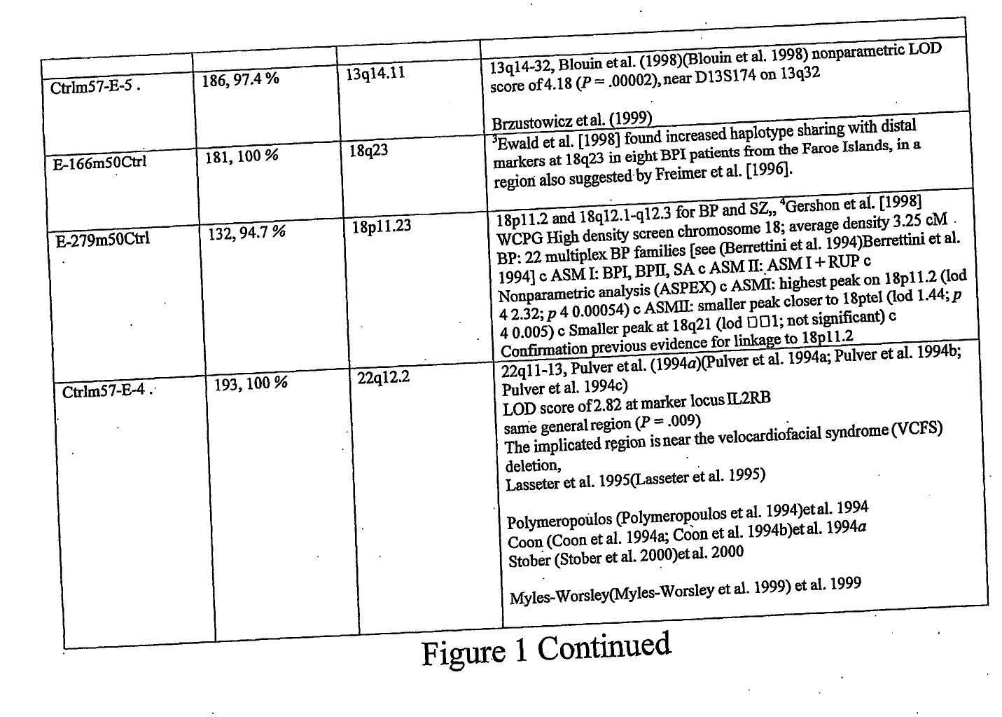

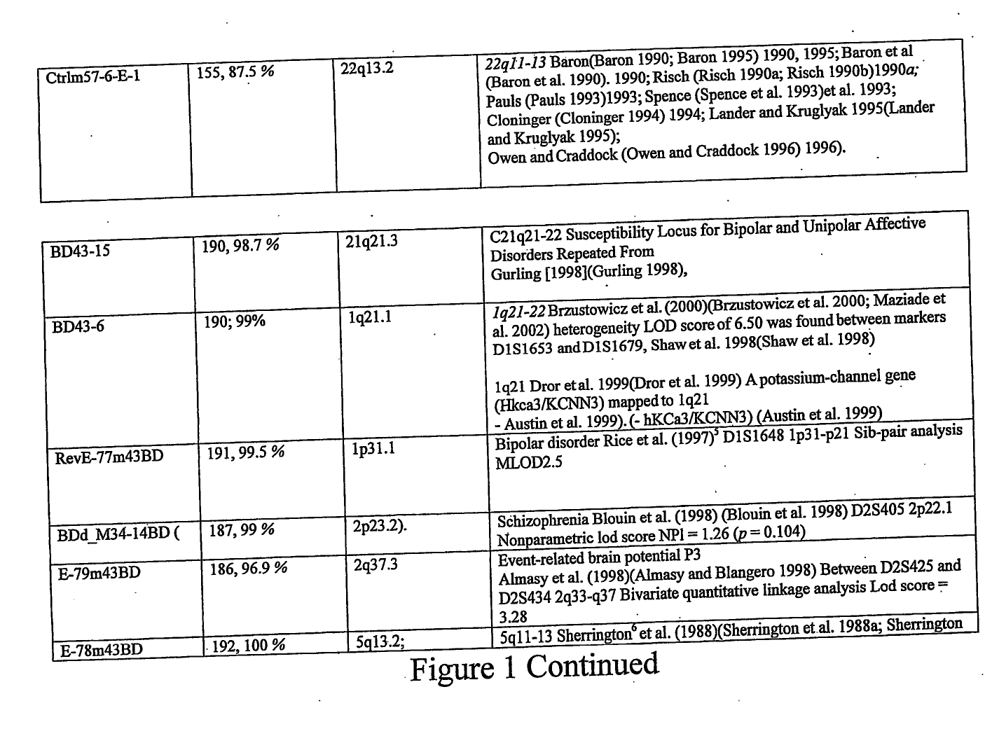

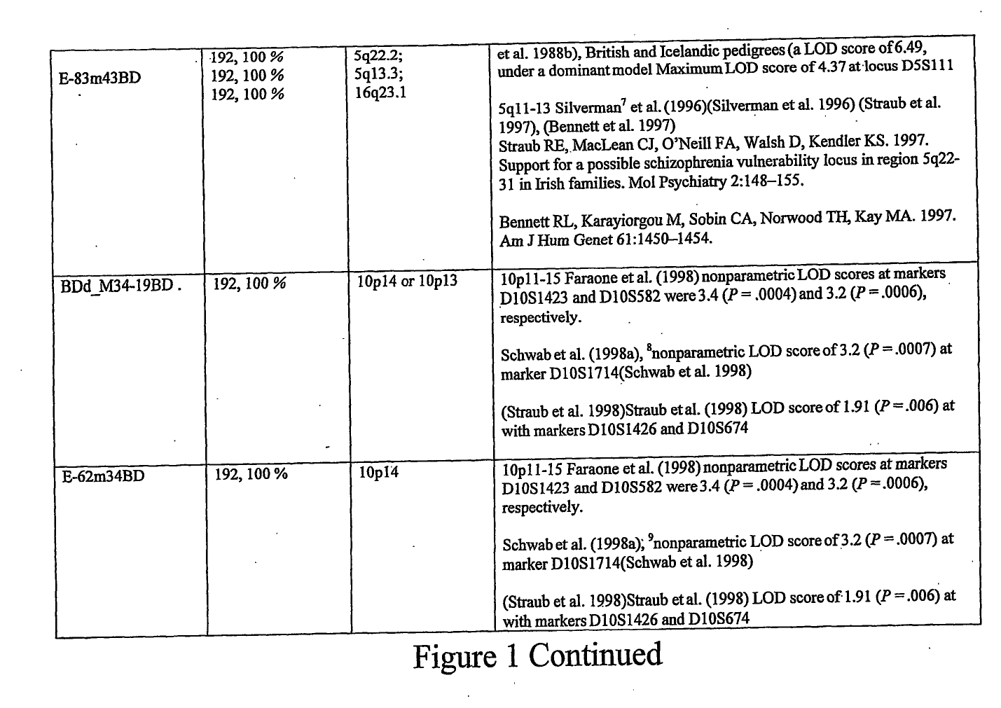

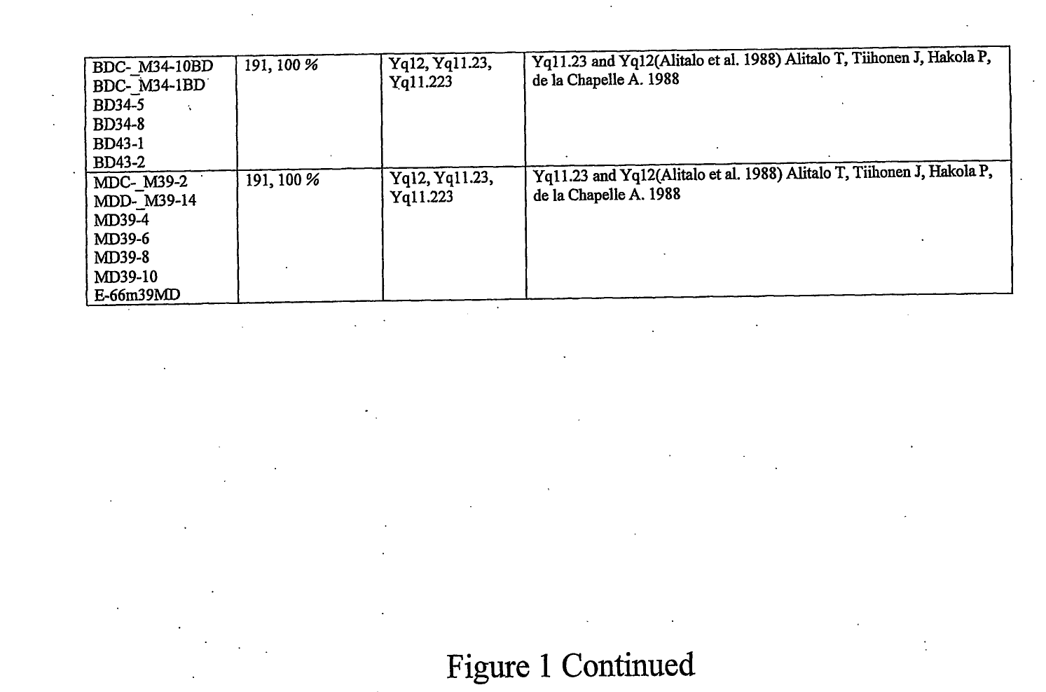

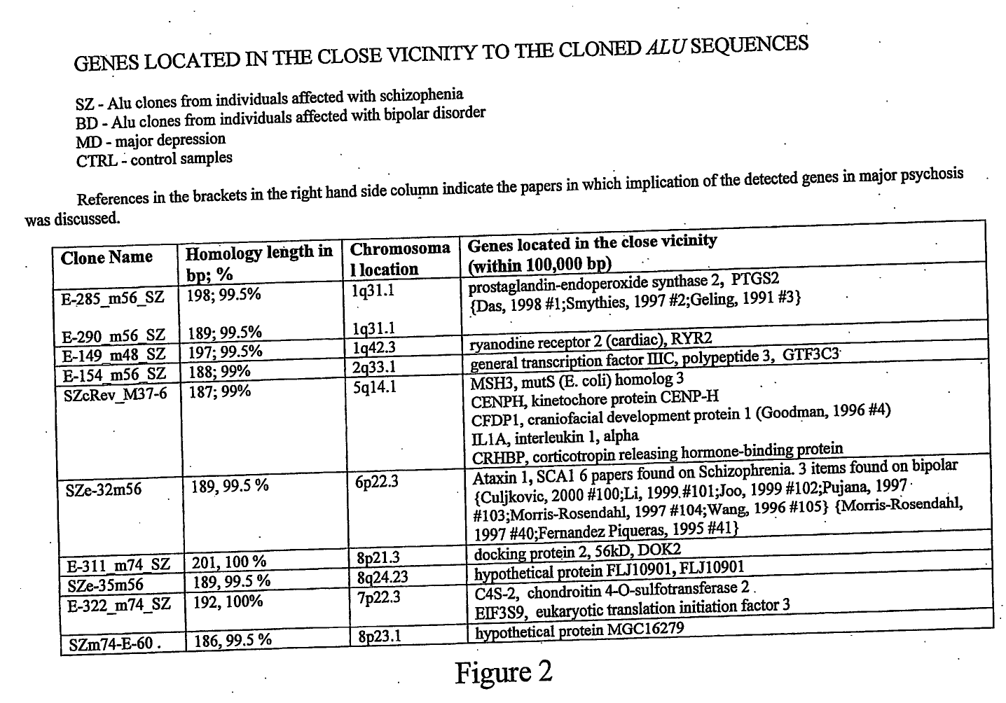

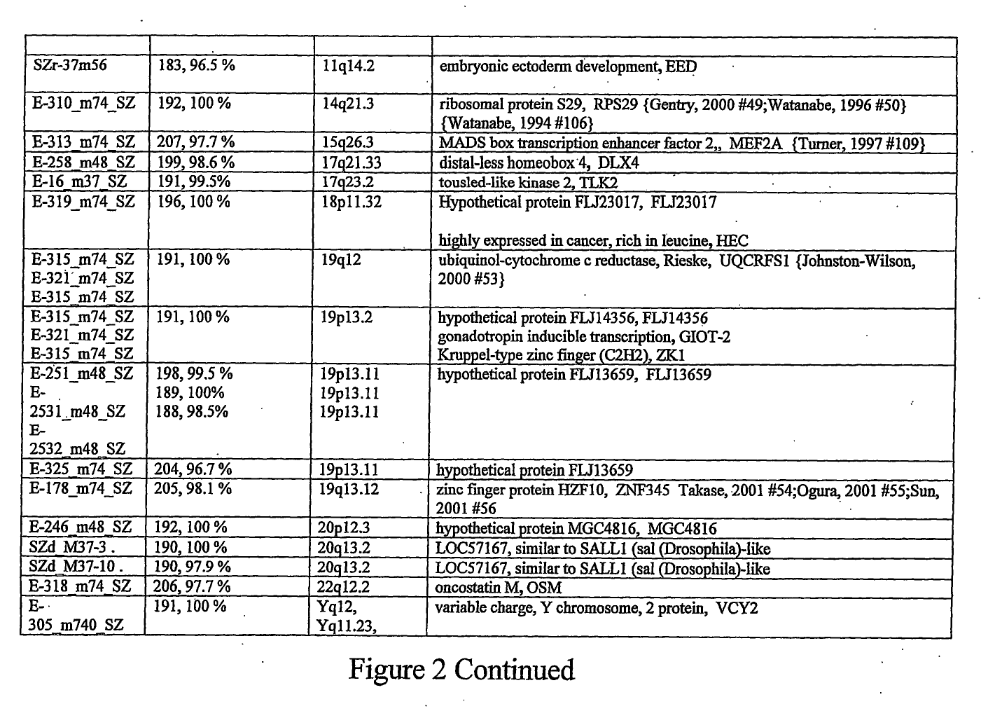

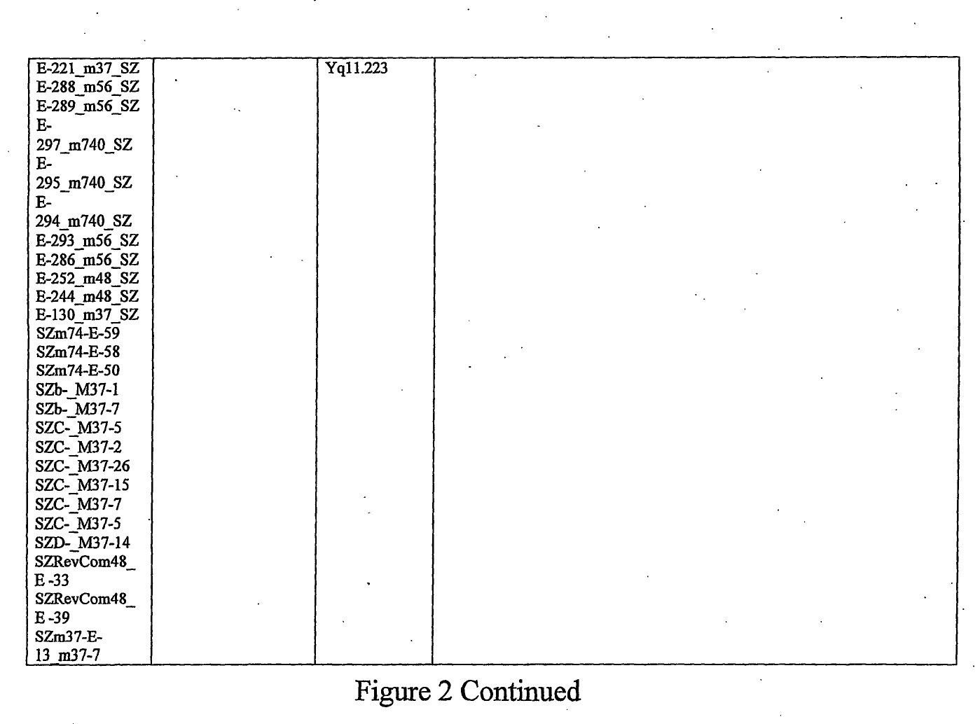

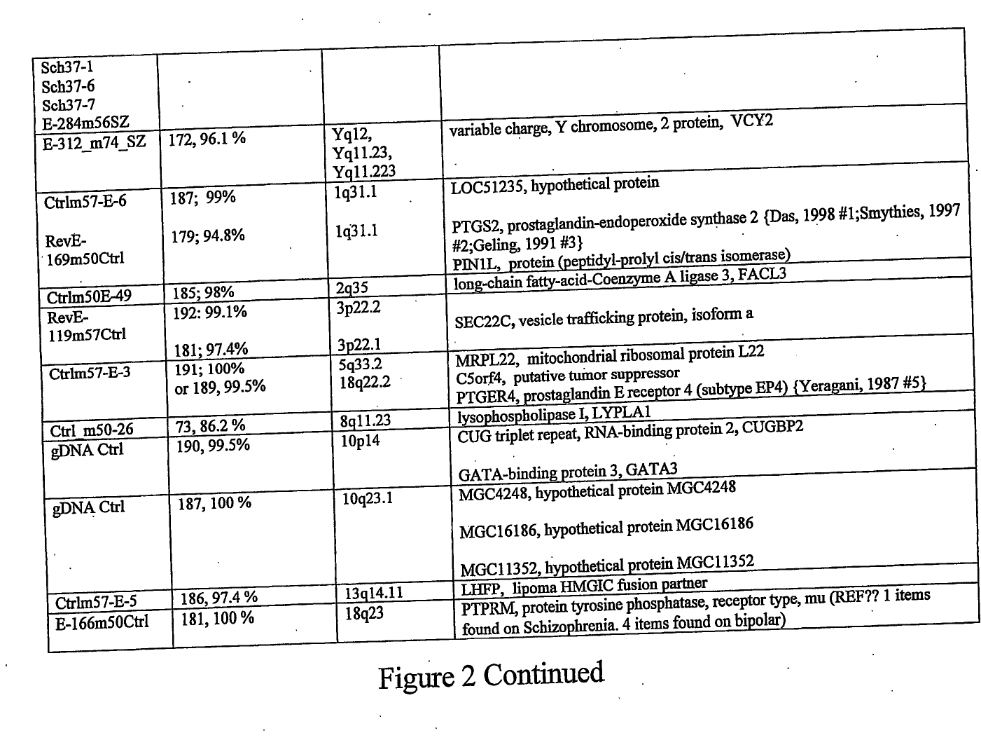

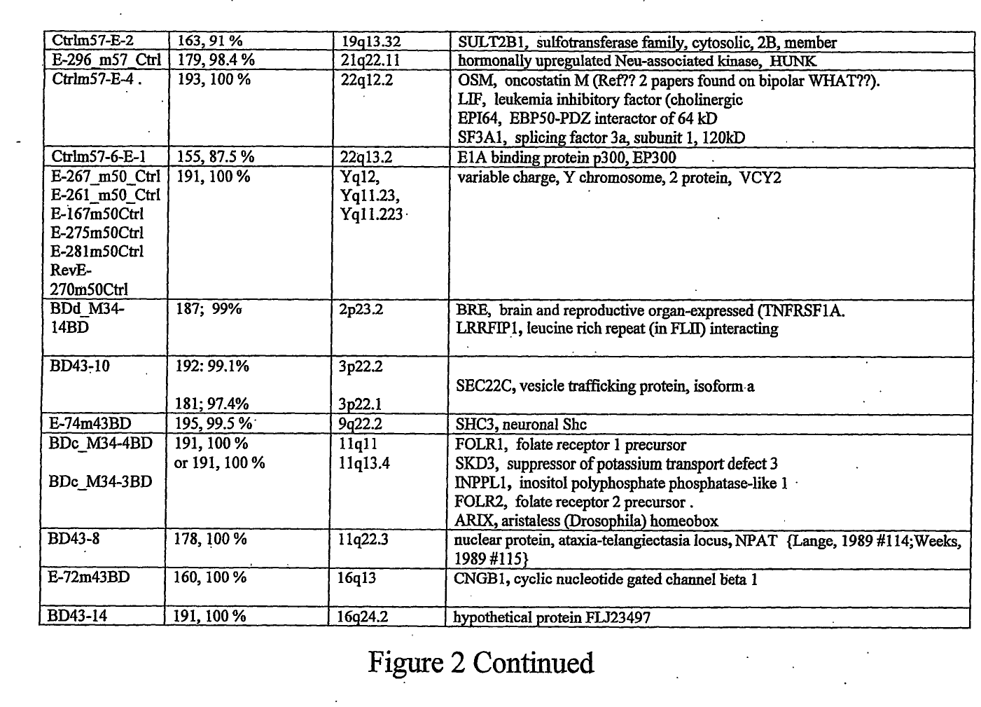

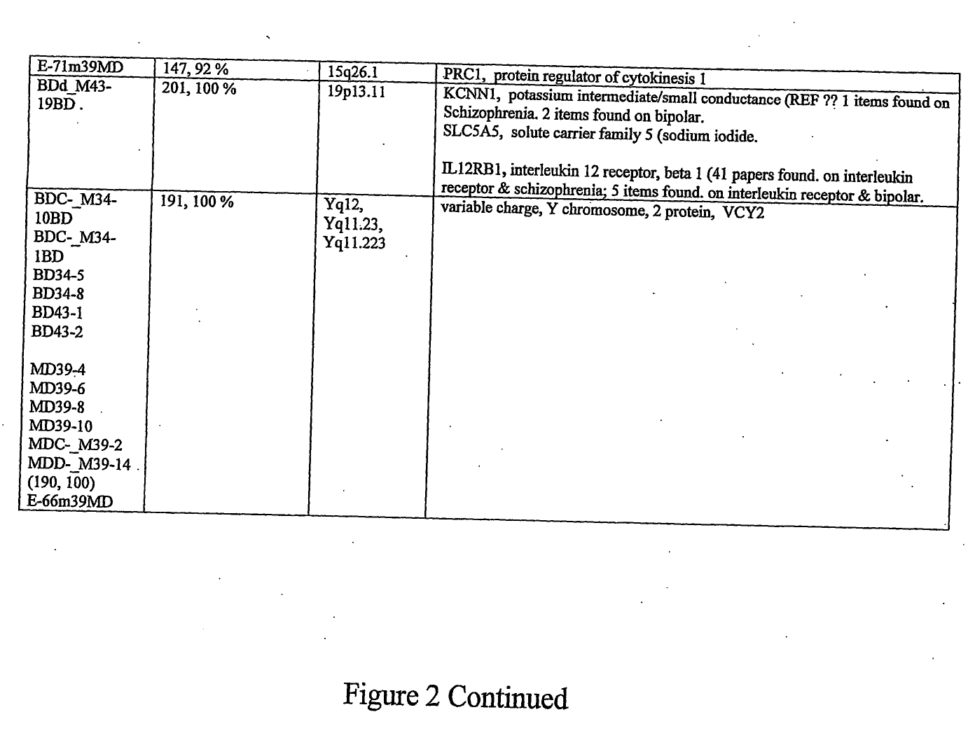

DNA samples were extracted from the frontal cortex of 40 post-mortem brain tissues of individuals who were affected with schizophrenia and bipolar disorder as well as control individuals. In order to avoid artifacts related to partial brain DNA degradation (which may simulate hypomethylation and produce artifactual Alu amplification; see below), the following procedure was performed. Undigested total genomic DNA was fractionated on an agarose gel, the high molecular weight (>15-20 kb) DNA was cut from the gel. The gel block, containing DNA, was treated with a gel digesting enzyme, agarase. Without any additional procedures, such high quality DNA samples can be further digested with a specific restriction enzyme and subjected to further analyses. The methylation sensitive restriction enzyme, HpaII, was used for digestion of DNA and the unmethylated fraction of brain specific DNA (fragments smaller than arbitrarily selected 61 kb) were separated from the methylated fraction of DNA using gel electrophoresis. The <6 kb fragments were purified from the gel using glass mill. Screening for the presence of Alu's in the purified unmethylated DNA was performed using PCR and primers complementary to the Alu sequence. Alu amplicons were cloned into a vector and transformed into E. coli XL1-blue. Up to ten recombinant clones from each PCR product were sequenced from six individuals affected with major psychosis and four controls. The location of such Alu sequences were identified using human genome databases (http://genome.ucsc.edu/). It was detected that the Alu's from affected individuals in numerous cases corresponded with the genomic regions that showed evidence for linkage in genetic linkage studies of major psychosis. For example, one of the Alu sequences cloned from an affected individual mapped to chr 1q21, the region that was linked to schizophrenia (lod score of 6.5, the strongest evidence for linkage in schizophrenia genetics thus far) in large multiplex schizophrenia families (Brzustowicz L M, et al., 2000). In addition, an Alu clone from another psychosis patient exhibited sequence homology with 1q42, the translocation region in a schizophrenia kindred (St Clair D, et al. 1990). Other genomic regions where Alu sequences mapped to the linkage ‘spots’, include 5q11 (although linkage to this region [Sherrington R, et al.1988] was not replicated in other studies, two large kindreds exhibit lod scores between 2 and 3 in favor of linkage). Other identified regions include: 5q35 (chr 5 data reviewed in Crowe R R, et al. 1999), 8p23 (lod score 3.8 in a large Swedish schizophrenia kindred), 8p21, 10p14, the pericentrometric regions of chr 10 and 10q26 (Wildenauer D B, et. al. 1999), 11p15 and 11q13, 14q32 (Craddock 1999), 12p13 and 12q23-24 (Detera-Wadleigh S D. et al. 1999), and 22q13 (Nurnberger J I Jr, et al.1999). The 22q13 region exhibited evidence for linkage in numerous studies and harbors a deletion region in velo-cardiofacial syndrome, a disorder quite often resulting in psychotic symptoms (Chow E W, et al. 1994). For more details on the localization of the cloned Alu sequences see FIG. 1. Alu sequences that are located in the vicinity (within 100,000 bp) of coding genes are listed in FIG. 2. Sequences of the cloned Alu's are provided in FIG. 3.

The above results are of interest for the following reasons. First, clustering of the Alu sequences into the groups of affected individuals and controls, if replicated in an independent sample, would indicate that epigenetic changes of repetitive DNA elements in some genomic loci are specific to major psychosis. This would be a significant step forward in the light of the myriad of non-specific molecular changes in the brains of patients affected with major psychosis. Second, genomic location of the hypomethylated Alu's match with the loci that exhibit evidence for linkage to major psychosis. Traditional genetic linkage studies face major difficulties in fine mapping of the regions of susceptibility and identification of the actual gene dysfunction that leads to major psychosis. Typically the regions that exhibit evidence for linkage to major psychosis are in the range of ˜10-40 cM, i.e. ˜10-40 million nucleotides (Thaker G K, et al., 2001; Tsuang M T, et al. 2001; Bray N J, and Owen M J. 2001: Gershon E S. 2000; Nurnberger J I Jr, et al. 2000), and such regions contain hundreds of genes. Screening of such a large number of genes by traditional strategies for the detection of DNA variation is not possible. For fine mapping of prediposing genes using the transmission disequilibrium test, very large samples are required; this strategy has not been productive in psychiatric research thus far. In conclusion, the “junk” DNA-based search for major psychosis genes may represent a valuable ‘shortcut’ in the identification of such genes. Hypomethylated Alu's may pinpoint very specific sites of genomic DNA epigenetic dysfunction of which may cause major psychosis.

### Example 5

### Identification of Genes Involved in Etiology of Schizophrenia or Bipolar Disorder Based on Epigenetic Analysis

The genes that are located in the regions exhibiting both linkage to major psychosis and epigenetic abnormalities in Alu sequences are subjected to a detailed analysis. Using the Celera Human Genome Database a list of genes from 1q21, 5q11, 8p23, 10p14, 11p15, 12p13, 12q23-24, 22q13, chr Y, and several other loci are selected for further investigation from the epigenetic point of view. The list includes ˜30 genes. Patients and controls are matched for age, sex, and race. Cases with drug and alcohol abuse are not used in the study. Treatment with neuroleptic medications is also a significant confounding factor. Neuroleptic naive schizophrenic patients are very rare, but cases with long neuroleptic free pre-mortem intervals are quite common. For example, in a recent study, one third of brain samples were neuroleptic-free for more than 6 months (Hernandez I, et al., 2000) and during this period, ˜50% of schizophrenia patients are expected to relapse (Viguera A C, et al., 1997). Epigenetic dysregulation in schizophrenia and bipolar disorder, and other disease associated epigenetic abnormalities in the brain may recur after neuroleptic treatment is stopped. Regarding the sample size, since there are no precedents of epigenetic studies in major psychosis, power analysis on the sample size is not possible. The investigation has been initiated with a relatively large sample by post-mortem brain study standards.

The prefrontal cortex from 25 post-mortem patients affected with major psychosis with >6 months of neuroleptic free period before death and a similar number of controls are used in the investigation. Over 70 brain samples from individuals who were affected with schizophrenia or bipolar disorder as well as controls are available at our laboratory and this sample increases every year. Total mRNA from the brain tissues is extracted using standard RNA extraction techniques (Chomczynski P,et al., 1987) and subjected to reverse transcription and quantitative PCR amplification using the Bio-Rad Real Time PCR equipment (http://www.bio-rad.com/iCycler/). This experiment allows for the quantitative evaluation of the steady state level of the candidate gene. ‘Is it β-actin’ mRNA serves as an internal standard for the degree of mRNA degradation. Expression of Is it β-actin is independent of the age of an individual and treatment (Schramm M, et al., 1999) and therefore can be reliably used as an estimate of the degree of post-mortem degradation. Steady state mRNA level of each individual gene is normalised according to its Is it β-actin mRNA data. The null hypothesis is that the group of affected individuals exhibits no differences in the steady state mRNA levels of the selected genes in comparison to the group of controls. The genes that reject the null hypothesis, i.e. the ones that exhibit statistically significant differences in steady state mRNA levels in affected tissues versus controls, are subjected to further analysis. The problem is that not all genes that exhibit significant differences in expression may carry epigenetic defects. Cases when changes in steady state mRNA levels that may occur within hours or even minutes after some triggers are applied, in the absence in any epigenetic changes in the genome have to be excluded. Typically, epigenetic DNA modification targets cytosines in CpG dinucleotides, each of which can be either methylated (metC) or unmethylated (C). The gold standard technique for DNA methylation analysis is based on the reaction of genomic DNA with sodium bisulfite under conditions such that cytosine is deaminated to uracil but metC remains unreacted (Frommer M, et al. 1992). Sequencing of bisulfite modified DNA reveals which cytosines were methylated and which cytosines were not. This approach has been fully operationalized in our laboratory (Popendikyte V, et al., 1999). The present invention provides for identifying one or more than one DNA coding sequences, from the list of ˜30 candidates, exhibiting disease specific epigenetic abnormality.

All references are herein incorporated by reference.

### REFERENCES

Alitalo T, Tiihonen J, Hakola P, de la Chapelle A (1988): Molecular characterization of a Y;15 translocation segregating in a family. Hum Genet 79:29-35.

Allen N D, Norris M L, Surani M A. Epigenetic control of transgene expression and imprinting by genotype-specific modifiers. Cell Jun. 1, 1990;61(5):853-61

Apperson M L, Moon I S, Kennedy M B (1996): Characterization of densin-180, a new brain-specific synaptic protein of the O-sialoglycoprotein family. J Neurosci 16:6839-52.

Bassett A S, Chow E W, Waterworth D M, Brzustowicz L (2001): Genetic insights into schizophrenia. Can J Psychiatry 46:131-7.

Bennett R L, Karayiorgou M, Sobin C A, Norwood T H, Kay M A (1997): Identification of an interstitial deletion in an adult female with schizophrenia, mental retardation, and dysmorphic features: further support for a putative schizophrenia-susceptibility locus at 5q21-23.1. Am J Hum Genet 61:1450-4.

Berrettini W (2002): Review of bipolar molecular linkage and association studies. Curr Psychiatry Rep 4:124-9.

Berrettini W H (2000a): Are schizophrenic and bipolar disorders related? A review of family and molecular studies. Biol Psychiatry 48:531-8.

Berrettini W H (2000b): Susceptibility loci for bipolar disorder: overlap with inherited vulnerability to schizophrenia. Biol Psychiatry 47:245-51.

Bestor T H. DNA methyltransferase in genome defence. In: Epigenetic mechanisms of gene regulation. Eds: Russo V E A, Martienssen R A, Riggs A D. Cold Spring Harbor Laboratory Press, pp. 61-76, 1996.

Bray N J, Owen M J. Searching for schizophrenia genes. Trends Mol Med. 2001; 7(4):169-74.

Brzustowicz L M, Hodgkinson K A, Chow E W, Honer W G, Bassett A S. Location of a major susceptibility locus for familial schizophrenia on chromosome 1q21-q22.Science Apr. 28, 2000;288(5466):678-82

Camp N J, Neuhausen S L, Tiobech J, Polloi A, Coon H, Myles-Worsley M (2001): Genomewide multipoint linkage analysis of seven extended Palauan pedigrees with schizophrenia, by a Markov-chain Monte Carlo method. Am J Hum Genet 69:1278-89.

Cardno A G, Rijsdijk F V, Sham P C, Murray R M, McGuffin P (2002): A twin study of genetic relationships between psychotic symptoms. Am J Psychiatry 159:539-45.

Cavalli G, Paro R. The Drosophila Fab-7 chromosomal element conveys epigenetic inheritance during mitosis and meiosis. Cell 1998; 93(4):505-18

Cheema S S, Arumugam D, Murray S S, Bartlett P F (1998): Leukemia inhibitory factor maintains choline acetyltransferase expression in vivo. Neuroreport 9:363-6.

Chomczynski P, Sacchi N. Single-step method of RNA isolation by acid guanidinium thiocyanate-phenol-chloroform extraction. Anal Biochem. April 1987; 162(1):156-9.

Chow E W, Bassett A S, Weksberg R. Velo-cardio-facial syndrome and psychotic disorders: implications for psychiatric genetics. Am J Med Genet 1994;54(2): 107-12

Craddock N, Lendon C. Chromosome Workshop: chromosomes 11, 14, and 15. Am J Med Genet. Jun. 18, 1999;88(3):244-54.

Crowe R R, Vieland V. Report of the Chromosome 5 Workshop of the Sixth World Congress on Psychiatric Genetics. Am J Med Genet. Jun. 18, 1999; 88(3):229-32.

DeLisi L E, Shaw S H, Crow T J, et al (2002): A genome-wide scan for linkage to chromosomal regions in 382 sibling pairs with schizophrenia or schizoaffective disorder. Am J Psychiatry 159:803-12.

Detera-Wadleigh S D. Chromosomes 12 and 16 workshop. Am J Med Genet. Jun. 18, 1999; 88(3):255-9.

Detera-Wadleigh S D, Badner J A, Berrettini W H, et al (1999): A high-density genome scan detects evidence for a bipolar-disorder susceptibility locus on 13q32 and other potential loci on 1q32 and 18p11.2. Proc Natl Acad Sci USA 96:5604-9.

Detera-Wadleigh S D, Badner J A, Goldin L R, et al (1996): Affected-sib-pair analyses reveal support of prior evidence for a susceptibility locus for bipolar disorder, on 21q. Am J Hum Genet 58:1279-85.

Eden S, Constancia M, Hashimshony T, et al (2001): An upstream repressor element plays a role in Igf2 imprinting. Embo J 20:3518-25.

Ehrlich M and Ehrlich K (1993) Effect of DNA methylation and the binding of vertebrate and plant proteins to DNA. In: Jost J P and Saluz P (eds) DNA Methylation: Molecular Biology and Biological Significance pp. 145-168. Birkhauser Verlag, Basel, Switzerland.

Evans K L, Brown J, Shibasaki Y, et al (1995): A contiguous clone map over 3 Mb on the long arm of chromosome 11 across a balanced translocation associated with schizophrenia. Genomics 28:420-8.

Ewald H, Wang A G, Vang M, Mors O, Nyegaard M, Kruse T A (1999): A haplotype-based study of lithium responding patients with bipolar affective disorder on the Faroe Islands. Psychiatr Genet 9:23-34.

Faraone S V, Matise T, Svrakic D, et al (1998): Genome scan of European-American schizophrenia pedigrees: results of the NIMH Genetics Initiative and Millennium Consortium. Am J Med Genet 81:290-5.

Filippova G N, Thienes C P, Penn B H, et al (2001): CTCF-binding sites flank CTG/CAG repeats and form a methylation-sensitive insulator at the DM1 locus. Nat Genet 28:335-43.

Florl A R, Franke K H, Niederacher D, Gerharz C D, Seifert H H, Schulz W A. DNA methylation and the mechanisms of CDKN2A inactivation in transitional cell carcinoma of the urinary bladder. Laboratory Investigation, 80(10):1513-22, 2000.

Freimer N B, Reus V I, Escamilla M A, et al (1996): Genetic mapping using haplotype, association and linkage methods suggests a locus for severe bipolar disorder (BPI) at 18q22-q23. Nat Genet 12:436-41.

Frommer M, McDonald L E, Millar D S, Collis C M, Watt F, Grigg G W, Molloy P L, Paul C L. A genomic sequencing protocol that yields a positive display of 5-methylcytosine residues in individual DNA strands. Proc Natl Acad Sci USA 1992;89:1827-31.

Gershon E S. Bipolar illness and schizophrenia as oligogenic diseases: implications for the future. Biol Psychiatry. Feb. 1, 2000;47(3):240-4.

Gill M, Vallada H, Collier D, et al (1996): A combined analysis of D22S278 marker alleles in affected sib-pairs: support for a susceptibility locus for schizophrenia at chromosome 22q12. Schizophrenia Collaborative Linkage Group (Chromosome 22). Am J Med Genet 67:40-5.

Gonzalgo, M. L. and Jones, P. A. (1997) Mutagenic and epigenetic effects of DNA methylation. Mutat. Res. 386(2), 107-18

Gottesman I I. Schizophrenia Genesis: The Origins of Madness. New York: W.H. Freeman; 1991.

Gu C, Castellino A, Chan J Y, Chao M V (1998): BRE: a modulator of TNF-alpha action. Faseb J 12:1101-8.

Hedenfalk I, Duggan D, Chen Y, et al (2001): Gene-expression profiles in hereditary breast cancer. N Engl J. Med 344:539-48.

Henikoff S, Matzke M A Exploring and explaining epigenetic effects. Trends Genet 1997;13(8):293-5

Hernandez I, Sokolov B P. Abnormalities in 5-HT2A receptor mRNA expression in frontal cortex of chronic elderly schizophrenics with varying histories of neuroleptic treatment. J Neurosci Res. 2000; 59(2):218-25.

Johnston-Wilson N L, Sims C D, Hofmann J P, et al (2000): Disease-specific alterations in frontal cortex brain proteins in schizophrenia, bipolar disorder, and major depressive disorder. The Stanley Neuropathology Consortium. Mol Psychiatry 5:142-9.

Jones P L, Veenstra G J, Wade P A, Vermaak D, Kass S U, Landsberger N, Strouboulis J, and Wolffe A P (1998) Methylated DNA and MeCP2 recruit histone deacetylase to repress transcription. Nature Genetics 19: 187-91.

Joo E J, Lee J H, Cannon T D, Price R A (1999): Possible association between schizophrenia and a CAG repeat polymorphism in the spinocerebellar ataxia type 1 (SCA1) gene on human chromosome 6p23. Psychiatr Genet 9:7-11.

Jurgens B, Schmitz-Drager B J, Schulz W A. Hypomethylation of L1 LINE sequences prevailing in human urothelial carcinoma. Cancer Research, 56(24):5698-703, 1996.

Karlsson H, Bachmann S, Schroder J, McArthur J, Torrey E F, Yolken R H (2001): Retroviral RNA identified in the cerebrospinal fluids and brains of individuals with schizophrenia. Proc Natl Acad Sci USA 98:4634-9.

Karpa K D, Lin R, Kabbani N, Levenson R (2000): The dopamine D3 receptor interacts with itself and the truncated D3 splice variant d3nf: D3-D3nf interaction causes mislocalization of D3 receptors. Mol Pharmacol 58:677-83.

Kelsoe J R, Spence M A, Loetscher E, et al (2001): A genome survey indicates a possible susceptibility locus for bipolar disorder on chromosome 22. Proc Natl Acad Sci USA 98:585-90.

Kendler K S, Myers J M, O'Neill F A, et al (2000): Clinical features of schizophrenia and linkage to chromosomes 5q, 6p, 8p, and 10p in the Irish Study of High-Density Schizophrenia Families. Am J Psychiatry 157:402-8.

Kennedy M B (1997): The postsynaptic density at glutamatergic synapses. Trends Neurosci 20:264-8.

Klar A J. Propagating epigenetic states through meiosis: where Mendel's gene is more than a DNA moiety. Trends Genet 1998; 14(8):299-301.

Lander E, Kruglyak L (1995): Genetic dissection of complex traits: guidelines for interpreting and reporting linkage results. Nature Genetics 11:241-247.

Le Corre S, Harper C G, Lopez P, Ward P, Catts S (2000): Increased levels of expression of an NMDARI splice variant in the superior temporal gyrus in schizophrenia. Neuroreport 11:983-6.

Lemke R, Gadient R A, Patterson P H, Bigl V, Schliebs R (1997): Leukemia inhibitory factor (LIF) mRNA-expressing neuronal subpopulations in adult rat basal forebrain. Neurosci Lett 229:69-71.

Li L, Yoo H, Becker F F, Ali-Osman F, Chan J Y (1995): Identification of a brain- and reproductive-organs-specific gene responsive to DNA damage and retinoic acid. Biochem Biophys Res Commun 206:764-74.

Li T H, Kim C, Rubin C M, Schmid C W (2000): K562 cells implicate increased chromatin accessibility in Alu transcriptional activation. Nucleic Acids Res 28:3031-9.

Li T H, Schmid C W (2001): Differential stress induction of individual Alu loci: implications for transcription and retrotransposition. Gene 276:135-41.

Lyko, F. and Paro, R. (1999) Chromosomal elements conferring epigenetic inheritance. Bioessays 21(10), 824-32.

McInnis M G, McMahon F J, Crow T, Ross C A, DeLisi L E (1999): Anticipation in schizophrenia: a review and reconsideration. Am. J Med Genet 88:686-93.

McNeil T F (1995): Perinatal risk factors and schizophrenia: selective review and methodological concerns. Epidemiol Rev 17:107-12.

Miniou P, Bourc'his D, Molina Gomes D, Jeanpierre M, Viegas-Pequignot E (1997): Undermethylation of Alu sequences in ICF syndrome: molecular and in situ analysis. Cytogenet Cell Genet 77:308-13.

Mirnics K, Middleton F A, Lewis D A, Levitt P (2001): Analysis of complex brain disorders with gene expression microarrays: schizophrenia as a disease of the synapse. Trends Neurosci 24:479-86.

Moises H W, Yang L, Li T, et al (1995): Potential linkage disequilibrium between schizophrenia and locus D22S278 on the long arm of chromosome 22. Am J Med Genet 60:465-7.

Moon C, Yoo J Y, Matarazzo V, Sung Y K, Kim E J, Ronnett G V (2002): Leukemia inhibitory factor inhibits neuronal terminal differentiation through STAT3 activation. Proc Natl Acad Sci USA 99:9015-20.

Morgan H D, Sutherland H G, Martin D I, and Whitelaw E (1999) Epigenetic inheritance at the agouti locus in the mouse. Nature Genetics 23: 314-8.

Morin-Kensicki E M, Faust C, LaMantia C, Magnuson T (2001): Cell and tissue requirements for the gene eed during mouse gastrulation and organogenesis. Genesis 31:142-6.

Mors O, Mortensen P B, Ewald H (2001): No evidence of increased risk for schizophrenia or bipolar affective disorder in persons with aneuploidies of the sex chromosomes. Psychol Med 31:425-30.

Mowry B J, Nancarrow D J (2001): Molecular genetics of schizophrenia. Clin Exp Pharmacol Physiol 28:66-9.

Mujaheed M, Corbex M, Lichtenberg P, et al (2000): Evidence for linkage by transmission disequilibrium test analysis of a chromosome 22 microsatellite marker D22S278 and bipolar disorder in a Palestinian Arab population. Am J Med Genet 96:836-8.

Myles-Worsley M, Coon H, McDowell J, et al (1999): Linkage of a composite inhibitory phenotype to a chromosome 22q locus in eight Utah families. Am J Med Genet 88:544-50.

Nan X, Campoy F J, Bird A. MeCP2 is a transcriptional repressor with abundant binding sites in genomic chromatin. Cell, 88(4):471-81, 1997.

Nan X, Ng H H, Johnson C A, Laherty C D, Turner B M, Eisenman R N, and Bird A (1998). Transcriptional repression by the methyl-CpG-binding protein MeCP2 involves a histone deacetylase complex. Nature 393: 386-9.

Nesic D, Kramer A (2001): Domains in human splicing factors SF3a60 and SF3a66 required for binding to SF3a120, assembly of the 17S U2 snRNP, and prespliceosome formation. Mol Cell Biol 21:6406-17.

Nurnberger J I Jr, Foroud T. Chromosome 6 workshop report. Am J Med Genet. Jun. 18, 1999;88(3):233-8.

Nurnberger J I Jr, Foroud T. Genetics of bipolar affective disorder. Curr Psychiatry Rep April 2000;2(2):147-57.

Petit J, Boisseau P, Taine L, Gauthier B, Arveiler B (1999): A YAC contig encompassing the 11q14.3 breakpoint of a translocation associated with schizophrenia, and including the tyrosinase gene. Mamm Genome 10:649-52.

Petronis A. Human morbid genetics revisited: relevance of epigenetics. Trends Genet. March 2001;17(3):142-6.

Petronis A, Paterson A D, Kennedy J L. Schizophrenia: an epigenetic puzzle? Schizophrenia Bulletin 25:4: 639-655, 1999

Petronis A. The genes for major psychosis: aberrant sequence or regulation? Neuropsychopharmacology, 23(1):1-12; 2000.

Petronis A, Gottesman, I I, Crow T J, et al (2000): Psychiatric epigenetics: a new focus for the new century. Mol Psychiatry 5:342-6.

Popendikyte V, Laurinavicius A, Paterson A D, Macciardi F, Kennedy J L, Petronis A. DNA methylation at the putative promoter region-of the human dopamine D2 receptor gene. Neuroreport 1999;10:1249-55.

Pulver A E, Karayiorgou M, Wolyniec P S, et al (1994): Sequential strategy to identify a susceptibility gene for schizophrenia: report of potential linkage on chromosome 22q12-q13.1: Part 1. Am J Med Genet 54:36-43.

Rakyan V K, Blewitt M E, Druker R, Preis J I, Whitelaw E (2002): Metastable epialleles in mammals. Trends Genet 18:348-51.

Razin, A. and Shemer, R. (1999) Epigenetic control of gene expression. Results Probl. Cell. Differ. 25, 189-204

Rice J P, Goate A, Williams J T, et al (1997): Initial genome scan of the NIMH genetics initiative bipolar pedigrees: chromosomes 1, 6, 8, 10, and 12. Am J Med Genet 74:247-53.

Riggs A, Xiong Z, Wang L, and LeBon J M (1998) Methylation dynamics, epigenetic fidelity and X chromosome structure. In: Wolffe A P (ed) Epigenetics, pp. 214-227. John Wiley & Sons, Chistester.

Robertson K D and Wolffe A P (2000) DNA methylation in health and disease. Nature Review Genet 1: 11-9

Rubin C M, VandeVoort C A, Teplitz R L, Schmid C W. Alu repeated DNAs are differentially methylated in primate germ cells. Nucleic Acids Research, 22(23):5121-7, 1994.

Ruprecht K, Kuhlmann T, Seif F, et al (2001): Effects of oncostatin M on human cerebral endothelial cells and expression in inflammatory brain lesions. J Neuropathol Exp Neurol 60:1087-98.

Schizophrenia Collaborative Linkage Group (1998): A transmission disequilibrium and linkage analysis of D22S278 marker alleles in 574 families: further support for a susceptibility locus for schizophrenia at 22q12. Schizophr Res 32:115-21.

Schmitt C, Tonnelle C, Dalloul A, Chabannon C, Debre P, Rebollo A (2002): Aiolos and Ikaros: Regulators of lymphocyte development, homeostasis and lymphoproliferation. Apoptosis 7:277-84.

Schramm M, Falkai P, Tepest R, Schneider-Axmann T, Przkora R, Waha A, Pietsch T, Bonte W, Bayer T A. Stability of RNA transcripts in post-mortem psychiatric brains. J Neural Transm. 1999;106(3-4):329:-35.

Schwab S G, Albus M, Hallmayer J, et al (1995a): Evaluation of a susceptibility gene for schizophrenia on chromosome 6p by multipoint affected sib-pair linkage analysis. Nat Genet 11:325-7.

Schwab S G, Hallmayer J, Albus M, et al (1998): Further evidence for a susceptibility locus on chromosome 10p14-p11 in 72 families with schizophrenia by nonparametric linkage analysis. Am J Med Genet 81:302-7.

Schwab S G, Lerer B, Albus M, et al (1995b): Potential linkage for schizophrenia on chromosome 22q12-q13: a replication study. Am J Med Genet 60:436-43.

Sherrington R, Brynjolfsson J, Petursson H, Potter M, Dudleston K, Barraclough B, Wasmuth J, Dobbs M, Gurling H. Localization of a susceptibility locus for schizophrenia on chromosome 5. Nature. Nov. 10, 1988;336(6195):164-7.

Shinozald A, Arahata K, Tsukahara T (1999): Changes in pre-mRNA splicing factors during neural differentiation in P19 embryonal carcinoma cells. Int J Biochem Cell Biol 31:1279-87.

Shinozaki A, Arahata K, Tsukahara T (1999): Changes in pre-mRNA splicing factors during neural differentiation in P19 embryonal carcinoma cells. Int J Biochem Cell Biol 31:1279-87.

Siegfried Z, Eden S, Mendelsohn M, Feng X, Tsuberi B Z, Cedar H. DNA methylation represses transcription in vivo. Nat Genet June 1999;22(2):203-206

Silva A J, White R. Inheritance of allelic blueprints for methylation patterns. Cell Jul. 15, 1988;54(2):145-52

Sinnett D, Richer C, Deragon J M, Labuda D. Alu RNA transcripts in human embryonal carcinoma cells. Model of post-transcriptional selection of master sequences. Journal of Molecular Biology, 226(3):689-706, 1992.

St Clair D, Blackwood D, Muir W, Carothers A, Walker M, Spowart G, Gosden C, Evans H J. Association within a family of a balanced autosomal translocation with major mental illness. Lancet. Jul. 7, 1990;336(8706):13-6.

Strack S, Robison A J, Bass M A, Colbran R J (2000): Association of calcium/calmodulin-dependent kinase II with developmentally regulated splice variants of the postsynaptic density protein densin-180. J Biol Chem 275:25061-4.

Straub R E, MacLean C J, Martin R B, et al (1998): A schizophrenia locus may be located in region 10p15-p11. Am J Med Genet 81:296-301.

Straub R E, MacLean C J, O'Neill F A, Walsh D, Kendler K S (1997): Support for a possible schizophrenia vulnerability locus in region 5q22-31 in Irish families. Mol Psychiatry 2:148-55.

Susser E, Neugebauer R, Hoek H W, et al (1996): Schizophrenia after prenatal famine. Further evidence. Arch Gen Psychiatry 53:25-31.

Thaker G K, Carpenter W T Jr. Advances in schizophrenia. Nat Med. June 2001; 7(6):667-71.

Tsuang M T, Stone W S, Faraone S V. Genes, environment and schizophrenia. Br J Psychiatry Supl. April 2001;40:s18-24.

Turnley A M, Stapleton D, Mann R J, Witters L A, Kemp B E, Bartlett P F (1999): Cellular distribution and developmental expression of AMP-activated protein kinase isoforms in mouse central nervous system. J Neurochem 72:1707-16.

Van Broeckhoven C, Verheyen G (1999): Report of the chromosome 18 workshop. Am J Med Genet 88:263-70.

van der Vlag J, Otte A P (1999): Transcriptional repression mediated by the human polycomb-group protein EED involves histone deacetylation. Nat Genet 23:474-8.

Verdoux H, Geddes J R, Takei N, et al (1997): Obstetric complications and age at onset in schizophrenia: an international collaborative meta-analysis of individual patient data. Am J Psychiatry 154:1220-7.

Verheyen G R, Villafuerte S M, Del-Favero J, et al (1999): Genetic refinement and physical mapping of a chromosome 18q candidate region for bipolar disorder. Eur J Hum Genet 7:427-34.

Viguera A C, Baldessarini R J, Hegarty J D, van Kammen D P, Tohen M. Clinical risk following abrupt and gradual withdrawal of maintenance neuroleptic treatment. Arch Gen Psychiatry 1997; 54(1):49-55

Wang S, Detera-Wadleigh S D, Coon H, et al (1996): Evidence of linkage disequilibrium between schizophrenia and the SCa1 CAG repeat on chromosome 6p23. Am J Hum Genet 59:731-6.

Wildenauer D B, Schwab S G. Chromosomes 8 and 10 workshop. Am J Med Genet. Jun. 18, 1999;88(3):239-43.

Xu G-L, Bestor T H. Nature Genetics 17: 376-379, 1997.

Yoder J A, Walsh C P, Bestor T H. Cytosine methylation and the ecology of intragenomic parasites. Trends Genetics, 13(8):335-40, 1997.

The present invention has been described with regard to preferred embodiments. However, it will be obvious to persons skilled in the art that a number of variations and modifications can be made without departing from the scope of the invention as described herein.

The embodiments of the invention in which an exclusive property of privilege is claimed are defined as follows:

1. A method of detecting an epigenetic abnormality associated with a disease comprising, identifying, within a eukaryotic genome, a locus having a hypomethylated sequence specific for said disease and an endogenous multi-copy DNA element.

2. The method of claim 1, wherein said step -of identifying comprises separate steps of identifying said disease-specific hypomethylated sequence and identifying said endogenous multi-copy DNA element.

3. The method of claim 2, wherein the steps may be performed in any order.

4. The method of claim 1, wherein said disease-specific hypomethylated sequence and said endogenous multi-copy DNA element are within 10 kilobases of separation.

5. The method of claim 1, wherein said endogenous multi-copy DNA element is a retroelement that is normally methylated.

6. The method of claim 5, wherein said retroelement is selected from the group consisting of endogenous retroviral sequences (ERV), SINE sequences, Alu sequences, LINE sequences, and L1 sequences.

7. A method of identifying a chromosomal region associated with a disease state comprising:
identifying a locus, within DNA obtained from said diseased sample, that has a DNA sequence that is hypomethylated and an endogenous multi-copy DNA element, wherein the DNA sequence is methylated in a non-disease sample and wherein the chromosomal region consists of from about 1 to about 10 DNA coding sequences that are proximal to the identified locus.

8. A method of identifying a DNA coding sequence having an epigenetically altered expression pattern that contributes to a disease in an organism comprising:
identifying a locus, within DNA obtained from said diseased sample, that has a DNA sequence that is hypomethylated and an endogenous multi-copy DNA element, said DNA sequence being methylated in a non-disease sample; and
comparing expression patterns of the DNA coding sequence that comprises, or that is located proximal to, said identified locus within said diseased sample and said non-diseased sample, to identify said DNA coding sequence having an epigenetically altered expression pattern.

9. The method of claim 8, wherein said disease is selected from the group consisting of Huntingdon's disease, schizophrenia, and bipolar disorder.

10. A method of diagnosing an epigenetic abnormality correlated with a disease comprising:
identifying a DNA sequence that is hypomethylated within a locus that has an endogenous multi-copy DNA element and is obtained from a diseased sample, said DNA sequence being methylated in a non-disease sample.

11. Method of detecting an epigenetic abnormality associated with a non-Mendelian disease, said method comprising:
a) extraction of genomic DNA from a sample that exhibits characteristics of a non-Mendelian disease;
b) digestion of said genomic DNA with a methylation-sensitive restriction enzyme to produce a pool of restricted DNA fragments;
c) fractionation of said pool of restricted DNA fragments to obtain DNA fragments of a desired size;
d) amplification of at least a segment of said DNA fragments of a desired size with primers that anneal to an endogenous DNA element to produce a PCR product;
e) cloning of said PCR product into a sequencing vector;
f) sequence determination of said PCR product to obtain a sequence of said PCR product;
g) comparing said sequence against a genomic database to assign a locus for said epigenetic abnormality associated with a non-Mendelian disease.

12. The method of claim 11, wherein said non-Mendelian disease is selected from the group consisting of schizophrenia, bipolar disorder, cancer, and diabetes.

13. The method of claim 11, wherein said sample that exhibits characteristics of a non-Mendelian disease is brain tissue.

14. The method of claim 13, wherein said sample that exhibits characteristics of a non-Mendelian disease is selected from the group consisting of frontal cortex and prefrontal cortex.

15. The method of claim 11, wherein said desired size is less than 10 kb.

16. The method of claim 11, wherein said endogenous DNA element is a multi-copy DNA element.

17. The method of claim 16, wherein said multi-copy DNA element is selected from the group consisting of endogenous retroviral sequence, LINE, SINE, L1, and Alu.

18. The method of claim 11, wherein said methylation-sensitive restriction enzyme is selected from the group consisting of AatII (GACGTC); Bsh1236I (CGCG); Bsh1285I (CGRYCG); BshTI (ACCGGT); Bsp68I (TCGCGA); Bsp119I (TTCGAA); Bsp143II (RGCGCY); Bsu15I (ATCGAT); Cfr10I (RCCGGY); Cfr42I (CCGCGG); CpoI (CGGWCCG); Eco47III (AGCGCT); Eco52I (CGGCCG); Eco72I (CACGTG); Eco105I (TACGTA); EheI (GGCGCC); Esp3I (CGTCTC); FspAI (RTGCGCAY); Hin1I (GRCGYC); Hin6I (GCGC); HpaII (CCGG); Kpn2I (TCCGGA); MluI (ACGCGT); NotI (GCGGCCGC); NsbI (TGCGCA); PauI (GCGCGC); PdiI (GCCGGC); Pfl23II (CGTACG); Psp1406I (AACGTT); PvuI (CGATCG); SalI (GTCGAC); SmaI (CCCGGG); SmuI (CCCGC); TaiI (ACGT); and TauI (GCSGC).

19. Method of identifying a gene having an epigenetically altered expression pattern that contributes to a non-Mendelian disease in an organism, said method comprising:
a) extraction of genomic DNA from a sample that exhibits characteristics of a non-Mendelian disease;
b) digestion of said genomic DNA with a methylation-sensitive restriction enzyme to produce a pool of restricted DNA fragments;
c) fractionation of said pool of restricted DNA fragments to obtain DNA fragments of a desired size;
d) amplification of at least a segment of said DNA fragments of a desired size with primers that anneal to an endogenous DNA element to produce a PCR product;
e) cloning of said PCR product into a sequencing vector;
f) sequence determination of said PCR product to obtain a sequence of said PCR product;
g) comparing said sequence against a genomic database to assign a locus for said epigenetic abnormality associated with a non-Mendelian disease;
h) searching said database to identify a gene located proximal to said locus;
i) comparing expression patterns of said gene located proximal to said locus within a test sample that exhibits characteristics of said non-Mendelian disease with expression patterns of a corresponding gene within a control sample to identify said gene having an epigenetically altered expression pattern.

20. A gene isolated by the method of claim 19.

21. Method of isolating a probe for detecting an epigenetic abnormality associated with a non-Mendelian disease, said method comprising:
a) extraction of genomic DNA from a sample that exhibits characteristics of a non-Mendelian disease;
b) digestion of said genomic DNA with a methylation-sensitive restriction enzyme to produce a pool of restricted DNA fragments;
c) fractionation of said pool of restricted DNA fragments to obtain DNA fragments of a desired size;
d) amplification of at least a segment of said DNA fragments of a desired size with primers that anneal to an endogenous DNA element to produce a PCR product;
e) using said PCR product as said probe to detect said epigenetic abnormality associated with a non-Mendelian disease in another sample.

22. A probe isolated by the method of claim 21.

23. A method of detecting a disease associated with an epigenetic abnormality comprising, identifying, within a eukaryotic genome, a locus having a hypomethylated sequence specific for said disease and an endogenous multi-copy DNA element.

24. A method of diagnosing a disease correlated with an epigenetic abnormality comprising:
identifying a DNA sequence that is hypomethylated within a locus that has an endogenous multi-copy DNA element and is obtained from a diseased sample, said DNA sequence being methylated in a non-disease sample.


SEQUENCE LISTING (263 sequences)

SEQ ID NO: 1 - DNA, 148 bp, Homo sapiens
  misc_feature: consensus sequence of a subfamily of Alu repeats having accession number U14570
ggccgggcgcggtggctcacgcctgtaatcccagcactttgggaggccgaggcgggtggatcatgaggtcaggagatcgagaccatcctggctaacaaggtgaaaccccgtctctactaaaaatacaaaaaattagccgggcgcggtg

SEQ ID NO: 2 - DNA, 20 bp, Artificial
  primer ′Alu For′ (see Example 1)
gcctgtactcccagcagttt

SEQ ID NO: 3 - DNA, 20 bp, Artificial
  primer ′Alu Rev′ (see Example 1)
ggagggtgtttgcacaatct

SEQ ID NO: 4 - DNA, 24 bp, Artificial
  primer ′1MF′ (see Example 2)
cagcgtacacatacacagaagaga

SEQ ID NO: 5 - DNA, 23 bp, Artificial
  primer ′1MR′ (see Example 2)
ttcctagtcaccaagtcatagca

SEQ ID NO: 6 - DNA, 109 bp, Homo sapiens
  misc_feature: Alu sequence cloned from Sch74-E52m/Sch74-E51m (see Figure 4)
cagctcactgcaacctccgcctcctggattcaagcgattttcccgccttagcctcctgagtaactgggactagaggcaggtaccaccacgcccagctaatttttgtatt

SEQ ID NO: 7 - DNA, 109 bp, Homo sapiens
  misc_feature: Alu sequence cloned from BD43-E78m/BD43-E83m (see Figure 4)
cggctcaatgcaacctcagcctcctgggttcaagcaattctcctgtctcagcctcccgagtagctgggattacaggcacatgccaccatgcccaactaatttttgtatt

SEQ ID NO: 8 - DNA, 109 bp, Homo sapiens; identical to SEQ ID NO: 7
  misc_feature: Alu sequence cloned from BD43-E78m (see Figure 4)

SEQ ID NO: 9 - DNA, 109 bp, Homo sapiens
  misc_feature: Alu sequence cloned from BD34-D19M (see Figure 4)
tggctcactgtaacctctgcctcctgggttcaagtaattctcctgtctcagcctcctgagtagctaggattactggtgcccgccaccatgcccggcaaatttttgtatt

SEQ ID NO: 10 - DNA, 109 bp, Homo sapiens; identical to SEQ ID NO: 9
  misc_feature: Alu sequence cloned from BD34-E62m_ (see Figure 4)

SEQ ID NO: 11 - DNA, 109 bp, Homo sapiens
  misc_feature: Alu sequence cloned from BD43-15m (see Figure 4)
tggctcactgcaacctctacctcctgagttcaagctcttctcctgcctcaacctccagagtaattgtgattacaggtgcctcccaccacaccaggctaatttttgtatt

SEQ ID NO: 12 - DNA, 109 bp, Homo sapiens
  misc_feature: Alu sequence cloned from Ctrl57-E5m (see Figure 4)
cagctcactgcaacctccatttcctgggttcaagcgattctcctgcctcagcctccggagtagctgggaccacagacgtgtgccaccatgcctgggtaattttcatatt

SEQ ID NO: 13 - DNA, 110 bp, Homo sapiens
  misc_feature: Alu sequence cloned from BD43-RevE7m (see Figure 4)
cagctcactgcaaccaccacctcccaggttcaagtgattatcctgcctcagcctcccgagtagctgggattacagatgcccaccaacacaccaggctaattttttgtatt

SEQ ID NO: 14 - DNA, 110 bp, Homo sapiens; identical to SEQ ID NO: 13
  misc_feature: Alu sequence cloned from BD43-RevE77m (see Figure 4)

SEQ ID NO: 15 - DNA, 110 bp, Homo sapiens
  misc_feature: Alu sequence cloned from BD34-A14M (see Figure 4)
cagcccagtgcaagctccgcctcccaggttcacgtcattctcctgcctcagcctcccgagtagctgggactacaggcgcccgccaccacgcccagctaattttttgtatt

SEQ ID NO: 16 - DNA, 110 bp, Homo sapiens
  misc_feature: Alu sequence cloned from Ctrl57-E3m (see Figure 4)
cggctcactgcaagctccgcctcccgggtgcacgccattctcctgcctcagcctcccgagtagctgggactacaggcgcccgccaccacgcccggctaattttttgtatt

SEQ ID NO: 17 - DNA, 110 bp, Homo sapiens
  misc_feature: Alu sequence cloned from Sch74-E318m (see Figure 4)
tggctcactgcaacctccgcctcccaggttcaagcaattctcctgcctcagtctcccgagtagctgggactaccggcgagtgctaccatgcctgcgtaattttttgtact

SEQ ID NO: 18 - DNA, 110 bp, Homo sapiens; identical to SEQ ID NO: 17
  misc_feature: Alu sequence cloned from Sch74-E318_m (see Figure 4)

SEQ ID NO: 19 - DNA, 110 bp, Homo sapiens; identical to SEQ ID NO: 17
  misc_feature: Alu sequence cloned from Ctrl57-E4m (see Figure 4)

SEQ ID NO: 20 - DNA, 110 bp, Homo sapiens
  misc_feature: Alu sequence cloned from BD43-E79m (see Figure 4)
cagctcactgcaacctccgtttcccaggtgcaaccgattctcctgcctcagacctctgaagcggctgggactacaggtgcctgccacctcacccggctaatttttgtatt

SEQ ID NO: 21 - DNA, 108 bp, Homo sapiens
  misc_feature: Alu sequence cloned from Ctrl57-E6m (see Figure 4)
cagctcaccacaacctccgcctcctgggttccagcgattctcctgcctcggcctcccaagtagctgggattacaggcacgcaccaatacacctggctaattttgtatt

SEQ ID NO: 22 - DNA, 108 bp, Homo sapiens; identical to SEQ ID NO: 21
  misc_feature: Alu sequence cloned from Ctrl57-E6m (see Figure 4)

SEQ ID NO: 23 - DNA, 110 bp, Homo sapiens
  misc_feature: Alu sequence cloned from Ctrl50-RevE169m (see Figure 4)
cagctctccacaacctccgccatcgtgggttccagcagattctcctgcctcggcctcccaagtagctgggaatacaggcacgctccaatacacctggctaattatgtatt

SEQ ID NO: 24 - DNA, 109 bp, Homo sapiens
  misc_feature: Alu sequence cloned from Sch56-E283m (see Figure 4)
cagctcaccgcaacctttgcctcacgggctcaagtgattctcatgcttgatcctaccaagtagctgggattacaggcacatgccatcatgctgagctaactttggtatt

SEQ ID NO: 25 - DNA, 109 bp, Homo sapiens; identical to SEQ ID NO: 24
  misc_feature: Alu sequence cloned from Sch56-r-37m (see Figure 4)

SEQ ID NO: 26 - DNA, 105 bp, Homo sapiens
  misc_feature: Alu sequence cloned from Sch56-E32m (see Figure 4)
cacgtcactgtaatgtccatctcccgggttcaggtgattctcctgccccagcctcctgagtagctgtacaggcgtgcaccaccatgcccgactaatttttgtact

SEQ ID NO: 27 - DNA, 98 bp, Homo sapiens
  misc_feature: Alu sequence cloned from Ctrl50-E166m (see Figure 4)
cggcccactgcaacctccgcctcccgggtgcaagcagttctcctacctcagcctcctgagtagctaggattacaggcacacctggctaattttgtggt

SEQ ID NO: 28 - DNA, 110 bp, Homo sapiens
  misc_feature: Alu sequence cloned from Ctrl50-E49m (see Figure 4)
cgactcattgcaacctctgcctcctgggtttaagccgttctcatgcctcagcctcccgacgtagctgggattataggcatgcgccaccacccccagctaatttttgtatt

SEQ ID NO: 29 - DNA, 589 bp, Homo sapiens
  misc_feature: Alu sequence cloned from E-130_m37_SZ (see Figure 3)
ctgattacgccaagctctaatacgactcactatagggaaagctcggtaccacgcatgcttgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcccagctggcgtaatagacgaagaggcccgcaccgatcgcccttcccaacagttgcgcaagcctg

SEQ ID NO: 30 - DNA, 612 bp, Homo sapiens
  misc_feature: Alu sequence cloned from E-140_m48_SZ (see Figure 3)
ctatcccatgattacgccaagctctaatacgactcactatagggaaagctcggtaccacgcatgctgcagacgcgttacgtatcggatccagaattcgtgattgcctgtactcccagcagtttgggaggctgaggtaggtggatcacgaggtcaggagttctagatcagcctggccaacagggtgaaaccatgtctctactaaaaatacaaaaattagtcaggcgtggtggtgggcacctgtaatcccagttacttgggaggctgaggcaggagaatttcttgaacctggaaggcagaggttgcagtcagccgagattgtgcaaacaccctccaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaacctggcgttacccaacttaatcgccttgcagcacatccccctttcgccagctggcgtaatagcgaagaggcccgcaccgatcgcccttcccacagttgcgcagcctgaatggcgaatggaaattgtaa

SEQ ID NO: 31 - DNA, 602 bp, Homo sapiens
  misc_feature: Alu sequence cloned from E-150_m48_SZ (see Figure 3)
ctatgaccatgattacgccaagctctaatacgactcactatagggaaagctcggtaccacgcatgctgcagacgcgttacgtatcggatccagaattcgtgattgcctgtactcccagcagtttgggaggccaaatcagatggatcatctgaggtcaggagttcaagaaccaccttatcaacatgaagaatcctggtctctactaaaagtacaaaattagccaggtatcatggcaaatgcttgtcatcctagctactcagaaggctgaggcagaggaatcacttgaacctgtgaggcggaggtttcggtgagctgagattgtgcaaacaccctccaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagagggccgcaccgatcgcccttccaacagttgcgcagcctgaatggcga

SEQ ID NO: 32 - DNA, 620 bp, Homo sapiens
  misc_feature: Alu sequence cloned from E-154_m56_SZ (see Figure 3)
atgattacgccaagctctaatacaactcactatgggcaaatggtcgcaacctcgcatgctgcatacgcgttacgtatcggatccagaattcgtgattggagggtgtttgcacaatctcagctcactgcaacctccacctcccaggctcaatgatcctcccacctcaactcccccgagtaactgggaccacaggtgcatgccagcatgcccagctaatttttgtattttctgttgagatggggttttgccatgttgcccaggcaggtctcgaactgctgggctcaagtgatcctcctgcctccacctcacaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatat

SEQ ID NO: 33 - DNA, 598 bp, Homo sapiens
  misc_feature: Alu sequence cloned from E-178_m74_SZ (see Figure 3)
aagatccatatgaccatgattacgccaagctctaatacgactcactatagggaaagctcggtaccacgcatgctgcagacgcgttacgtatcggatccagaattcgtgattggagggtgtttgcacaatcttggctcactgcaacctccgcctcccgggttcaagagattctcctgcctcagcctcccgagaggctgggactacaggcatgcgccaccatgcccagctagtttttgtatttttagtagagatggggtttccccatgttggccaggatgatctcgatctcttgacctcgtgatctgcccgcctcagcctcccaaacttgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcag

SEQ ID NO: 34 - DNA, 692 bp, Homo sapiens
  misc_feature: Alu sequence cloned from E-191_m34-4_BD (see Figure 3)
atgattacgccaagctctaatacgactcactatagggaaagctcggtaccacgcatgctgcagacgcgttacgtatcggatccagaattcgtcgatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgtttgttccagtttggaacaagagtccactattaaagaacgtggactccaacgtcaaagggcgaaaaaccgtctatcagggcgatggcccactacgtgaaccatcaccctaatcaagtttttggggtcgaggtgccgtaaagcactaaatcggaaccctaaagggagcccccgatttagagcttgacggggaaagc

SEQ ID NO: 35 - DNA, 530 bp, Homo sapiens
  misc_feature: Alu sequence cloned from E-221_m37_SZ (see Figure 3)
ccatatgaccatgattacgccaagctctaatacgactcactatagggaaagctcggtaccacgcatgctgcagacgcgttacgtatcggatccagaattcgtgattgcctgtactcccagcagtttgggaggccaaatcagatggatcatctgaggtcaggagttcaagaaccaccttatcaacatgaagaatcctggtctctactaaaaatacaaaattagccaggtatcatggcaaatgcttgtcatcctagctactcagaaggctgaggcagaggaatcacttgaacctgtgaggcggaggtttcggtgagctgagattgtgcaaacaccctccaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccc

SEQ ID NO: 36 - DNA, 600 bp, Homo sapiens
  misc_feature: Alu sequence cloned from E-244_m48_SZ (see Figure 3)
ccgtatgaccatgattacgccaagctctaatacgactcactatagggaaagctcggtaccacgcatgctgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggccgcaccgatcgcccttcccaacagttgcgcagcctgaatg

SEQ ID NO: 37 - DNA, 586 bp, Homo sapiens
  misc_feature: Alu sequence cloned from E-246_m48_SZ (see Figure 3)
ctatgaccatgattacgccaagctctaataccgactcactatagggaaagctcggtaccacgcatgctgcagacgcgttacgtatcggatccagaattcgtgattggagggtgtttgcacaatctcggctcactgcaacctccacctcccaggttcaagcaattctcctgcctcagcctcccaagtagctgagattacaggcggctgccatcatgcctggctaatttttgtatttttactaaagacggggttttgccatgttggccaggctgttctcaaactcctgacttcaggtgatccacctgcctcagcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagt

SEQ ID NO: 38 - DNA, 560 bp, Homo sapiens
  misc_feature: Alu sequence cloned from E-251_m48_SZ (see Figure 3)
catgattacgccaagctctaatacgactcactatagggaaagctcggtaccacgcatgctgcagacgcgttacgtatcggatccagaattcgtgattcggagggtgtttgcacaatcttgactaactgcaacatctgcctcccaggttcaagcaattctgcctcagcttcctgagcagctgggattacagatgagcactaccatgacaggctaatttttatatttttagtagaggcggggtttcaccatgttggccaggctggtcatgaactcctgacctcaggtgattcacctgcctcagcctcccaaactgctgggaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcc

SEQ ID NO: 39 - DNA, 581 bp, Homo sapiens
  misc_feature: Alu sequence cloned from E-252_m48_SZ (see Figure 3)
cgatatgaccatgattacgccaagctctaatacgactcactatagggaaagctcggtaccacgcatgctgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactggggaaaaccctggcgttacccaacttaatcgccttgcagcacatccccctttcgccagctggcgtaatagcgaagaggcccgcaccgatcgcccttccc

SEQ ID NO: 40 - DNA, 571 bp, Homo sapiens
  misc_feature: Alu sequence cloned from E-2531_m48_SZ (see Figure 3)
cagctatgaccatgattacgccaagctctaatacgactcactatagggaaagctcggtaccacgcatgctgcagacgcgttacgtatcggatccagaattcgtgattgcctgtactcccagcagtttgggaggctgaggcaggtgaatcacctgaggtcaggagttcatgaccagcctggccaacatggtgaaaccccgcctctactaaaaatataaaaattagcctgtcatggtagtgctcatctgtaatcccagctgctcaggaagctgaggcagaattgcttgaacctgggaggcagatgttgcagttagtcaagattgtgcaaacaccctccaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagagggccgcaccgatcg

SEQ ID NO: 41 - DNA, 599 bp, Homo sapiens
  misc_feature (575)..(575): n is a, g, c, or t
ctatgaccatgattacgccaagctctaatacgactcactatagggaaagctcggtaccacgcatgctgcagacgcgttacgtatcggatccagaattcgtgattgcctgtactcccagcagtttgggaggctgaggcaggtgaatcacctgaggtcaggagttcatgaccagcctggccaacatggtgaaaccccgcctctactaaaaatataaaaattagcctgtcatggtagtgctcatctgtaatcccagctgctcaggaagctgaggcagaattgcttgaaccttgggaggcagatgttgcagttagtcaagattgtgcaaacaccctccaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttnccaacagttgcgcagcctgaatgg

SEQ ID NO: 42 - DNA, 500 bp, Homo sapiens
  misc_feature: Alu sequence cloned from E-258_m48_SZ (see Figure 3)
ccatatgatcatgattacgccaagctctaatacgactcactatagggaaagctcggtaccaccgcatgctgcagacgcgttacgtatcggatccagaattcgtgattggagggtgtttgcacaatcttggctcactgcaacctctgccccccaggttcaaacgattctcctgcctcagcctcccgagtagctgggattataggcacctgccaccacgcccagctaattttttgcatttttagtagagacggggtttcactatgttggccaggctggtctagaactcctgaccttgtgatccgcccgccttggcctcccaaactgctgggagtaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaac

SEQ ID NO: 43 - DNA, 510 bp, Homo sapiens
  misc_feature: Alu sequence cloned from E-261_m50_Ctrl (see Figure 3)
tgaccttgattacgccaagctctaatacgactcactatagggaaagctcggtaccacgcatgctgcagacgcgttacgtatcggatccagaattcgtgattggagggtgtttcgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgcc

SEQ ID NO: 44 - DNA, 520 bp, Homo sapiens
  misc_feature: Alu sequence cloned from E-267_m50_Ctrl (see Figure 3)
ttacgccaagctctaatacgactcactatagggaaagctcggtaccacgcatgctgcagacgcgttacgtatcggatccagaattcgtgattgcctgtactcccagcagtttgggaggccaaatcagatggatcatctgaggtcaggagttcaagaaccaccttatcaacatgaagaatcctggtctctactaaaaatacaaaattagccaggtatcatggcaaatgcttgtcatcctagctactcagaaggctgaggcagaggaatcacttgaacctgtgaggcggaggtttcggtgagctgagattgtgcaaacaccctccaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctgggcgttacccaacttaatcgccttgcagcacatcccccttt

SEQ ID NO: 45 - DNA, 355 bp, Homo sapiens
  misc_feature (11)..(11): n is a, g, c, or t
cttccaaaggntaagntctaatattactcactatagggaaagctcggccccactcatgctgcagacgcgttacgtattggatccagaattcgcgattggagggtgtttgtacaatctctgctcaccgaaacctccgcctcacaggttcaagtgatccctctgcctcagccttctgagtagctaggatgacaagcatttgccatgatacctggctaattttgtatttttagtagagaccaggattcttttatgttgataaggcggttcttgaactcctgacctcagattgattcatctgatttggcctcccaaactgctgggagtacaggcaatctgaattcgtcaacaagcttct

SEQ ID NO: 46 - DNA, 601 bp, Homo sapiens
  misc_feature: Alu sequence cloned from E-285_m56_SZ (see Figure 3)
ggtgagagattacgccaagctctaatacgactcactatagggaaagctcggtaccacgcatgctgcagacgcgttacgtatcggatccagaattcgtgattgcctgtactcccagcagtttgggaggctgaagtgggttgattacccgaggtcaggagttccagaccaggttgaccaacatggagaaaccctgtctctactaaaaatacaaaattagccaggtgtattggtgcgtgcctgtattcccagctacttgggaggccgaggcaggagaatcgctggaacccaggaggcggaggttgtggtgagctgagattgtgcaaacaccccccaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaataagcgaagaggcccgcaccgatcgccctttccaacagttgcgcaagcctgaatggcg

SEQ ID NO: 47 - DNA, 600 bp, Homo sapiens
  misc_feature: Alu sequence cloned from E-286_m56_SZ (see Figure 3)
gttctaatacgactcactatagggaaagctcggtaccacgcatgctgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggcccgcacaattcactggccgtcgttttacaacgtcgtgactgggaaaaccctggcgttacccaacttaatcgccttgcagcacatccccctttcgccagctggcgtaatagcgaagaagcccgcaccgatcgcccttcccaacagttgcgcagcctgaatggcgaatggaaattgtaagcgt

SEQ ID NO: 48 - DNA, 400 bp, Homo sapiens
  misc_feature: Alu sequence cloned from E-287_m56_SZ (see Figure 3)
taattaactcactatagggaaagctcgggagcacgcatgctgcatacgcgtttcgtatctggatccagaattcgcgattgcctgtactcccagcagtttgggaggccaaatcagatggatcatctgaggccaggagttcaagaaccaccttatcaacatgaataatcctggtctctactaaaaatacgaaattagccaggtatcatggaaaatgcttgtcatcctagctactcagaaggctgaggcagaggaatcacttgaacctgtgaggcggaggtttcggtgagctgagattgggcaaacaccctccaatctgaattcgtccgacaagcttctcgagcctaggctagctctagaccacacgcgtgggggcccgagctcgcggccgctgtattctatt

SEQ ID NO: 49 - DNA, 453 bp, Homo sapiens
  misc_feature (15)..(15): n is a, g, c, or t
gttcagatctaatangactcactatcgggaaagctcggcaccacgcatgctgcagacgcgttacgtatccggatccatgaattcgtgattgcctgtactcccagcagtttgggaggccaaatcagatggatcatctgaggtcaggagttcaagaaccaccttatcaacatgaagaatcctggtctctactaaaaatacaaaattagccaggtatcatggcaaatgcttgtcatcctagctactcagaaggctgaggcagaggaatcacttgaacctgtgaggcggaggtttcggtgagctgagattgtgcaaacaccctccaatctgaattcgtcgacaagcttctcgagcctaggctagctctagaccacacgtgtgggggcccgagctcgcggccgctgcattctatagtgtcacctaaatggccgcacaattcactggccgtcgttttta

SEQ ID NO: 50 - DNA, 601 bp, Homo sapiens
  misc_feature: Alu sequence cloned from E-289_m56_SZ (see Figure 3)
ttacgccaagctctaatacgactcactatagggaaagctcggtaccacgcatgctgcagacgcgttacgtatcggatccagaattcgtgattgcctgtactcccagcagtttgggaggccaaatcagatggatcatctgaggtcaggagttcaagaaccaccttatcaacatgaagaatcctggtctctactaaaaatacaaaattagccaggtatcatggcaaatgcttgtcatcctagctactcagaaggctgaggcagaggaatcacttgaacctgtgaggcggaggtttcggtgagctgagattgtgcaaacaccctccaatctgaattcgtcgacaagcttctcgagcctaggctagctctagaccacacgtgtgggggcccgagctcgcggccgctgtattctatagtgtcacctaaatggccgcacaattcactgggccgtcgttttacaacgtcgtgactgggaaaaccctggcgttacccaacttaatcgccttgcagcacatccccctttcgccagctggcgtaatagcgaagaggccgcaccgatcgcccttcccaacagttgcgcagcctgaatggcgaatggaaattg

SEQ ID NO: 51 - DNA, 580 bp, Homo sapiens
  misc_feature: Alu sequence cloned from E-290_m56_SZ (see Figure 3)
atattgatcatgattacgccaacgctctaatacgactcactatagggaaagctcggtaccacgcatgctgcagacgcgttacgtatcggatccagaattcgtgattgcctgtactcccagcagtttgggaggctgaagtgggttgattacccgaggtcaggagttacagaccaggttgaccaacatggagaaaccctgtctctactaaaaatacaaaattagccaggtgtattggtgcgtgcctgtaatcccagctacttgggaggccgaggcaggagaatcgctggaacccaggaggcggaggttgtggtgagctgagattgtgcaaacaccctccaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttccc

SEQ ID NO: 52 - DNA, 579 bp, Homo sapiens
  misc_feature (469)..(469): n is a, g, c, or t
tgaccatgattacgccaagctctaatacgactcactatagggaaagctcggtaccacgcatgctgcagacgcgttacgtatcggatccagaattcgtgattggagggtgtttgcacaatctcagctcaccgaaacctccgcctcacaggttcaagtgattcctctgcctcagccttcagagtagctaggatgacaagcatttgccatgatacctggctaattttgtatttttagtagagaccaggattcttcatgttgataaggtggtccttgaactcctgacctcagatgatccatctgatttggcctcccaaactgctgggagtacaggcaatctgaattcctcgacaagcttctcgagcctaggctagctctagaccacaccgtgtgggggcccgagctcgcggccgctgtattctatagtgtcacctaaatggccgcacaattcactggccgtcgttttacaacntcgtgactgggaaaaccctgncgttaccccacttaatcncccttgcagcacatccccctttcgcccagnctgggcgtaatnancgaanaggcccgcacccgatcgcccct

SEQ ID NO: 53 - DNA, 530 bp, Homo sapiens
  misc_feature: Alu sequence cloned from E-292_m56_SZ (see Figure 3)
acgtcacgctctaatacgactcactatagggaaagctcggtaccacgcatgctgcagacgcgttacgtatcggatccagaattcgtgattgcctgtactcccagcagtttgggagggcaaatcagatggatcatctgaggtcaggagttcaagaaccaccttatcaacatgaagaatcctggtctctactaaaaatacaaaattagccaggtatcatggcaaatgcttgtcatcctagctactcagaaggctgaggcagaggaatcacttgaacctgtgaggcggaggtttcggtgagctgagattgtgcaaacaccctccaatctgaattcgtcgacaagcttctcgagcctaggctagctctagaccacacgttgtgggggcccgagctcgcggccgctgtattctatagtgtcacctaaatgggcgcacaattcactggccgtcgttttacaacgttcgtgactgggaaaaccctggcgttacccaacttaatcgcctttgcagcacatcccccctttcgcccagct

SEQ ID NO: 54 - DNA, 600 bp, Homo sapiens
  misc_feature: Alu sequence cloned from E-293_m56_SZ (see Figure 3)
tatgaccatgattacgccaagctctaatacgactcactatagggaaagctcggtaccacgcatgcttgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactgggccgtcgttttacaacgtcgtgactgggaaaaccctggcgttacccaacttaatcgccttgcagcacatccccctttcgccagctggcgtaatagcgaagaggccgcacccgatcgcccttcccaacagttgcgcagcctgaatg

SEQ ID NO: 55 - DNA, 580 bp, Homo sapiens
  misc_feature: Alu sequence cloned from E-294_m740_SZ (see Figure 3)
ttacgccacgctctaatacgactcactatagggaaagctcggtaccacgcatgctgcagacgcgttacgtatcggatccagaattcgtcgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcag

SEQ ID NO: 56 - DNA, 600 bp, Homo sapiens
  misc_feature: Alu sequence cloned from E-295_m740_SZ (see Figure 3)
tatgaccatgattacgccaagctctaatacgactcactatagggaaagctcggtaccacgcatgcttgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatgggccgcacaattcactgggccgtcgttttacaacgtcgtgactgggaaaaccctggcgttacccaacttaatcgccttgcagcacatccccctttcgccagctggcgtaatagcgaagaggcccgcaccgatcgcccttcccaacagtttgcgcagcctgaa

SEQ ID NO: 57 - DNA, 520 bp, Homo sapiens
  misc_feature: Alu sequence cloned from E-296_m57_Ctrl (see Figure 3)
caagctctaatacgactcactatagggaaagctcggtaccacgcatgctgcagacgcgttacgtatcggatccagaattcgtgattggagggtgtttgcacaatctcagctcactgcaacctctgcctcctgggttcaattcattctcctgcctcagccttccgagtagctgggattacaggcatgcccggctaatttttgtatttttagcagagatcggggttttgccatgttgcccaggctggtctcgaactcctaaccttgtgatctgcccacctcggcctcccaaactgctgggagtacaggcaatctgaattcgtcgacaagcttctcgagcctaggctagctctagaccacacgtgtgggggcccgagctcgcggccgctgtattctatagtgtcacctaaatgggccgcacaattcactgggcccgtcgttttacaacgtcgtgactgggaaaaccctgggcgttacccaacttaatcgcccttgcagcacatccccctttcgccagcttggc

SEQ ID NO: 58 - DNA, 610 bp, Homo sapiens
  misc_feature: Alu sequence cloned from E-297_m740_SZ (see Figure 3)
tatgaccatcattacgccaagctctaatacgactcactatagggaaagctcggtaccacgcatgctgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggccgcaccgatcgcccttcccaacagttgcgcagcctgaatggcgaatggaaatt

SEQ ID NO: 59 - DNA, 499 bp, Homo sapiens
  misc_feature: Alu sequence cloned from E-298_m57_Ctrl (see Figure 3)
gtcccgatctaatacgactcactatagggaaagctcggtaccacgcatgctgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatcgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcaccctaaatggccgcacaattcactgggccgtcgttttacaacgtcgtgactgggaaaaccctgggcgttaccccaacttaatcg

SEQ ID NO: 60 - DNA, 383 bp, Homo sapiens
  misc_feature (368)..(368): n is a, g, c, or t
gtcaagatcgaataggactcactatagggaaagctcggtaccacgcatgctgccgacgcgttacgtatcggatccagaattcgtgattgcctgtactcccagcactttgggagggcaaatcagatggatcatctgaggtcaggagttcaagaaccatccttatcaacatgaagaatcctggtctctactaaaaatacaacattagccaggtatcatggcaaatgcttgtcatcctagctactcacaaggctgaggcagaggaatcacttgaacctgtgaggcgcaggtttcggtgagctgagattgtgcaaacaccctccaatctgaattcgtcgacaagctctctcgagcctaggctagctttagancacacgtgtgggggc

SEQ ID NO: 61 - DNA, 360 bp, Homo sapiens
  misc_feature: Alu sequence cloned from E-300_m57_Ctrl (see Figure 3)
gttgaaacggcaagatctaatacgactcactatagggaaagctcggcactacgcatgctgcagacgtgttgacgtatcggatccagaattcgtgattggagggcgtttgcgcaatcttgactaactgcaacatctgcctcccaggctcaagcaattctgcctcagttttctgagcagctgggattacagatgagcactaccatgacaggctaatttttatatttttactagaggcggggattcaccatgtcggccaggttggtcatgaactcctgacctcaggcgattcacctgcctccgcctcccaaactgctgggagtacaggcaatctgaattcgtcgacaagcttctcgagcctag

SEQ ID NO: 62 - DNA, 526 bp, Homo sapiens
  misc_feature: Alu sequence cloned from E-304_m57_Ctrl (see Figure 3)
ctacgtacgctctaatacgactcactatagggaaagctcggtaccacgcatgctgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggcggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggcccgcacaattcactggccgtcgttttacaacgtcgtgactgggaaaaccctggcgttacccaacttaatcgccttgcagcacatccccctttcgccagc

SEQ ID NO: 63 - DNA, 460 bp, Homo sapiens
  misc_feature: Alu sequence cloned from E-305_m740_SZ (see Figure 3)
ttacgccaagctctaatacgactcactatagggaaagctcggtaccacgcatgctgcagacgcgttacgtatcggatccagaattcgcgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaatctgaattcgtcgacaagcttctccgagcctaggctagctctagaccacacgtgtgggggccgagctcgcggccgctgtattctatagtgtcacctaaatggccgcacaattcactggccgtcgtttttacaacg

SEQ ID NO: 64 - DNA, 452 bp, Homo sapiens
  misc_feature: Alu sequence cloned from E-308_m74_SZ (see Figure 3)
ttacgtcaagctctaatacgactcactatagggaaagctcggtaccacgcatgctgcagacgcgttacgtatcggatccagaattcgtgattggagggtgtttgcacaatctcagctcaccgaaatctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttt

SEQ ID NO: 65 - DNA, 419 bp, Homo sapiens
  misc_feature: Alu sequence cloned from E-309_m74_SZ (see Figure 3)
aggcaagatctaatacgactcactatagggaaacgctcggtaccacgcatgctgcagacgcgttacgtatcggatccagaattcgtgattgcctgtactcccacgcagtttgggaggccaaatcagatggatcatctgaggtcaggagttcaagaaccaccttatcaacatgaagaatcctggtctctactaaaaatacaacattagccaggtatcatggcaaatgcttgtcatcctagctactcagaaggctgaggcagaggaatcacttgaacctgtgaggcggaggtttcggtgagctgagattgcgcaaacaccctccaatctgaattcctctgacaagcttctcgagcctaggctagctctagaccccacgtgtgggggcccgagctcgccgtcgctgtatttctatagtcgtc

SEQ ID NO: 66 - DNA, 500 bp, Homo sapiens
  misc_feature: Alu sequence cloned from E-310_m74_SZ (see Figure 3)
ttacgtcaccgctctaatacgactcactatagggaaagctcggtaccacgcatgctgcagacgcgttacgtatcggatccagaattcgtgattggagggtgtttgcacaatctcagctcactgcaacctctgcctctcaggttcaagtgattctcctgcctcatcctccccagtagctgggtttacaggcatgcaccaccacagctggctaatttttgtatttttagtagagatggggtttcaccatgttggacaggctagtcttgaactcctgacctcaagtgatccacccgcctcagcctctcaaactgctgggagtacaggcaatctgaattcgtcgacaagcttctcgagcctaggctagctctagaccacacgtgtgggggcccgagctcgcggccgctgtattctatagtgtcacctaaatgggccgcacaattcactggccgtccgttttacaacgtccgtgactgggaaaaccctggcgttacccaacttaa

SEQ ID NO: 67 - DNA, 480 bp, Homo sapiens
  misc_feature: Alu sequence cloned from E-311_m74_SZ (see Figure 3)
aaacgccaagctctaatacgactcactatagggaaagctcggtaccacgcatgctgcagacgcgttacgtatcggatccagaattcgtgattgcctgtactcccagcagtttgggaggccgaggtgggtggatcacctgaggctgagagttcgagaccagcctagccaacatggtgaaaccctgtctctactaaaaatacaaaaattagccaggcaaggcagcacacgcctgtaattccacctactcgggatgctgaggcatgagaatcgcttgaacctgggaggtggagcttgcagtgaactgagattgtgcaaacaccctcaatctgaattcgtcgacaagcttctcgagcctaggctagctctagaccacacgtgtgggggcccgagctcgccggccgctgtattctattagtgtcacctaaatgggccgcacaattcactggccgtccgttttacaacgtcgtgactgggaaaacc

SEQ ID NO: 68 - DNA, 390 bp, Homo sapiens
  misc_feature: Alu sequence cloned from E-312_m74_SZ (see Figure 3)
cgaatacgactactatacggaaagctcggtaccacgcatgctgcacacgcgttacgcatcggatccagaattcgtgattgcctgtactcccagcagtttgggagggcaaatcagatggatcatctgaggtcaggagttcaagaaccaccttatcaacatgaagaatcctggtctctactaaaaatacaaaattagccaggtatcatcggcaaatgcttcgtcatcctagctactcagaaggctgaggcagaggagtcacttgaacctgtgaggcggaggaaacggcgagatgagattgtgcaaacaccctccaatttgaaattcgtcgacaagcttctccgagctctaggctagctctagacccacacgtgtgggggccccgagctcgcg

SEQ ID NO: 69 - DNA, 547 bp, Homo sapiens
  misc_feature: Alu sequence cloned from E-313_m74_SZ (see Figure 3)
tatgacatgattacgccaagctctaatacgactcactatagggaaagctcggtaccacgcatgctgcagacgcgttacgtatcggatccagaattcgtgattgcctgtactcccagcagtttgggaggctgagacaggtggaacacttgaggccaggagtttgcaaccagcctggccaacatggtgaaaccctatctctaccacaaaaaaaaaaaaaaaaaaaaaaaaaaaaaaattagcctggcatggtggtgcgtgcctgtaatcccagctactcaggaggctgaggcacgagaatcgcttgaacccggtgggcaagggttgcagcgatccgagattgtgcaaacaccctccaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccc

SEQ ID NO: 70 - DNA, 579 bp, Homo sapiens
  misc_feature: Alu sequence cloned from E-315_m74_SZ (see Figure 3)
tgattacgccaagctctaatacgactcactatagggaaagctcggtaccacgcatgctgcagacgcgttacgtatcggatccagaattcgtgattggagggtgtttgcacaatctcggctcactgcaacttctgcctcctgggttcacactgttctcctgcctaagcctcccaagtagctgggactacaggcgcgtgccaccatgcccggctaattttttgtatttttagtagagaaggggtttcaccgtgttagccaggatggtctcgatctcctgatattgtgatccacccgcctcggcctctcaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggccgcaccgatcgcccttcccaacagttgcgc

SEQ ID NO: 71 - DNA, 563 bp, Homo sapiens
  misc_feature: Alu sequence cloned from E-314_m74_SZ (see Figure 3)
attacgccaagctctaatacgactcactatagggaaagctcggtaccacgcatgctgcagacgcgttacgtatcggatccagaattcgtgattggagggtgtttgcacaatctcggctcactgcaacttctgcctcctgggttcacactgttctcctgcctaagcctcccaagtagctgggactacaggcgcgtgccaccatgcccggctaattttttgtatttttagtagagaaggggtttcaccgtgttagccaggatggtctcgatctcctgatattgtgatccacccgcctcggcctctcaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggccgcaccgatcgccctt

SEQ ID NO: 72 - DNA, 573 bp, Homo sapiens
  misc_feature: Alu sequence cloned from E-319_m74_SZ (see Figure 3)
tatgaccatgattacgccaagctctaataccgactcactatagggaaacgctcggtaccacgcatgctgcagacgcgttacgtatcggatccagaattcgtgattgcctgtactcccagcagtttgggaggccgaggtgggtggatcacctgaggtcaggagttcgagaccagcctggccaacgtagtgaaaaccccatctctactaaaaatacaaaaaaacttagccaggggtggtggtgggcacctataatcccagctacttaggaggctgaggctggagaatcgtttgaacctgggagggagaggttgcagtgagctgagattgtgcaaacaccctccaatctgaattcgtcgacaagcttctcgagcctaggctagctctagaccacacgtgtgggggcccgagctcgcggccgctgtattctatagtgtcacctaaatggccgcacaattcactgggccgtcgttttacaacgtcgtgactgggaaaaccctggcgttacccaacttaatcgccttgcagcacatccccctttcgccagctggcgtaataacgaagaggccgcaccga

SEQ ID NO: 73 - DNA, 650 bp, Homo sapiens
  misc_feature: Alu sequence cloned from E-320_m74_SZ (see Figure 3)
atgattacgccaagctctaatacgactcactatagggaaagctcggtaccacgcatgctgcagacgcgttacgtatcggatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcc

SEQ ID NO: 74 - DNA, 600 bp, Homo sapiens
  misc_feature: Alu sequence cloned from E-321_m74_SZ (see Figure 3)
tatgaccatgattacgccaagctctaatacgactcactatagggaaagctcggtaccacgcatgctgcagacgcgttacgtatcggatccagaattcgtgattggagggtgtttgcacaatctcggctcactgcaacttctgcctcctgggttcacactgttctcctgcctaagcctcccaagtagctgggactacaggcgcgtgccaccatgcccggctaattttttgtatttttagtagagaaggggtttcaccgtgttagccaggatggtctcgatctcctgatattgtgatccacccgcctcggcctctcaaactgctgggagtacaggcaatctgaattcgtcgacaagcttctcgagcctaggctagctctagaccacacgtgtgggggcccgagctcgcggccgctgtattctatagtgtcacctaaatggccgcacaattcactgggccgtcgttttacaacgtcgtgactgggaaaaccctggcgttacccaacttaatcgccttgcagcacatccccctttcgccagctggcgtaatagcgaagaggcccgcacccgatcgcccttcccaacagttgcgcagcctgaatg

SEQ ID NO: 75 - DNA, 600 bp, Homo sapiens
  misc_feature: Alu sequence cloned from E-322_m74_SZ (see Figure 3)
acgtacgctctaatacgactcactatagggaaagctcggtaccacgcatgctgcagacgcgttacgtatcggatccagaattcgtgattggagggtgtttgcacaatcttggctcactgtaacctctgcctcctgggttcaagtaattctcctgtctcagcctcctgagtagctaggattactggtgcccgccaccatgcccggcgaatttttgtatttttagtagagatggggtttcactatgttgcccagggtggtctcaaactcctgacctcaagtgatccacctgcttcagcttcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgcttgcagcacatcccccctttcgccagctggcgtaatagcgaagaggcccgcacccgatcgccccttcccaacagttgcgcagcctgaatggcgaatggaaatt

SEQ ID NO: 76 - DNA, 407 bp, Homo sapiens
  misc_feature: Alu sequence cloned from E-323_m74_SZ (see Figure 3)
aaacgcaagctctaatacgactcactatagggaaagttcggtaccacgcatgctgcagacgcgttacgtatcggatccagaattcgtgattgcctgtactcccagcacgtttgggaagccgaggtgggaagatcgcttcgaggtcaggagttcaagaccagcctggccaacatggcaaaacctcgtctctactaaaaatacaaaacttagccaggccgtgttggcatcgcacccatagtccctgctaatcaggaggctgaggcttgaacatgggaggtggaggctgcagtgagctgagattgtgcaaacaccctccaatctgaattcgtcgacaagcttctcgagcctaggctagctctagaccacacgtgtgggggcccgagctcgcggccgctgtattctatagt

SEQ ID NO: 77 - DNA, 600 bp, Homo sapiens
  misc_feature: Alu sequence cloned from E-324_m74_SZ (see Figure 3)
gttaagatctaatacgactcactatagggaaagctcggtaccacgcatgctgcagacgcgttacgtatcggatccagaattcgtgattggagggtgtttgcacaatctcagctcactgcaacctccacctctacgactcaagtgattatcccacctcaacctcccaagtagcagggactgaaggtgtgctttgccacgcccagctaattttttgtattttttgtagagacggattttcaccatgtagcccaggctggtctcaaactcctgagcttaagcgatccaccttcctggacctcccaaactgctgggagtacaggcaatctgaattcgtcgacaagcttctcgagcctaggctagctctagaccacacgtgtgggggcccgagctcgcggccgctgtattctatagtgtcacctaaatgggccgcacaattcactggccgtcgttttacaacgtcgtgactgggaaaaccctggcgttacccaacttaatcgccttgcagcacatccccctttcgccagctggcgtaatagcgaagaggccgcaccgatcgcccttcccacagttgcgcagcctgaatggcgaatggaaatttaa

SEQ ID NO: 78 - DNA, 501 bp, Homo sapiens
  misc_feature: Alu sequence cloned from E-325_m74_SZ (see Figure 3)
cagctatgaccatgattacgccaagctctaatacgactcactatagggaaagctcggtaccacgcatgctgcagacgcgttacgtatcggatccagaattcgtgatttgccttgtactcccagcagtttgggaggctgaggcaggtgaatcacctgaggtcaggagttcatgaccagcctggccaacatggtgaaaccccgcctctactaaaaatataaaaattagcctgtcatggtagtgctcatctgtaatcccagctgctcaggaagctgaggcagaatttgcttgaacctgggaggcagatgttgcagttagtcaagattgtgcaaacaccctccaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaacctggcgttac

SEQ ID NO: 79 - DNA, 600 bp, Homo sapiens
  misc_feature: Alu sequence cloned from E-149_m48_SZ (see Figure 3)
acgcttccaaggattcaacaagctctaatacgactcactatagggaaagctcggtaccacgcatgctgcagacgcgttacgtatcggatccagaattcgtgattagggtgtttgcacaatctcggctcattgtaacctctgcctcccaggttgcagtgattctcctgtctcagcctcccaagtagctggcattacaggttcccaccactacacccaactaatttttgtatttttagcagaaatggggtttccccatgttgacctggctggtctcgaactcctgaccttgtgatctgcccgccttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcga

SEQ ID NO: 80 - DNA, 480 bp, Homo sapiens
  misc_feature: Alu sequence cloned from E-302_m57_Ctrl (see Figure 3)
gattacgccaagctctaatactactcactatagggaaagctcggtaccacgcatgctgcagacgcgttacgtatcggatccagaattcgtgattggagggtgtttgcacaatctcagctcaccgaaacctccgcctcacaggttcaagtgattcctctgcctcagccttctgagtagctaggacgacaagcatttgccatgatacctggctaattttgtatttttagtagagaccaggattcttcatgttgataaggtggttcttgaactcctgacctcagatgatccacctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaacctg

SEQ ID NO: 81 - DNA, 610 bp, Homo sapiens
  misc_feature: Alu sequence cloned from E-119m57Ctrl (see Figure 3)
cagctatgaccatgattacgccaagctctaatacgactcactatagggaaagctcggtaccacgcatgctgcagacgcgttacgtatcggatccagaattcgtgattgcctgtactcccagcagtttgggaggcagaggcaggtggatcacctgaggtcgggagttcgagaaccgcctgaccaacatggagaaaccccgtctctgctaaaaatacaaaattagctaggtatggtggtacttgcccgtaatcccagctattcagaaggctgaggcaggagagtcacttgaacccaggagtcagaggttgcagtcagctgagattgtgcaaacaccctccaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgg

SEQ ID NO: 82 - DNA, 470 bp, Homo sapiens
  misc_feature: Alu sequence cloned from E-120m57Ctrl (see Figure 3)
aatagctatgcccatgattacgccaagctctaatacgactcactatagggtatgctcggagctaggcatgctgcagacgcgttacgcattacgatccagaatccagagattggaggtggctggcgtaatatcggtttagtgggacctgtgcctccgggttccaggtgttgctagtgtttgaacctcctgagcatcattggataacagtagcctctcaccatgctcatcttgtgcttgtattggtggcagcggtccaccatgccggttatgctgaactcggactcatcaccttaaattaaccacctgcctcagactccgaaactgctggtagtacaggcaatctgcattcgtctgcattcttctacagcctaggctagctatagaccacacttgaccacggcccgagctcccggccgcttggattctatagtgtcatataaaggcccgaacaattcactgcaccgtagttt

SEQ ID NO: 83 - DNA, 620 bp, Homo sapiens
  misc_feature: Alu sequence cloned from E-166m50Ctrl (see Figure 3)
aacagctatgaccatgattacgccaagctctaatacgactcactatagggaaagctcggtaccacgcatgctgcagacgcgttacgtatcggatccagaattcgtgattggagggtgtttgcacaatctcggcccactgcaacctccgcctcccgggtgcaagcagttctcctacctcagcctcctgagtagctaggattacaggcacacctggctaattttgtggttttagtagagacggcgtttcaccatgttggctaggctggtctcgaactcctcacctcaaatgatccacctgcctcagcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgccttcccaacagttgcgcagcctgaatggcgaatggaaattgtaagccgttaata

SEQ ID NO: 84 - DNA, 600 bp, Homo sapiens
  misc_feature: Alu sequence cloned from E-167m50Ctrl (see Figure 3)
actttatgacatgattacgccaagctctaatacgactcactatagggaaagctcggtaccacgcatgctgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgccttcccaacagttgcgcagcctgaatg

SEQ ID NO: 85 - DNA, 480 bp, Homo sapiens
  misc_feature: Alu sequence cloned from E-169m50Ctrl (see Figure 3)
aagcttgaccatgattacgccaagctctaatacgactcactatagggaaagctcggtaccacgcatgctgcagacgcgttacgtatcggatccagaattcgtgattgcctgtactcccagcagtttgggaggctgaagtgggttgattacccgaggtcaggagttccagaccaggttgaccaacatggagaaaccctgtctctactaaaaatacataattagccaggtgtattggagcgtgcctgtattcccagctacttgggaggccgaggcaggagaatctgctggaacccacgatggcggaggttgtggagagctgagattgtgcaaacaccctccaatctgaattcgtctacaagcttctcgagcctaggttagctctagaccacacgtgtgggggcccgagctcgcggacgctgtattctatagtgtcacctaaatggccgcacaattcactggccgacgttttacaacgtggtg

SEQ ID NO: 86 - DNA, 610 bp, Homo sapiens
  misc_feature: Alu sequence cloned from E-270m50Ctrl (see Figure 3)
ctcactatagggaaagctcggtaccacgcatgctgcagacgcgttacgtatcggatccagaattcgtgattgcctgtactcccagcagtttgggaggccaaatcagatggatcatctgaggtcaggagttcaagaaccaccttatcaacatgaagaatcctggtctctactaaaaatacaaaattagccaggtatcatggcaaatgcttgtcatcctagctactcagaaggctgaggcagaggaatcacttgaacctgtgaggcggaggtttcggtgagctgagattgtgcaaacaccctccaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgt

SEQ ID NO: 87 - DNA, 601 bp, Homo sapiens
  misc_feature: Alu sequence cloned from E-271m50Ctrl (see Figure 3)
ttgcccatgcttacgccaagctctaatacgactcactatagggaaagctcggtaccacgcatgctgcagacgcgttacgtatcggatccagaattcgtgattggagggtgtttgcacaatctcagctcaccatgacctctgcctcctgggttcaagcgattctctggactcagcctcctgagtagctggaattacagggattcgccaccatgcccagctaattttgtatgtttagtagagacagggtttctccaaattggtcaggctggtctcgaactcccgacctcaggtgatccgcccgccttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcga

SEQ ID NO: 88 - DNA, 601 bp, Homo sapiens
  misc_feature: Alu sequence cloned from E-272m50Ctrl (see Figure 3)
caataccgcttgaccatgattacgccaagctctaatacgactactatagggaaagctcggtaccacgcatgctgcagacgcgttacgtatcggatccagaattcgtgattggagggtgtttgcacaatctcagctcactgcagcctcctccctctgaggtcaagtgatactgctgcctcagcctcctgagtagctgggattacaggcacccaccaccaaccctggccaatttttgtatttttagtagagacagagtttcaccatgctggccaggctggtctcaaactcctgccctcagatgttccacccaccttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttccaacagttgcgcagcctg

SEQ ID NO: 89 - DNA, 479 bp, Homo sapiens
  misc_feature: Alu sequence cloned from E-273m50Ctrl (see Figure 3)
gctcggtaccacgcatgctgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattctttatgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccggcgttttacaacgtcgcgactgggaaaaccctggcgttacccaacttaatcgccttgcagcacatcccc

SEQ ID NO: 90 - DNA, 600 bp, Homo sapiens
  misc_feature: Alu sequence cloned from E-275m50Ctrl (see Figure 3)
accatgattacgccaagctctaatacgactcactatagggaaagctcggtaccacgcatgctgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaat

SEQ ID NO: 91 - DNA, 610 bp, Homo sapiens
  misc_feature: Alu sequence cloned from E-279m50Ctrl (see Figure 3)
aagaccatgataacgccaagctctaatacgactcactatagggaaagctcggtaccacgcatgctgcagacgcgttacgtatcggatccagaattcgtgattggagggtgtttgcacaatctcagctcactgcagcctcctccctctgaggtcaagtgattctgctgcctcagcctcctgagtagctgggattacaggcacccaccaccaaccctggccaatttttgtatttttagtagagacagagtttcaccatgctggccaggctggtctcaaactcctgccctcagatgttccacccaccttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaat

SEQ ID NO: 92 - DNA, 602 bp, Homo sapiens
  misc_feature: Alu sequence cloned from E-281m50Ctrl (see Figure 3)
aacagctatgaccatgattacgccaagctctaatacgactcactatagggaaagctcggtaccacgcatgctgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaataacgaagaggcccgcaccgatcgcccttcccaacagttgcgcagcctgaa

SEQ ID NO: 93 - DNA, 601 bp, Homo sapiens
  misc_feature: Alu sequence cloned from E-283m56SZ (see Figure 3)
aacagctatgaccatgattacgccaagctctaatacgactcactatagggaaagctcggtaccacgcatgctgcagacgcgttacgtatcggatccagaattcgtgattggagggtgtttgcacaatcttggctcactgtaacctctgcctcttgggttcaagtaattctcctgtctcagcctcctgagtagctaggattactggtgcccgccaccatgcccggcaaatttttgtatttttagtagagatggggtttcactatgttgcccagggtggtctcaaactcctgacctcaagtgatccacctgcttcagcttcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgccttcccaacagttgcgcagcctga

SEQ ID NO: 94 - DNA, 620 bp, Homo sapiens
  misc_feature: Alu sequence cloned from E-284m56SZ (see Figure 3)
agctatgaccatgattacgccaagctctaatacgactcactatagggaaagctcggtaccacgcatgctgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgccttcccaacagttgcgcagcctgaatggcgaatggaaattgtaagcg

SEQ ID NO: 95 - DNA, 600 bp, Homo sapiens
  misc_feature: Alu sequence cloned from E-61m34BD (see Figure 3)
ttaaacagctatgaccatgattacgccaagctctaatacgactcactatagggaaagctcggtaccacgcatgctgcagacgcgttacgtatcggatccagaattcgtgattggagggtgtttgcacaatctcggttcactgcaacttctgcctcccaggttcaagcaattatctgcctcagcctcccgagtagctgggattacaggtgcccgccaccacactcagctaattttcgtatttttagtagagacggtttcaccatcttggctaggctggtcttgagctcctgactgcgtgatccacccgccttggccccccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctga

SEQ ID NO: 96 - DNA, 627 bp, Homo sapiens
  misc_feature: Alu sequence cloned from E-62m34BD (see Figure 3)
cttgaccatgattacgccaagctctaatacgactcactatagggaaagctcggtaccacgcatgctgcagacgcgttacgtatcggatccagaattcgtgattggagggtgtttgcacaatcttggctcactgtaacctctgcctcctgggttcaagtaattctcctgtctcagcctcctgagtagctaggattactggtgcccgccaccatgcccggcaaatttttgtatttttagtagagatggggtttcactatgttgcccagggtggtctcaaactcctgacctcaagtgatccacctgcttcagcttcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatatt

SEQ ID NO: 97 - DNA, 610 bp, Homo sapiens
  misc_feature: Alu sequence cloned from E-63m34BD (see Figure 3)
aacagctatgaccatgattacgccaagctctaatacgactcactatagggaaagctcggtaccacgcatgctgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgacttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgccttcccaacagttgcgcagcctgaatggcgaatg

SEQ ID NO: 98 - DNA, 577 bp, Homo sapiens
  misc_feature: Alu sequence cloned from E-66m39MD (see Figure 3)
tatgaccatgattacgccaagctctaatacgactcactatagggaaagctcggtaccacgcatgctgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaatctgaattcgtcgacaagcttctcgagcctaggctagctatagaccacacgtgtgggggcccgagctcgcggccgctgtattctatagtgtcacctaaatggccgcacaattcactggccgtcgttttacaacgtcgtgactgggaaaaccctggcgttacccaacttaatcgcttgcagcacatcccctttcgccagctggcgtaatagcgaagaggcccgcaccgatcgcccttcccaa

SEQ ID NO: 99 - DNA, 680 bp, Homo sapiens
  misc_feature: Alu sequence cloned from E-68m39MD (see Figure 3)
cagctatgaccatgattacgccaagctctaatacgactcactatagggaaagctcggtaccacgcatgctgcagacgcgttacgtatcggatccagaattcgtgattggagggtgtttgcacaatctcagctcaccgaaacctccgcctcacaggttcaagtgattcctctgcctcagccttctgagtagctaggatgacaagcatttgccatgatacctggctaattttgtatttttagtagaggccaggattcttcatgttgataaggtggttcttgaactcctgacctcagatgatccatctgatttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgccttcccaacagttgcgcagcctgaatggcgaatggaaattgtaagcgttaatattttgttaaaattcgcgttaaatttttgttaaatcaactcattttttaaccaa

SEQ ID NO: 100 - DNA, 581 bp, Homo sapiens
  misc_feature: Alu sequence cloned from E-71m39MD (see Figure 3)
aagattgaccatgattacgccaagctctaatacgactcactatagggaaagctcggtaccacgcatgctgcagacgcgttacgtatcggatccagaattcgtgattggagggtgtttgcacaatctcagctcactgcaaccttcacctcccaggttcaagcgattctcatgcctcagccttccgaatagttgagattacaggctcgtgccaccacacccagctaattttttgtatttttagtagagatggggtttcaccatgttggccaggctggtcttgagctcctgacctcaagtaatctgcccacctcagcctccaaaactgctgggagtacaggcaatctgaattcgtcgacaagcttctcgagcctaggctagctctagaccacacgtgtgggggcccgagctcgcggccgatgtattctatagtgtcacctaaatggccgcacaattcactggccgtcgttttacaacgtcgagactgggaaaaccctggcgttacccaacttaatcgccttgcagcacatccccctttcgccagctggcgtaatagcgaagaggcccgcaccgatcgacctttc

SEQ ID NO: 101 - DNA, 600 bp, Homo sapiens
  misc_feature: Alu sequence cloned from E-72m43BD (see Figure 3)
taaacacgttgaccatgattacgccaagctctaatacgactcactatagggaaagctcggtaccacgcatgctgcagacgcgttacgtatcggatccagaattcgtgattggagggtgtttgcacaatctcggctcactgcaacatccgcctcccgagtagctgggaccacaggtgtgcaccacctttccgggctaatttttgtatttttagtagagacagggttttgccatgttggtcaggctggtcttgaactcctgacctcaggtgatttgcccacctcagcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaata

SEQ ID NO: 102 - DNA, 622 bp, Homo sapiens
  misc_feature: Alu sequence cloned from E-74m43BD (see Figure 3)
aaacagctatgaccatgattacgccaagctctaatacgactcactatagggaaagctcggtaccacgcatgctgcagacgcgttacgtatcggatccagaattcgtgattggagggtgtttgcacaatctcagctcattgcgagctccacctcccaggttcaagcaattctcctacctcagcaactcctgagtagctgagactacaggtgtgtgccactatgcctggctaactttttttgtatttttagtagagacagggtttcaccatgttggccaggctagtctcgaacacctgacctcagatgatccacctgcctcggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagctgaatggcgaatggaaat

SEQ ID NO: 103 - DNA, 670 bp, Homo sapiens
  misc_feature: Alu sequence cloned from E-75m43BD (see Figure 3)
cagctatgaccatgattacgccaagctctaatacgactcactatagggaaagctcggtaccacgcatgctgcagacgcgttacgtatcggatccagaattcgtgattggagggtgtttgcacaatcttggttcactacaacctccaatctccaggttcaaggattctcctgcctcagactcctgagtagctgggattacaggcatccaccaacatgcctggctaatttttttatttttagcagagacggggttttgccatattggccatgctggtctcaaactcctgacctcatgtgatccacccgccttggcctcccaaactgctgggagtacaggcaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcat

SEQ ID NO: 104 - DNA, 570 bp, Homo sapiens
  misc_feature: Alu sequence cloned from E-77m43BD (see Figure 3)
cagctaacagctatgacctgattacgccaagctctaatacgactcactatagggaaagctcggtaccacgcatgctgcagacgcgttacgtatcggatccagaattcgtgattgcctgtactcccagcagtttcggaggttgaggcgggtggattacctgaggtcaggagtttaagatcagcctggccaacctgatgaaaccccatctctactaaaaatacaaaaaattagcctggtgtgttggtgggcatctgtaatcccagctactcgggaggctgaggcaggataatcacttgaacctgggaggtggtggttgcagtgagctgagattgtgcaaacaccctccaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcc

SEQ ID NO: 105 - DNA, 601 bp, Homo sapiens
  misc_feature: Alu sequence cloned from E-78m43BD (see Figure 3)
acagctatgaccatgattacgccaagctctaatacgactcactatagggaaagctcggtaccacgcatgctgcagacgcgttacgtatcggatccagaattcgtgattggagggtgtttgcacaatctcggctcaatgcaacctcagcctcctgggttcaagcaattctcctgtctcagcctcccgagtagctgggattacaggcacatgccaccatgcccaactaatttttgtatttttagtagagacagggttttgccatgttggccaggctggtctcaaactcctgacctcaggtggtccaccggcctcagcctcccaaactgctgggagtacaggccaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgccttccaacagttgcgcagcctgaa

SEQ ID NO: 106 - DNA, 520 bp, Homo sapiens
  misc_feature: Alu sequence cloned from E-79m43BD (see Figure 3)
aacagctatgaccatgattacgccaagctctaatacgactcactatagggaaagctcggtaccacgcatgctgcagacgcgttacgtatcggatccagaattcgtgattggagggtgtttgcacaatctcagctcactgcaacctccgtttcccaggtgcaaccgattctcctgcctcagacctctgaagcggctgggactacaggtgcctgccacctcacccggctaatttttgtatttttagtaagagatggggtttcaccacattggccggggtggtctcaaactcctgacctcaagtgatccttccatcttggcctcccaaactgctgggagtacaggcaatctgaattcgtcgacaagcttctcgagcctaggctagctctataccacacgtgtgggggcccgagctccgcggccgctgtattctatagtgttacctaaatggccggacaattcactggccgtcggtttacaacgtcaggactgggaaaaccctggcgttacccaacttaatgcc

SEQ ID NO: 107 - DNA, 591 bp, Homo sapiens
  misc_feature: Alu sequence cloned from E-83m43BD (see Figure 3)
cagctatgaccatgattacgccaagctctaatacgactcactatagggaaagctcggtaccacgcatgctgcagacgcgttacgtatcggatccagaattcgtgattggagggtgtttgcacaatctcggctcaatgcaacctcagcctcctgggttcaagcaattctcctgtctcagcctcccgagtagctgggattacaggcacatgccaccatgcccaactaatttttgtatttttagtagagacagggttttgccatgttggccaggctggtctcaaactcctgacctcaggtggtccaccggcctcagcctcccaaactgctgggagtacaggccaatctgaattcgtcgacaagcttctcgagcctaggctagctctagaccacacgtgtgggggcccgagctcgcggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgccttccaacagttgcg

SEQ ID NO: 108 - DNA, 191 bp, Homo sapiens
  misc_feature: Alu sequence cloned from E-167m50Ctrl (see Figure 3)
cagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctccc

SEQ ID NO: 109 - DNA, 191 bp, Homo sapiens
  misc_feature: Alu sequence cloned from E-271m50Ctrl (see Figure 3)
cagctcaccatgacctctgcctcctgggttcaagcgattctctggactcagcctcctgagtagctggaattacagggattcgccaccatgcccagctaattttgtatgtttagtagagacagggtttctccaaattggtcaggctggtctcgaactcccgacctcaggtgatccgcccgccttggcctccc

SEQ ID NO: 110 - DNA, 192 bp, Homo sapiens
  misc_feature: Alu sequence cloned from E-272m50Ctrl (see Figure 3)
cagctcactgcagcctcctccctctgaggtcaagtgatactgctgcctcagcctcctgagtagctgggattacaggcacccaccaccaaccctggccaatttttgtatttttagtagagacagagtttcaccatgctggccaggctggtctcaaactcctgccctcagatgttccacccaccttggcctccc

SEQ ID NO: 111 - DNA, 191 bp, Homo sapiens
  misc_feature: Alu sequence cloned from E-273m50Ctrl (see Figure 3)
cagctcaccgaaacctccgcctcacaggttcaagtgattcctctgcctcagccttctgagtagctaggatgacaagcatttgccatgatacctggctaattttgtatttttagtagagaccaggattctttatgttgataaggtggttcttgaactcctgacctcagatgatccatctgatttggcctccc

SEQ ID NO: 112 - DNA, 191 bp, Homo sapiens; identical to SEQ ID NO: 108
  misc_feature: Alu sequence cloned from E-275m50Ctrl (see Figure 3)

SEQ ID NO: 113 - DNA, 192 bp, Homo sapiens
  misc_feature: Alu sequence cloned from E-279m50Ctrl (see Figure 3)
cagctcactgcagcctcctccctctgaggtcaagtgattctgctgcctcagcctcctgagtagctgggattacaggcacccaccaccaaccctggccaatttttgtatttttagtagagacagagtttcaccatgctggccaggctggtctcaaactcctgccctcagatgttccacccaccttggcctccc

SEQ ID NO: 114 - DNA, 191 bp, Homo sapiens; identical to SEQ ID NO: 108
  misc_feature: Alu sequence cloned from E-281m50Ctrl (see Figure 3)

SEQ ID NO: 115 - DNA, 192 bp, Homo sapiens
  misc_feature: Alu sequence cloned from E-283m56SZ (see Figure 3)
tggctcactgtaacctctgcctcttgggttcaagtaattctcctgtctcagcctcctgagtagctaggattactggtgcccgccaccatgcccggcaaatttttgtatttttagtagagatggggtttcactatgttgcccagggtggtctcaaactcctgacctcaagtgatccacctgcttcagcttccc

SEQ ID NO: 116 - DNA, 191 bp, Homo sapiens; identical to SEQ ID NO: 108
  misc_feature: Alu sequence cloned from E-284m56SZ (see Figure 3)

SEQ ID NO: 117 - DNA, 187 bp, Homo sapiens
  misc_feature: Alu sequence cloned from E-61m34BD (see Figure 3)
cggttcactgcaacttctgcctcccaggttcaagcaattatctgcctcagcctcccgagtagctgggattacaggtgcccgccaccacactcagctaattttcgtatttttagtagagacggtttcaccatcttggctaggctggtcttgagctcctgactgcgtgatccacccgccttggcccccc

SEQ ID NO: 118 - DNA, 192 bp, Homo sapiens
  misc_feature: Alu sequence cloned from E-62m34BD (see Figure 3)
tggctcactgtaacctctgcctcctgggttcaagtaattctcctgtctcagcctcctgagtagctaggattactggtgcccgccaccatgcccggcaaatttttgtatttttagtagagatggggtttcactatgttgcccagggtggtctcaaactcctgacctcaagtgatccacctgcttcagcttccc

SEQ ID NO: 119 - DNA, 191 bp, Homo sapiens
  misc_feature: Alu sequence cloned from E-63m34BD (see Figure 3)
cagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgacttggcctccc

SEQ ID NO: 120 - DNA, 191 bp, Homo sapiens; identical to SEQ ID NO: 108
  misc_feature: Alu sequence cloned from E-66m39MD (see Figure 3)

SEQ ID NO: 121 - DNA, 191 bp, Homo sapiens
  misc_feature: Alu sequence cloned from E-68m39MD (see Figure 3)
cagctcaccgaaacctccgcctcacaggttcaagtgattcctctgcctcagccttctgagtagctaggatgacaagcatttgccatgatacctggctaattttgtatttttagtagaggccaggattcttcatgttgataaggtggttcttgaactcctgacctcagatgatccatctgatttggcctccc

SEQ ID NO: 122 - DNA, 193 bp, Homo sapiens
  misc_feature: Alu sequence cloned from E-71m39MD (see Figure 3)
cagctcactgcaaccttcacctcccaggttcaagcgattctcatgcctcagccttccgaatagttgagattacaggctcgtgccaccacacccagctaattttttgtatttttagtagagatggggtttcaccatgttggccaggctggtcttgagctcctgacctcaagtaatctgcccacctcagcctcca

SEQ ID NO: 123 - DNA, 160 bp, Homo sapiens
  misc_feature: Alu sequence cloned from E-72m43BD (see Figure 3)
cggctcactgcaacatccgcctcccgagtagctgggaccacaggtgtgcaccacctttccgggctaatttttgtatttttagtagagacagggttttgccatgttggtcaggctggtcttgaactcctgacctcaggtgatttgcccacctcagcctccc

SEQ ID NO: 124 - DNA, 197 bp, Homo sapiens
  misc_feature: Alu sequence cloned from E-74m43BD (see Figure 3)
cagctcattgcgagctccacctcccaggttcaagcaattctcctacctcagcaactcctgagtagctgagactacaggtgtgtgccactatgcctggctaactttttttgtatttttagtagagacagggtttcaccatgttggccaggctagtctcgaacacctgacctcagatgatccacctgcctcggcctccc

SEQ ID NO: 125 - DNA, 191 bp, Homo sapiens
  misc_feature: Alu sequence cloned from E-75m43BD (see Figure 3)
tggttcactacaacctccaatctccaggttcaaggattctcctgcctcagactcctgagtagctgggattacaggcatccaccaacatgcctggctaatttttttatttttagcagagacggggttttgccatattggccatgctggtctcaaactcctgacctcatgtgatccacccgccttggcctccc

SEQ ID NO: 126 - DNA, 192 bp, Homo sapiens
  misc_feature: Alu sequence cloned from E-78m43BD (see Figure 3)
cggctcaatgcaacctcagcctcctgggttcaagcaattctcctgtctcagcctcccgagtagctgggattacaggcacatgccaccatgcccaactaatttttgtatttttagtagagacagggttttgccatgttggccaggctggtctcaaactcctgacctcaggtggtccaccggcctcagcctccc

SEQ ID NO: 127 - DNA, 194 bp, Homo sapiens
  misc_feature: Alu sequence cloned from E-79m43BD (see Figure 3)
cagctcactgcaacctccgtttcccaggtgcaaccgattctcctgcctcagacctctgaagcggctgggactacaggtgcctgccacctcacccggctaatttttgtatttttagtaagagatggggtttcaccacattggccggggtggtctcaaactcctgacctcaagtgatccttccatcttggcctccc

SEQ ID NO: 128 - DNA, 192 bp, Homo sapiens; identical to SEQ ID NO: 126
  misc_feature: Alu sequence cloned from E-83m43BD (see Figure 3)

SEQ ID NO: 129 - DNA, 470 bp, Homo sapiens; identical to SEQ ID NO: 82
  misc_feature: Alu sequence cloned from E-120m57Ctrl (see Figure 3)

SEQ ID NO: 130 - DNA, 470 bp, Homo sapiens
  misc_feature: Alu sequence cloned from RevE-120m57Ctrl (see Figure 3)
aaactacggtgcagtgaattgttcgggcctttatatgacactatagaatccaagcggccgggagctcgggccgtggtcaagtgtggtctatagctagcctaggctgtagaagaatgcagacgaatgcagattgcctgtactaccagcagtttcggagtctgaggcaggtggttaatttaaggtgatgagtccgagttcagcataaccggcatggtggaccgctgccaccaatacaagcacaagatgagcatggtgagaggctactgttatccaatgatgctcaggaggttcaaacactagcaacacctggaacccggaggcacaggtcccactaaaccgatattacgccagccacctccaatctctggattctggatcgtaatgcgtaacgcgtctgcagcatgcctagctccgagcataccctatagtgagtcgtattagagcttggcgtaatcatgggcatagctatt

SEQ ID NO: 131 - DNA, 191 bp, Homo sapiens
  misc_feature: Alu sequence cloned from RevE-119m57Ctrl (see Figure 3)
cagctgactgcaacctctgactcctgggttcaagtgactctcctgcctcagccttctgaatagctgggattacgggcaagtaccaccatacctagctaattttgtatttttagcagagacggggtttctccatgttggtcaggcggttctcgaactcccgacctcaggtgatccacctgcctctgcctccc

SEQ ID NO: 132 - DNA, 191 bp, Homo sapiens; identical to SEQ ID NO: 108
  misc_feature: Alu sequence cloned from RevE-270m50Ctrl (see Figure 3)

SEQ ID NO: 133 - DNA, 193 bp, Homo sapiens
  misc_feature: Alu sequence cloned from RevE-169m50Ctrl (see Figure 3)
cagctctccacaacctccgccatcgtgggttccagcagattctcctgcctcggcctcccaagtagctgggaatacaggcacgctccaatacacctggctaattatgtatttttagtagagacagggtttctccatgttggtcaacctggtctggaactcctgacctcgggtaatcaacccacttcagcctccc

SEQ ID NO: 134 - DNA, 193 bp, Homo sapiens
  misc_feature: Alu sequence cloned from RevE-77m43BD (see Figure 3)
cagctcactgcaaccaccacctcccaggttcaagtgattatcctgcctcagcctcccgagtagctgggattacagatgcccaccaacacaccaggctaattttttgtatttttagtagagatggggtttcatcaggttggccaggctgatcttaaactcctgacctcaggtaatccacccgcctcaacctccg

SEQ ID NO: 135 - DNA, 191 bp, Homo sapiens; identical to SEQ ID NO: 108
  misc_feature: Alu sequence cloned from PK1601mM-13_m37-7+++ (see Figure 3)

SEQ ID NO: 136 - DNA, 191 bp, Homo sapiens
  misc_feature: Alu sequence cloned from PK1601mM-11_m37-5+++ (see Figure 3)
cagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggccttcc

SEQ ID NO: 137 - DNA, 306 bp, Homo sapiens
  misc_feature: Alu sequence cloned from PK1601_mM-1_m57-6 (see Figure 3)
cagctcactgcaggctccgcctcccgggttcacgccattctcctgcctcagcctcccgagtagctgggactacaggcgcccaccaccatgcccagctaatttttgtatttttagcagagacggggtttcaccatgttggccaggatggtctccaaactcctgacctcctgagacacctgtgtcggggtcccaaactgtgggagtacaggcaactctgaatttttggacaagactcttcgagcctatgctactatctacaccacaccgcgtgggggccccagctcgcggccgctgtattatataata

SEQ ID NO: 138 - DNA, 187 bp, Homo sapiens
  misc_feature: Alu sequence cloned from PK1601mM-60+++ (see Figure 3)
cagctcaatgcaacctacacctcctgggttcaagtgattctcacgcctcagcctcctaagtaactgggattacaggggcgcaccaccacacctggctaattttttgtatttttagcagagatgggccatgttggccaggctggtcttgaactcctgacctcaagtgatccacctgcctcggcctccc

SEQ ID NO: 139 - DNA, 191 bp, Homo sapiens; identical to SEQ ID NO: 108
  misc_feature: Alu sequence cloned from PK1601MM-59+++ (see Figure 3)

SEQ ID NO: 140 - DNA, 191 bp, Homo sapiens; identical to SEQ ID NO: 108
  misc_feature: Alu sequence cloned from PK1601mM-58+++ (see Figure 3)

SEQ ID NO: 141 - DNA, 418 bp, Homo sapiens
  misc_feature: Alu sequence cloned from PK1601mM-57+++ (see Figure 3)
atctatgacatgattgccccgattctccaagctctaattctactgaatgttcggaacgctccatccacgcatgccgtaaacgctttactcctcggttccagaatgcgggattgcctgtacttccatcagttagggaggccaaatcctacggatcatatgaggctatgagaccaagacccaccttatcaacatgaagaatcctggtctctactaaaaatacaatattagccaggtttcatggtatatgcttgtaatcctagctactcacaaggctgaggcagaggaattacttgaacctgtgaggcggaggtttcggtgagctgagattgtccaaacaccctccaatctgaattcgttgacaagcttttcgagcctaggctagctctagaccacacgtgtgggggcccgagctcgcggt

SEQ ID NO: 142 - DNA, 380 bp, Homo sapiens
  misc_feature: Alu sequence cloned from PK1601mM-55+++ (see Figure 3)
acgttgcctgttcgcagttatcgctacttgggaagtcgtcccatctgagccgtcgatcgatccagaatcggattggaggtgttgccaacattgagtcactgcagctttgacctcctgagtgcatgtggcttattccacctcaacctcctgaggagttgggaccaccagtgttcaacaccacatcaggctaatttaatattttgtagaaatgaagacttactattatgtccaggctagtattaaaatactggggttaagcaagactccccccttgttgttcccaaatgctggggggacaacaggtattgatttttcgacaagcttcttcgagcctccgatggttctatacaccacacgtggggcccgagctctcgccgctg

SEQ ID NO: 143 - DNA, 191 bp, Homo sapiens; identical to SEQ ID NO: 108
  misc_feature: Alu sequence cloned from Pk1601mM-54+++ (see Figure 3)

SEQ ID NO: 144 - DNA, 191 bp, Homo sapiens
  misc_feature: Alu sequence cloned from pk1601mM-53+++ (see Figure 3)
cagctcaccgaaacctgcgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctccc

SEQ ID NO: 145 - DNA, 192 bp, Homo sapiens
  misc_feature: Alu sequence cloned from pk1601mM-52+++ (see Figure 3)
cagctcactgcaacctccgcctcctggattcaagcgattttcccgccttagcctcctgagtaactgggactagaggcaggtaccaccacgcccagctaatttttgtatttttagtagagacgaggtttcaccatgtgggccaggctggtcttaaactcctgacctcaagtgatttgcccaactcagcctccc

SEQ ID NO: 146 - DNA, 192 bp, Homo sapiens; identical to SEQ ID NO: 145
  misc_feature: Alu sequence cloned from pk1601mM-51+++ (see Figure 3)

SEQ ID NO: 147 - DNA, 191 bp, Homo sapiens; identical to SEQ ID NO: 108
  misc_feature: Alu sequence cloned from pk1601mM-50 (see Figure 3)

SEQ ID NO: 148 - DNA, 192 bp, Homo sapiens
  misc_feature: Alu sequence cloned from pk1601mM-49 (see Figure 3)
gactcattgcaacctctgcctcctgggtttaagccgttctcatgcctcagcctcccgacgtagctgggattataggcatgcgccaccacccccagctaatttttgtattatcagtagagatggggcttcgccatgctggccaggctggtcttgaactcctgacctcaagcaatccgcccaactcggcctccc

SEQ ID NO: 149 - DNA, 191 bp, Homo sapiens
  misc_feature: Alu sequence cloned from pk1601mM-47 (see Figure 3)
cagctcaccgaaacctccgcctcacgggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctccc

SEQ ID NO: 150 - DNA, 191 bp, Homo sapiens; identical to SEQ ID NO: 108
  misc_feature: Alu sequence cloned from pk1601mM-48 (see Figure 3)

SEQ ID NO: 151 - DNA, 190 bp, Homo sapiens
  misc_feature: Alu sequence cloned from pk1601mM-44 (see Figure 3)
cagctcaccgaaacctccgcctcacaggttcaagtgattcctctgcctcagccttctgagtagctaggatgacaagcatttgccatgatacctggctaattttgtattttagtagagaccaggattcttcatgttgataaggtggttcttgaactcctgacctcagatgatccatctgatttggcctccc

SEQ ID NO: 152 - DNA, 191 bp, Homo sapiens
  misc_feature: Alu sequence cloned from pk1601mM-42 (see Figure 3)
cagctcaccgaaacctccgcctcacaggttcaagtgattcctctgcctcagccttctgagtagctaggatgacaagcatttgccatgatacctggctaattttgtatgtttagtagagaccaggattcttcatgttgataaggtggttcttgaactcctgacctcagatgatccatctgatttggcctccc

SEQ ID NO: 153 - DNA, 320 bp, Homo sapiens
  misc_feature: Alu sequence cloned from pk1601mM-37+++ (see Figure 3)
gacaggtatgaccatgattacgccagctctaatacgactcactatagggaaagctcggtaccacgcatgctgcagacgcgttacgtatgggatccagaattcgtgattggagggtgttttgcacaatctcagctcaccgcaacctttgcctcacgggctcaagtgattctcatgcttgatcctaccaagtagctgggattacaggcacatgccatcatgctgagctaactttggtatttttggtagagacgaggtttcaccatgttggccaggctgtctcaaactcctgacctcagatgatccgtccacctcagcctccc

SEQ ID NO: 154 - DNA, 191 bp, Homo sapiens
  misc_feature: Alu sequence cloned from pk1601mM-35+++ (see Figure 3)
cggctcactgcaagctctgcctcccgggttcatgccattctcctgcctcagcctcccgagtagctgggactgcaggtggccgtcaccacgcccggctaattttttgtatttttagtagagacagggtttcaccatgttagccaggatggtctcgatctcctgacctcgtgatctgcccgcctcagcctccc

SEQ ID NO: 155 - DNA, 188 bp, Homo sapiens
  misc_feature: Alu sequence cloned from pk1601_mM-32+++ (see Figure 3)
cacgtcactgtaatgtccatctcccgggttcaggtgattctcctgccccagcctcctgagtagctgtacaggcgtgcaccaccatgcccgactaatttttgtacttttagtagagattgggtttcaccgtgttggtcaggctggtcttgaactcctgacctcaagtgatctgcctgcctcagcctccc

SEQ ID NO: 156 - DNA, 140 bp, Homo sapiens
  misc_feature: Alu sequence cloned from pk1601_mM-31+++ (see Figure 3)
cagcttactgcaacctttgcttcccagtttcaagtgattctcctgtctcatgctccagagaacccggtactacaggcacacgccaccatgctcggctaataatttatgttcttagaatagagattggttttcaccgattt

SEQ ID NO: 157 - DNA, 190 bp, Homo sapiens
  misc_feature: Alu sequence cloned from pk1601_mM-30+++ (see Figure 3)
tggctcactgcaacctctgccacccggatttaagcaattctcctgcctcagcctcccgagtagctgggattacaggcgcctgccactgctctgagctaatttttgtatttttggtagagacgggatttcaccatcttggccaggctggttttaaactcctgacctcatgatccacccgcctcggccttcc

SEQ ID NO: 158 - DNA, 292 bp, Homo sapiens
  misc_feature: Alu sequence cloned from pk1401_mM-24+++ (see Figure 3)
tggcttactggaaccttcgccttccgggttcaagagattcttctgccttaaccttccgagaggctgggactacaggcatgcgccaccatgcccagctaggttttggatttttaagagagatggggtttccccatgttggccaggatgatctcgatctcttgacctcgtgatctgtccggcttaagacttccaaactggtgggagtacaggcaatctgaattcgtcgacaagcttttctagcctaggctagctctagacacacgtgtgggggcccgagctcgcggccgctgta

SEQ ID NO: 159 - DNA, 192 bp, Homo sapiens
  misc_feature: Alu sequence cloned from pk1401_mM-23+++ (see Figure 3)
cggttcattgcaacctccgcttcctagggtccagtgatcctcctgcctcagtcccccaagtggctgggactacaggcatgtgccaccacatctggctaacttttgtatatttagtagaaacagggtttcaccatgttggccaggctggtctcgaactcctggcctcaagtgatccacccgccttggcctccc

SEQ ID NO: 160 - DNA, 191 bp, Homo sapiens; identical to SEQ ID NO: 108
  misc_feature: Alu sequence cloned from pk1401_mM-22+++ (see Figure 3)

SEQ ID NO: 161 - DNA, 190 bp, Homo sapiens
  misc_feature: Alu sequence cloned from pk1401_mM-21+++ (see Figure 3)
tggctcactgcaacctctgcctcctgggttcaagtaattctcctgcctcagcctcccgagtacctgggactacaggcacccaccaccacgctcagctaatttttgtatttttagtagagacggggtttcaccatattggccaggctggtctcgaactcctgaccttgtgatccccccgcctcggccgccc

SEQ ID NO: 162 - DNA, 191 bp, Homo sapiens; identical to SEQ ID NO: 108
  misc_feature: Alu sequence cloned from pk1401_mM-20+++ (see Figure 3)

SEQ ID NO: 163 - DNA, 191 bp, Homo sapiens; identical to SEQ ID NO: 108
  misc_feature: Alu sequence cloned from pk1401_mM-19+++ (see Figure 3)

SEQ ID NO: 164 - DNA, 191 bp, Homo sapiens; identical to SEQ ID NO: 108
  misc_feature: Alu sequence cloned from pk1401_mM-18+++ (see Figure 3)

SEQ ID NO: 165 - DNA, 191 bp, Homo sapiens
  misc_feature: Alu sequence cloned from pk1401_mM-17+++ (see Figure 3)
gggaggccaaatcagatggatcatctgaggtcaggagttcaagaaccaccttatcaacatgaagaatcctggtctctactaaaactacaaaattagccaggtatcatggcaaatgcttgtcatcctagctactcagaaggctgaggcagaggaatcacttgaacctgtgaggcggaggtttcggtgagctg

SEQ ID NO: 166 - DNA, 193 bp, Homo sapiens
  misc_feature: Alu sequence cloned from pk1401_mM-16+++ (see Figure 3)
cagctcactgcaacctccccctcctgggttcaagcgattctcttgcctcagcctcctgagtagctgggattacaggtgcccaccaccacgcccagttaattttttgtagttttagtacagacgaggttccactgtgctgatcaggctagtctcgaactcctgacctcaggtgatccacctgccttggcatctc

SEQ ID NO: 167 - DNA, 191 bp, Homo sapiens; identical to SEQ ID NO: 108
  misc_feature: Alu sequence cloned from pk1401_mM-14+++ (see Figure 3)

SEQ ID NO: 168 - DNA, 194 bp, Homo sapiens
  misc_feature: Alu sequence cloned from pk1401_mM-10 (see Figure 3)
cagctgactgcagtcttgacctcgaaggctcaagcgatcctcccacctctcagcctcacaagtagctgggactactactgacacgcctcaccacacccagcattttttttttttggtagaaacagggtttcattatgttgcccagggtggtctcaaactcctgagctcaagtgatcctccccactcggcctccc

SEQ ID NO: 169 - DNA, 191 bp, Homo sapiens; identical to SEQ ID NO: 108
  misc_feature: Alu sequence cloned from pk1401_mM-8 (see Figure 3)

SEQ ID NO: 170 - DNA, 191 bp, Homo sapiens
  misc_feature: Alu sequence cloned from pk1401_mM-7 (see Figure 3)
cagctcaccgaaacctccgcctcacaggttcaagtgattcctctgcctcagccttctgagtagctaggatgacaagcatttgtcatgatacctggctaattttgtatttttagtagagaccaggattcttcatgttgataaggtggttcttgaactcctgacctcagatgatccatctgatttggcctccc

SEQ ID NO: 171 - DNA, 191 bp, Homo sapiens
  misc_feature: Alu sequence cloned from pk1401_mM-6 (see Figure 3)
cagctcaccacaacctccgcctcctgggttccagcgattctcctgcctcggcctcccaagtagctgggattacaggcacgcaccaatacacctggctaattttgtatttttagcagagacagggtttctccatgttggtcaacctggtctgtaactcctgacctcgggtaatcaacccacttcagcctccc

SEQ ID NO: 172 - DNA, 192 bp, Homo sapiens
  misc_feature: Alu sequence cloned from pk1401_mM-5 (see Figure 3)
cagctcactgcaacctccatttcctgggttcaagcgattctcctgcctcagcctccggagtagctgggaccacagacgtgtgccaccatgcctgggtaattttcatattttcagtagaggtggggctttgccacattgtccaggctggtcttgaactcctgacctcaggtgatccgcccgcctcagcctccc

SEQ ID NO: 173 - DNA, 193 bp, Homo sapiens
  misc_feature: Alu sequence cloned from PK1401_mM-4 (see Figure 3)
tggctcactgcaacctccgcctcccaggttcaagcaattctcctgcctcagtctcccgagtagctgggactaccggcgagtgctaccatgcctgcgtaattttttgtacttttagtagagttggagtttcactacgttggccaggctggtctcaaactcctggcctcaagtgatctgccggcctcagcctccc

SEQ ID NO: 174 - DNA, 191 bp, Homo sapiens
  misc_feature: Alu sequence cloned from pk1401_mM-3 (see Figure 3)
cggctcactgcaagctccgcctcccgggtgcacgccattctcctgcctcagcctcccgagtagctgggactacaggcgcccgccaccacgcccggctaattttttgtatttttagtagaggcagggtttcactgtgttagccaggatggtctcgatctcctgacctcgtgatccgcccgcctctgcctccc

SEQ ID NO: 175 - DNA, 208 bp, Homo sapiens
  misc_feature: Alu sequence cloned from pk1401_mM-2 (see Figure 3)
tgattctcctgcctcagcctcccaagtagctgcgattacaggcatccgccaccacacccaactaattttgtatttttagtagagacaggttttctccatgttggtcaggctagtctcgaattcctgacctcaggtgatctgcctgccttggcttcccaaagtgctgggattacaggcgtgagccactgtgcctggccaaagctatttc

SEQ ID NO: 176 - DNA, 542 bp, Homo sapiens
  misc_feature: Alu sequence cloned from pk1401_mM-2 (see Figure 3)
cagctcactgcaacctcacctcccgggttcaagtgattctcctgcctcagcctcccaagtagctgcgattacaggcatccgccaccacacccaactaattttgtatttttagtagagacaggttttctccatgttggtcaggctagtctcgaattcctgacctcaggtgatctgcctgccttggcttcccaaagtgctgggattacaggcgtgagccactgtgcctggccaaagctatttcttttttctttttcctttttttttttttttttgagacggagtctcgctgtgtcccccaggctggagtacaatggcatgatctcggctcactgcaacctctgcctcccaggtttcaagcgattttcctgcctcagcctcccgagtagctgggattacaggcacccaccaccgtgcccagctaatttttgtatctttaatagagatggggtttcaccatcttggccaggctggtcttgaactcctgacctcatgatccacccacctcagtctcccaaactgctgggagtacagaatctgaattc

SEQ ID NO: 177 - DNA, 191 bp, Homo sapiens
  misc_feature: Alu sequence cloned from BDc_m34-4-----BD (see Figure 3)
tggctcactgtaacctccacctcctggattcaagtgattctcctgcctcagcctcccacgtagctgggactacaggcacacgacaccgcacccagctcattttgtatttttagtagagacagggtttcactatgttggccaggctggtctcaaacttctgacctcaggtgatccacccacctcagccttcc

SEQ ID NO: 178 - DNA, 192 bp, Homo sapiens
  misc_feature: Alu sequence cloned from SZb_m37-10+++ (see Figure 3)
cggctcactgcagcctctacctcccatgttcaagccatcctccagtctcagcctctggagtagttgggattacagatgtgtaccacctcgcctggctaatttttgtatttttagtagagatggggttttgccatgttggccaggctgatctcagattcctgatctcaggtgatccacctgccttggcctccc

SEQ ID NO: 179 - DNA, 191 bp, Homo sapiens
  misc_feature: Alu sequence cloned from SZb_m37-9+++ (see Figure 3)
ggctcactgcagcctctacctcccatgttcaagccatcctccagtctcagcctctggagtagttgggattacagatgtgtaccacctcgcctggctaatttttgtatttttagtagagatggggttttgccatgttggccaggctgatctcagattcctgatctcaggtgatccacctgccttggcctccc

SEQ ID NO: 180 - DNA, 192 bp, Homo sapiens; identical to SEQ ID NO: 178
  misc_feature: Alu sequence cloned from SZb_m37-7+++ (see Figure 3)

SEQ ID NO: 181 - DNA, 191 bp, Homo sapiens; identical to SEQ ID NO: 108
  misc_feature: Alu sequence cloned from SZb_m37-5+++ (see Figure 3)

SEQ ID NO: 182 - DNA, 401 bp, Homo sapiens
  misc_feature: Alu sequence cloned from SZb_m37-3+++ (see Figure 3)
cagctatgacctgattacgccaagctctaatacgactcactatagggaaagctcggtaccacgcatgctgcagacgcgttacgtatcggatccagaattcgtgattgccgggacttcgaaccgtctgggctgcctgaaagcttggactaccaggggtaagcggttcaggggcctcattatcaacaggaactgtgatgacatgtactaacaacactgcccaggtcgggtttgatggcaaatgcaggacatacaaaatactaatatggctgcagggctggaatcaatcgaacgtgggagggatccgtctgcctgagccgacaaagctgatgcaagttccaacatgaattcgtcgacaagcttctcgagcctaggctagctctagaccacacgtgtggggggcc

SEQ ID NO: 183 - DNA, 191 bp, Homo sapiens; identical to SEQ ID NO: 108
  misc_feature: Alu sequence cloned from BDc_m34-10-----BD (see Figure 3)

SEQ ID NO: 184 - DNA, 191 bp, Homo sapiens; identical to SEQ ID NO: 108
  misc_feature: Alu sequence cloned from SZb_m37-2+++ (see Figure 3)

SEQ ID NO: 185 - DNA, 191 bp, Homo sapiens; identical to SEQ ID NO: 177
  misc_feature: Alu sequence cloned from BDc_m34-3-----BD (see Figure 3)

SEQ ID NO: 186 - DNA, 191 bp, Homo sapiens; identical to SEQ ID NO: 108
  misc_feature: Alu sequence cloned from BDc_m34-1-----BD (see Figure 3)

SEQ ID NO: 187 - DNA, 191 bp, Homo sapiens; identical to SEQ ID NO: 108
  misc_feature: Alu sequence cloned from pk211201_M39-2-----BD (see Figure 3)

SEQ ID NO: 188 - DNA, 192 bp, Homo sapiens
  misc_feature: Alu sequence cloned from CtrlC_m57-2 (see Figure 3)
tggctcactgcaacctccacctcccgggttcaagcaattctcgtgcctcagccacctgagtagctgggattataggtgtgcgccaccacacccggctaatttttaaattttttgtagagacggggtttcaccctgttggccaggctggcctcgaactcctaatctcaggtgatctgcccaccttggcctccc

SEQ ID NO: 189 - DNA, 202 bp, Homo sapiens
  misc_feature: Alu sequence cloned from BDd_m43-19-----BD (see Figure 3)
cagctgactgcaacctccacttcccaggttcaagcgattctcctgcctcagcctcctgagtagctggaactagaagcgtgcaccaccacatcccgctaattgtgtgtgtgtgtgtgtgtttgtttagtaaagggggggtttcaccatgttggtcaggctggtctcgaactcctgacaggtgatccacccgccttggcctccc

SEQ ID NO: 190 - DNA, 191 bp, Homo sapiens; identical to SEQ ID NO: 108
  misc_feature: Alu sequence cloned from SZc_m37-26+++ (see Figure 3)

SEQ ID NO: 191 - DNA, 192 bp, Homo sapiens; identical to SEQ ID NO: 118
  misc_feature: Alu sequence cloned from BDd_m34-19-----BD (see Figure 3)

SEQ ID NO: 192 - DNA, 191 bp, Homo sapiens
  misc_feature: Alu sequence cloned from BDd_m34-14-----BD (see Figure 3)
cagcccagtgcaagctccgcctcccaggttcacgtcattctcctgcctcagcctcccgagtagctgggactacaggcgcccgccaccacgcccagctaattttttgtatttttagtagagacaaggtttcaccgtattagccgggatggtcgctatctcctgacctcgtgatctgcccgcctcggcctctc

SEQ ID NO: 193 - DNA, 192 bp, Homo sapiens
  misc_feature: Alu sequence cloned from BDd_m43-14-----BD;DNA (see Figure 3)
ctctgctcactgcagcttctgcctcccgggttcaagtgattctcctgcctcagcctcctgagtagctgggactacaggcatgcaccaccacacccagctaatttttgtatttttagtagagacggggtttcaccatgttggccaggatggtctctatctcttgacctcatgatccgcccgcctcagccttcc

SEQ ID NO: 194 - DNA, 191 bp, Homo sapiens; identical to SEQ ID NO: 108
  misc_feature: Alu sequence cloned from SZc_m37-15+++ (see Figure 3)

SEQ ID NO: 195 - DNA, 190 bp, Homo sapiens
  misc_feature: Alu sequence cloned from SZc_m37-10+++ (see Figure 3)
cagctcactgcaggctccgcctcccgggttcacgccattctcctgcctcagcctccgcagtagctgggactacaggcgcccaccaccatgcccagctaatttttgtatttttagcaaagacagggtttcaccatgttagccaggatggtctcgatctcctgacctcatgatccacctgcctcggcctccc

SEQ ID NO: 196 - DNA, 191 bp, Homo sapiens; identical to SEQ ID NO: 108
  misc_feature: Alu sequence cloned from SZc_m37-7+++ (see Figure 3)

SEQ ID NO: 197 - DNA, 191 bp, Homo sapiens; identical to SEQ ID NO: 108
  misc_feature: Alu sequence cloned from SZc_m37-5+++ (see Figure 3)

SEQ ID NO: 198 - DNA, 190 bp, Homo sapiens
  misc_feature: Alu sequence cloned from SZc_m37-3+++ (see Figure 3)
cagctcactgcaggctccgcctcccgggttcacgccattttcctgcctcagcctccccagtagctgggactacaggcgcccatcaccatgcccagctaatttttgtatttttagcaaagacagggtttcaccatgttagccaggatggtctcgatctcctgacctcctgatccacctgcctcggcctccc

SEQ ID NO: 199 - DNA, 191 bp, Homo sapiens
  misc_feature: Alu sequence cloned from pk0301_M39-14-----BD (see Figure 3)
aagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctccc

SEQ ID NO: 200 - DNA, 191 bp, Homo sapiens; identical to SEQ ID NO: 108
  misc_feature: Alu sequence cloned from PK0301_M37-14+++ (see Figure 3)

SEQ ID NO: 201 - DNA, 190 bp, Homo sapiens
  misc_feature: Alu sequence cloned from PK0301_M37-11+++ (see Figure 3)
cagctcaccgaaacctccgcctcacaggttcaagtgattcctatgccttagccttctgagtagctaggatgacaagcatttgccatgatacctggctaattttgtatttttagtagagaccaggattcttcatgttgataaggcggttcttgaactcctgacctcacatgatccatttgatttggcctcc

SEQ ID NO: 202 - DNA, 190 bp, Homo sapiens
  misc_feature: Alu sequence cloned from RevCompSZB_M37-6+++ (see Figure 3)
cagctcactggcagtctcaatcttccaagttcaaggtgattatcccatctcagcctcccgagtagctgaaactacaggtgcatactaccacgcctagctaatttttttttgtagagatggggttttggccatgttgcccaggctgctctcgaacttctgggcacaagtggtccacccaccttggcctccc

SEQ ID NO: 203 - DNA, 191 bp, Homo sapiens
  misc_feature: Alu sequence cloned from RevCompPK1401_mM-17+++ (see Figure 3)
cagctcaccgaaacctccgcctcacaggttcaagtgattcctctgcctcagccttctgagtagctaggatgacaagcatttgccatgatacctggctaattttgtagttttagtagagaccaggattcttcatgttgataaggtggttcttgaactcctgacctcagatgatccatctgatttggcctccc

SEQ ID NO: 204 - DNA, 191 bp, Homo sapiens; identical to SEQ ID NO: 108
  misc_feature: Alu sequence cloned from RevCompPK1601mM-33+++ (see Figure 3)

SEQ ID NO: 205 - DNA, 191 bp, Homo sapiens; identical to SEQ ID NO: 108
  misc_feature: Alu sequence cloned from RevCompPK1601mM-39+++ (see Figure 3)

SEQ ID NO: 206 - DNA, 426 bp, Homo sapiens
  misc_feature: Alu sequence cloned from CUTPK1601_mM-1_m57-6 (see Figure 3)
gaaccaccattacgccaactctaatacgactcactatagggaaagctcggtaccacgcatgctgcagacgcgttacgtatcggatccagaattcgggattggagggtgtttgcacaatctcagctcactgcaggctccgcctcccgggttcacgccattctcctgcctcagcctcccgagtagctgggactacaggcgcccaccaccatgcccagctaatttttgtatttttagcagagacggggtttcaccatgttggccaggatggtctccaaactcctgacctcctgagacacctgtgtcggggtcccaaactgtgggagtacaggcaactctgaatttttggacaagactcttcgagcctatgctactatctacaccacaccgcgtgggggccccagctcgcggccgctgtattatataata

SEQ ID NO: 207 - DNA, 419 bp, Homo sapiens
  misc_feature: Alu sequence cloned from CUTPK1601mM-57+++ (see Figure 3)
catctatgacatgattgccccgattctccaagctctaattctactgaatgttcggaacgctccatccacgcatgccgtaaacgctttactcctcggttccagaatgcgggattgcctgtacttccatcagttagggaggccaaatcctacggatcatatgaggctatgagaccaagacccaccttatcaacatgaagaatcctggtctctactaaaaatacaatattagccaggtttcatggtatatgcttgtaatcctagctactcacaaggctgaggcagaggaattacttgaacctgtgaggcggaggtttcggtgagctgagattgtccaaacaccctccaatctgaattcgttgacaagcttttcgagcctaggctagctctagaccacacgtgtgggggcccgagctcgcggt

SEQ ID NO: 208 - DNA, 380 bp, Homo sapiens; identical to SEQ ID NO: 142
  misc_feature: Alu sequence cloned from CUTPK1601mM-55+++ (see Figure 3)

SEQ ID NO: 209 - DNA, 192 bp, Homo sapiens
  misc_feature: Alu sequence cloned from utPK1601mM-39+++ (see Figure 3)
gggaggccaaatcagatggatcatctgaggtcaggagttcaagaaccaccttatcaacatgaagaatcctggtctctactaaaaatacaaaattagccaggtatcatggcaaatgcttgtcatcctagctactcagaaggctgaggcagaggaatcacttgaacctgtgaggcggaggtttcggtgagctga

SEQ ID NO: 210 - DNA, 211 bp, Homo sapiens
  misc_feature: Alu sequence cloned from CutPK1601mM-37+++ (see Figure 3)
gggagggtgttttgcacaatctcagctcaccgcaacctttgcctcacgggctcaagtgattctcatgcttgatcctaccaagtagctgggattacaggcacatgccatcatgctgagctaactttggtatttttggtagagacgaggtttcaccatgttggccaggctgtctcaaactcctgacctcagatgatccgtccacctcagcctc

SEQ ID NO: 211 - DNA, 193 bp, Homo sapiens
  misc_feature: Alu sequence cloned from CutPK1601mM-33+++ (see Figure 3)
tgggaggccaaatcagatggatcatctgaggtcaggagttcaagaaccaccttatcaacatgaagaatcctggtctctactaaaaatacaaaattagccaggtatcatggcaaatgcttgtcatcctagctactcagaaggctgaggcagaggaatcacttgaacctgtgaggcggaggtttcggtgagctga

SEQ ID NO: 212 - DNA, 141 bp, Homo sapiens
  misc_feature: Alu sequence cloned from CutPK1601_mM-31+++ (see Figure 3)
tcagcttactgcaacctttgcttcccagtttcaagtgattctcctgtctcatgctccagagaacccggtactacaggcacacgccaccatgctcggctaataatttatgttcttagaatagagattggttttcaccgattt

SEQ ID NO: 213 - DNA, 193 bp, Homo sapiens
  misc_feature: Alu sequence cloned from CutPK1401_mM-17+++ (see Figure 3)
tgggaggccaaatcagatggatcatctgaggtcaggagttcaagaaccaccttatcaacatgaagaatcctggtctctactaaaactacaaaattagccaggtatcatggcaaatgcttgtcatcctagctactcagaaggctgaggcagaggaatcacttgaacctgtgaggcggaggtttcggtgagctga

SEQ ID NO: 214 - DNA, 221 bp, Homo sapiens
  misc_feature: Alu sequence cloned from CutPK1401_mM-2_1+++ (see Figure 3)
tcagctcactgcaacctcacctcccgggttcaagtgattctcctgcctcagcctcccaagtagctgcgattacaggcatccgccaccacacccaactaattttgtatttttagtagagacaggttttctccatgttggtcaggctagtctcgaattcctgacctcaggtgatctgcctgccttggcttcccaaagtgctgggattacaggcgtgagccact

SEQ ID NO: 215 - DNA, 239 bp, Homo sapiens
  misc_feature: Alu sequence cloned from CutPK1401_mM-2_2+++ (see Figure 3)
gagacggagtctcgctgtgtcccccaggctggagtacaatggcatgatctcggctcactgcaacctctgcctcccaggtttcaagcgattttcctgcctcagcctcccgagtagctgggattacaggcacccaccaccgtgcccagctaatttttgtatctttaatagagatggggtttcaccatcttggccaggctggtcttgaactcctgacctcatgatccacccacctcagtctc

SEQ ID NO: 216 - DNA, 192 bp, Homo sapiens
  misc_feature: Alu sequence cloned from CutSZB_M37-6+++ (see Figure 3)
tgggaggccaaggtgggtggaccacttgtgcccagaagttcgagagcagcctgggcaacatggccaaaaccccatctctacaaaaaaaaattagctaggcgtggtagtatgcacctgtagtttcagctactcgggaggctgagatgggataatcaccttgaacttggaagattgagactgccagtgagctga

SEQ ID NO: 217 - DNA, 189 bp, Homo sapiens
  misc_feature: Alu sequence cloned from CutSZB_M37-3+++ (see Figure 3)
tgccgggacttcgaaccgtctgggctgcctgaaagcttggactaccaggggtaagcggttcaggggcctcattatcaacaggaactgtgatgacatgtactaacaacactgcccaggtcgggtttgatggcaaatgcaggacatacaaaatactaatatggctgcagggctggaatcaatcgaacgtgg

SEQ ID NO: 218 - DNA, 390 bp, Homo sapiens
  misc_feature: Alu sequence cloned from PK37-9RfWithM13R (see Figure 3)
gcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttggggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggccctctagatgcatgctcgagcggccgccagtgtgatggatatctgcagaattcggcttgcctgtactcccagcagttt

SEQ ID NO: 219 - DNA, 310 bp, Homo sapiens
  misc_feature: Alu sequence cloned from PK39-4RfWithM13R (see Figure 3)
ccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggccctctagatgcatgctcgagcggccgccagtgtgatggatatctgcagaattcggcttgcctgtactcccagcagttt

SEQ ID NO: 220 - DNA, 250 bp, Homo sapiens
  misc_feature: Alu sequence cloned from PK37-9RrWithM13R (see Figure 3)
gcctgtactcccagcagtttgagaggccaagatgggtggatcacttgaggtctagagctcaagaccagcctggcgacatggtgaaaccccatctctactaaaaatataaaaatcagccaggtgtggtggtgggcacctgtaaccccagctactcaggaggctgaggaagccgaattccagcacactggcggccgttactagtggatccgagctcggtaccaagcttggcgtaatcatggtcatagctgtt

SEQ ID NO: 221 - DNA, 310 bp, Homo sapiens
  misc_feature: Alu sequence cloned from PK39-4RrWithM13R (see Figure 3)
gcctgtactcccagcagtttgagaggccaaatcagatggatcatctgaggtcaggagttcaagaaccaccttatcaacatgaagaatcctggtctctactaaaaatacaaaattagccaggtatcatggcaaatgcttgtcatcctagctactcagaaggctgaggcagaggaatcacttgaacctgtgaggcggaggtttcggtgagctgagattgtgcaaacaccaagccgaattccagcacactggcggccgttactagtggatccgagctcggtaccaagcttggcgtaatcaggtcatagctgtt

SEQ ID NO: 222 - DNA, 549 bp, Homo sapiens
  misc_feature: Alu sequence cloned from PK34-6rwithM13R (see Figure 3)
gcctgtactcccagcagttttgagaggtcaaggaaggaggatcagttgagtccgggagtttgagatgagcctgggcaacatggcaaaacctcgtctctacaaaaaatacaaaaaaagtaagccgggcatggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatca

SEQ ID NO: 223 - DNA, 604 bp, Homo sapiens
  misc_feature: Alu sequence cloned from PK37-1withM13R (see Figure 3)
aacagctatgacctgattacgccaagcttggtaccgagctcggatccactagtaacggccgccagtgtgctggaattcggcttgcctgtactcccagcagtttgggaggccaaatcagatggatcatctgaggtcaggagttcaagaaccaccttatcaacatgaagaatcctggtctctactaaaaatacaaaattagccaggtatcatggcaaatgcttgtcatcctagctactcagaaggctgaggcagaggaatcacttgaacctgtgaggcggaggtttcggtgagctgagattgtgcaaacaccctccaagccgaattctgcagatatccatcacactggcggccgctcgagcatgcatctagagggcccaattcgccctatagtgagtcgtattacaattcactggccgtcgttttacaacgtcgtgactgggaaaaccctggcgttcccaacttaatcgccttgcagcacatccccctttcgcagctggcgtaatagcgaagaggcccgcaccgatcgcccttcccaacagttgcgcagcctgaatggcgaatggacgcgccctgtagcggcgcattaagcgcggcgggtgtggtg

SEQ ID NO: 224 - DNA, 521 bp, Homo sapiens
  misc_feature: Alu sequence cloned from PK37-1rwithM13R (see Figure 3)
gcctgtactcccagcagtttgggaggccaaatcagatggatcatctgaggtcaggagttcaagaaccaccttatcaacatgaagaatcctggtctctactaaaaatacaaaattagccaggtatcatggcaaatgcttgtcatcctagctactcagaaggctgaggcagaggaatcacttgaacctgtgaggcggaggtttcggtgagctgagattgtgcaaacaccctccaagccgaattctgcagatatccatcacactggcggccgctcgagcatgcatctagagggcccaattcgccctatagtgagtcgtattacaattcactggccgtcgttttacaacgtcgtgactgggaaaaccctggcgttcccaacttaatcgccttgcagcacatccccctttcgcagctggcgtaatagcgaagaggcccgcaccgatcgcccttcccaacagttgcgcagcctgaatggcgaatggacgcgccctgtagcggcgcattaagcgcggcgggtgtggtg

SEQ ID NO: 225 - DNA, 531 bp, Homo sapiens
  misc_feature: Alu sequence cloned from PK34-2withM13R (see Figure 3)
gcctgtactcccagcagtttgggaggccgaggcgggcagattgcctgagctcaggagttcgaaaccagcctggacaacacggtgaaaccctgtctctactaaaaatacaaaaaattagccagacgtggtggtgcatgcctgtagtcccagctagtcaggaggctgaggcaggagaatcacttgaacccagcaggaaaaggttgtggtgagctgagattgtgcaaacaccctccaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacattcccctttcgccagctggcgtaatagctaagaggcccgcaccgatcgtcccttcccaacagttgcgcagcctgaatggcgaatggacgcgccctgtagcggcgcattaagcgcggcgggtgtggtggttac

SEQ ID NO: 226 - DNA, 346 bp, Homo sapiens
  misc_feature: Alu sequence cloned from PK34-7withM13R (see Figure 3)
ggagggtgtttgcacaatctcggcttactgcaacctccactcctgggcttaaacggtcctcccacctcatcttcccgagtagcagggtccacaggtgcacaccaccatgcctggctatattttttttttttttggatttttgataaagacaggatgtcaacatgttgcccacgctggtcttcaaccccttgaactcaaattcatctgcttctgcctcccaaactggtgggagtcttgaggtgggcgaaccacctgatgttacgaatatgagacttttcggcctgattccggccaaactctcgtcttattttttataatctaataaatcccatctaggggctagggt

SEQ ID NO: 227 - DNA, 399 bp, Homo sapiens
  misc_feature (49)..(49): n is a, g, c, or t
ggagggtgtttgcacaatctcagctcaccgaaacctccgcctcacaggntcaagtgattcctctgcctcagccttctgagtagctaggatgacaagcatttgccatgatacctggctaattttgtacttttagtagagaccaggattcttcatgttgataaggtggttcttgaactcctgacctcagatgatccatctgatttggcctcccaaactgctgggagtacaggcaagccgaattctgcagatatccatcacactggcggccgctcgagcatgcatctagagggcccaattcgccctatagtgagtcgtattacaattcactggccggcgttttacaacgtcgtgactgggaaaaccctggcgttacccaacttaatcgccttgcagcacatc

SEQ ID NO: 228 - DNA, 429 bp, Homo sapiens
  misc_feature: Alu sequence cloned from PK34-9withM13R (see Figure 3)
gcctgtactcccagcagtttgggaggtcaaggtggagagatcacttgaggtcaggagttcgagaccagcctaaccaatatgatgaaaccccatctctactaaaaatacaaaaattagccgggcgtggtggtgcgcacctgtaatcccagctactcaggaggctgaggcaggagaattgcttgaaccagggagtcggaggttgcagtaagccaagattgtgcaaacaccctccaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaa

SEQ ID NO: 229 - DNA, 357 bp, Homo sapiens
  misc_feature: Alu sequence cloned from PK37-3.1withM13R (see Figure 3)
cctgtactcccagcagtttggaagtggatcacttgaggccagggactcaagaccaacctggccaatatggcaaaacccggctaaaaatacaaaaattagctggacatggttgcaggtgcctgtaatcccagctactcgggaggttgtggcatgagaatcacttgaacctgggaggcagaggctgcagcgagcagagattgtgcaaacaccctaagccgaattctgcagatatccatcacactggcggccgctcgagcatgcatctagagggcccaattcgcccctatagtgagtcgcattacaatttactggcccgtcgttttacaaccgtcccgactgggaaaaccctggcgttac

SEQ ID NO: 230 - DNA, 517 bp, Homo sapiens
  misc_feature: Alu sequence cloned from PK37-7withM13R (see Figure 3)
gcctgtactcccagcagtttgggaggccaaatcagatggatcatctgaggtcaggagttcaagaaccaccttatcaacatgaagaatcctggtctctactaaaaatacaaaattagccaggtatcatggcaaatgcttgtcatcctagctactcagaaggctgaggcagaggaatcacttgaacctgtgaggcggaggtttcggtgagctgagattgtgcaaacaccctccaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggt

SEQ ID NO: 231 - DNA, 566 bp, Homo sapiens
  misc_feature: Alu sequence cloned from PK39-2withM13R (see Figure 3)
gcctgtactcccagcagtttgggaggctgaggcggttggatcacaaggttaggagtttgaggccagcctggccaataagatgaaaccccatctgtactaaaaatacaaaaattagccaaacgtggtggtgggcacctgtagtcccagctacttgggaggctgaggcaaaaaaattgcttgaacctgggaggcggaggttgcagcgagctgagattgtgcaaacaccctccaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttccaacagttgcgcagcctgaatggcgaatggacgcgccctgtagcggcgcattaagccccggcgggtgtggtggttacgcgcagcgtgaccgctacacttgccagcgccctagcgcc

SEQ ID NO: 232 - DNA, 522 bp, Homo sapiens
  misc_feature: Alu sequence cloned from BD43-13 (see Figure 3)
gcctgtactcccagcagtttgggaggccgaggtgggcggatggcctgaagccaggagtttgagactagcctggcctacatggtgaaaacctgtctctactaaaaatacaataattagccggacatggtgacacctataataccagctactcgggaagctgagccatgagaattgcttgaacccggaaggtggaggttgcagtgagctgagattgtgcaaacaccctccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattccctttttgcggcattttgccttcctgttttgctcacccacaaaccctggtgaaagtaaaagatgctgaagatcagttgg

SEQ ID NO: 233 - DNA, 374 bp, Homo sapiens
  misc_feature: Alu sequence cloned from BD43-18withM13R (see Figure 3)
gcctgtactcccagcagtttgggaggccaaagcggacggatcatatgaggtcgagagttcaagaaccatgttatcaatgtgaaaaatctgggtctatactaaaaacacaaatttacccagggttgatggaagatgctggtcatcctaattcctcagaaggctgaggcagaggaatcatttgaacctgggaggcggacgttcaggggacctgaaatggggcaaccaccttcaaagccgaattttgcaaatttccataacatggggggcgcgttcaaccttgcttttaaagggcccatttcccttatatggagtcgatttacaattaacgggcggtcgttttacacctttggatgggaaaaaccctgcgtacccca

SEQ ID NO: 234 - DNA, 499 bp, Homo sapiens
  misc_feature: Alu sequence cloned from Ctrlm57-7withM13R (see Figure 3)
acaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcaggatctcgtcgtgaccatggcgatgcctgcttgcca

SEQ ID NO: 235 - DNA, 396 bp, Homo sapiens
  misc_feature: Alu sequence cloned from pk50-26withM13R(-46) (see Figure 3)
ttaaaaccgaaatgccatgatacgccaagcttggtaccgagctacggacccactagctaacggccgccagtgtgcctgacctcttatccctgcacgatatccactcacactgctggctgtccgtgcatgcatctaccgggctcaattcgccctatagtgagtcggattacaattcactggccgtcgttttacaacgtcgtgactgggaaaaccctggtgttacccaacttaatcgccttgcagcacatccccctttcgccagcttggcgcaatagcgaagaggcatcgctccgatcgccctttccaacagcttgcgcagccagaatggctaatggacgcgccctgtctccggccgcattaatccgcggcgggtgtggcggttaccccgcagcagtg

SEQ ID NO: 236 - DNA, 468 bp, Homo sapiens
  misc_feature: Alu sequence cloned from PK34-1withM13R (see Figure 3)
ggagggtgtttgcacaatctggagggtgtttgcacaatctcggctcaccacaacctctacctcccaggttcaagcaattctgcctcagcctcccaagtagctgggactacaggcgtgcaccaccacacctggctaatttctgtatttttagtagaaacagggtttcaccatgttggccaggctggtctcgaactcctgaccttgtgatccgcctaccttggctttccaaactgctgggagtacaggcaagccgaattctgcagatatccatcacactggcggccgctcgagcatgcatctagagggcccaatccgccctatagtgagtcgtattacaatccactggccgaagtttacaacggcgtgactgggaaaaccctggcgttacccaacttaatcgccttgcagcacatccccctttcgccagctggcgaaatagcgaagaggcccgcaccgatcgcccttccc

SEQ ID NO: 237 - DNA, 517 bp, Homo sapiens
  misc_feature: Alu sequence cloned from PK34-3withM13R (see Figure 3)
ggagggtgtttgcacaatctctgctcactacaacttctacctcccaggctcaagcaatcctcccatgtagctgggaccacaggtgtgcaccaccatgccaagctaatttttgtatttttttgtagagtgaggtttcaccatattgcccaggttggtcttgaactcctaagctcaagcaatccacctgcctcagcttctcaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaaaaggcccgcaccgatcgcccttcccaacagttgcgcagcctgaatggcgaatggacgcgccctgtagcggcgcattaagcgcggcgggtgtggtggttacg

SEQ ID NO: 238 - DNA, 529 bp, Homo sapiens
  misc_feature: Alu sequence cloned from PK34-4withM13R (see Figure 3)
ggagggtgtttgcacaatctcggctcatggcacccctcgcctcccagattcaaatgatactcctgcctcagcctcctgagtagctgggattacatgcatgcgccaccatgcccagctaattttttgtatttttagtagagacggggtttcaccatgttggccagactagacttgaactcctgacctcgtgatccacccacctcaacctcccaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaaaaggcccgcaccgatcgcccttcccaacagttgcgcagcctgaatggcgaatggacgcgccctgtagcggcgcattaagcgcggcgggtgtggtggttacg

SEQ ID NO: 239 - DNA, 436 bp, Homo sapiens
  misc_feature: Alu sequence cloned from PK34-5withM13R (see Figure 3)
ggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtgggtcttgaactcctgacctcagatgatccatctgatttggcctcccaaactgctgggagtacaggcaagccgaattctgcaaatatccatcacactggcggccgttcgagcatgcatctaaagggcccaattcgccctataggtgagtcgtattacaattcactggccgtcgttttacaacgtcgtgactgggaaaaccctggcgttacccaacttaatcgccttgcagcacatccccctttcgccagctggcgtaatagcgaagaggccc

SEQ ID NO: 240 - DNA, 521 bp, Homo sapiens; identical to SEQ ID NO: 224
  misc_feature: Alu sequence cloned from PK37-1withM13R (see Figure 3)

SEQ ID NO: 241 - DNA, 482 bp, Homo sapiens
  misc_feature: Alu sequence cloned from PK37-2withM13R (see Figure 3)
ggagggtgtttgcacaatctcagctcattgcaacttccagctcccaggttcaagcgattctccttcctcagcctcccaagtagttgggattacaggcatgcaccatcatgcccggctaatttttgtatttttagtagagacagggtttcaccatattggccaggctggtcttgaactcctgacctcgtgttccacccacctcagcctcccaaactgctgggagtacaggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacattccctttcgccagctggcgtaatagcgaagaggcccgcaccgatcgcccttcccaacagttgcgcagcctgaatggcgaatggacgcgcc

SEQ ID NO: 242 - DNA, 525 bp, Homo sapiens
  misc_feature: Alu sequence cloned from PK37-4withM13R (see Figure 3)
ggagggtgtttgcacaatctcagctcattgcaacctcccaggttcaagcgattctcctgcctcagcctcctgagtagctgggatcacaggtgtgtgccaccattcctggctaatttttgtatttctagtagagatggggttttaccatgttggtcaggctggtctcaaactcctgacctcatgatctgcccaccttggcctcccaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgccctttcccaacagttgcgcagcctgaatggcgaatggacgcgccctgtagtcggcgcattaagcgcggcgggtgtggtggttacgc

SEQ ID NO: 243 - DNA, 465 bp, Homo sapiens
  misc_feature: Alu sequence cloned from PK37-5withM13R (see Figure 3)
ggagggtgtttgcacaatctcagctcactacaacctctgcctcccaggttcaagcgattctcatgcctcggcttctcaagttgctgggactacgggcacacgccagcacggctggctaatttttgtatttttagtagagacagggtttcaccgtcttggccatgctggtctcaaactcctgacctcatgatccacccgccttggcctcccaaactgctgggagtacaggcaagccgaattctgcagatatccatcacactggcggccgctcgagcatgcatctagagggcccaattcgccctatagtgagtcgtattacaatttactggccgtcgttttacaacgtcgtgactgggaaaacccctggcgttacccaacttaatcgccttgcagcacatccccctttcgccagctggcgtaatagcgaagaggcccgcaccgatcgcccttcccaacagttgcgcc

SEQ ID NO: 244 - DNA, 531 bp, Homo sapiens
  misc_feature: Alu sequence cloned from PK37-6withM13R (see Figure 3)
ggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcg

SEQ ID NO: 245 - DNA, 517 bp, Homo sapiens
  misc_feature: Alu sequence cloned from PK37-8withM13R (see Figure 3)
ggagggtgtttgcacaatctttgctcactgcaatctccacctcccgggttcaagtgattctcctgcctcagactgctgaatacttgggattacaggcacccgccaccacaccttgctaattttttggatttttaatagagatgggggttcaccatgtcaaccaggctggtcttgaactcctgaccttaggtgatccacccacctcagcctcccaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgg

SEQ ID NO: 246 - DNA, 620 bp, Homo sapiens
  misc_feature: Alu sequence cloned from PK37-9withM13R (see Figure 3)
aacagctatgaccatgattacgccaagcttggtaccgagctcggatccactagtaacggccgccagtgtgctggaattcggcttcctcagcctcctgagtagctggggttacaggtgcccaccaccacacctggctgatttttatatttttagtagagatggggtttcaccatgtcgccaggctggtcttgagctctagacctcaagtgatccacccatcttggcctctcaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttccccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgc

SEQ ID NO: 247 - DNA, 394 bp, Homo sapiens
  misc_feature: Alu sequence cloned from PK37-26withM13R (see Figure 3)
ggagggtgtttgcacaatctcggctcacagtagcctctgcctcctgggttcaagcgattctcctgcctcagcctcccgagtagctgggattacaggcatgcgccaccatgtccatctaattttgtatttttagtagagatggggtttctccatgttggtcaggctggtctcgaactcccaacctcaggtgatccacccgccttggcctcccaaactgctgggagtacaggcaagccgaattctgcagatatccatcacactggcggccgctcgagcatgcatctagagggcccaattcgccctatagtgagtcgtattacaattcactggccgtcgttttacaacgtcgcgactgggaaaaccctgtcgttacccaactcaatcgccttgcagc

SEQ ID NO: 248 - DNA, 566 bp, Homo sapiens
  misc_feature: Alu sequence cloned from PK39-3withM13R (see Figure 3)
ggagggtgtttgcacaatcttggctcactgcaacctctgcctcctgggcccaagccatcttcctacctcagcttcccgagtagctggactacaggtgtgagccatcacgcccagccaatttttgtatttttagtagagacgaggtttcaccatgttggcctggctggccttgatctcctgacctagtgatctccccgcctcagcctctcaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttccaacagttgcgcagcctgaatggcgaatggacgcgccctgtagcggcgcattaaacgcggcgggtgtggtggttacgcgcagcgtgaccgctacacttgccagcgccctagcgcccg

SEQ ID NO: 249 - DNA, 600 bp, Homo sapiens
  misc_feature: Alu sequence cloned from PK39-4withM13R (see Figure 3)
aacagctatgacctgattacgccaagcttggtaccgagctcggatccactagtaacggccgccagtgtgctggaattcggcttggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctctcaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtgg

SEQ ID NO: 250 - DNA, 527 bp, Homo sapiens
  misc_feature: Alu sequence cloned from PK39-6withM13R (see Figure 3)
ggagggtgtttgcacaatctcagctcaccgaaacctccgcctcacaggttcaagtgattcctctgcctcagccttctgagtagctaggatgacaagcatttgccatgatacctggctaattttgtatttttagtagagatggggttttgccatgttggccaggctggtctcaaactcctgacctcaagtgatcccccacctcggcctcccaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttac

SEQ ID NO: 251 - DNA, 526 bp, Homo sapiens
  misc_feature: Alu sequence cloned from PK39-7withM13R (see Figure 3)
ggagggtgtttgcacaatctggagggtgtttgcacaatctcggctcaccacaatctttgcctttcgggttcaagggattctcctgcctcagcctcccgagtagctgggattacaggcatgtgccaccacacccggctaatgttgtagttttagtagagacggggtttctctatgttggttaggctggtctcaaactcctgacctcaggtgatctacccgcctcggcctctcaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagccctgaatggcgaatggacgcgccctgtagcggcgcatta

SEQ ID NO: 252 - DNA, 491 bp, Homo sapiens
  misc_feature: Alu sequence cloned from PK39-8withM13R (see Figure 3)
ggagggtgtttgcacaatctcagctcaccgaaacctccgcctcacaggttcaagtgattcctctgcctcagccttctgagtagctaggatgacaagcatttgccatgatacctggctaattttgtatttttagtagagatggggttttgccatgttggccaggctggtctcaaactcctgacctcaagtgatcccccacctcggcctcccaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctg

SEQ ID NO: 253 - DNA, 539 bp, Homo sapiens
  misc_feature: Alu sequence cloned from PK39-9withM13R (see Figure 3)
ggagggtgtttgcacaatctcagctcattgcaacctccacctcccgggttcaagcaattcccctgcctcagcctcctgagtagctggaactacaggcacgcgccaccacgtctggttaatttttttgtatttttatagagatggggttttaccatgttgcccaggctggtcttaaactcctgggctcaagctatccactcgccttggcctcccaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttccaacagttgcgcagcctgaatggcgaatggacgcgccctgtagcggcgcattaagcgcggcgggtgtggtggttacgcgcagcgtg

SEQ ID NO: 254 - DNA, 541 bp, Homo sapiens
  misc_feature: Alu sequence cloned from PK39-10withM13R (see Figure 3)
ggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatttgatttggcctcccaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgacc

SEQ ID NO: 255 - DNA, 327 bp, Homo sapiens
  misc_feature: Alu sequence cloned from PK39-12withM13R (see Figure 3)
ggagggtgtttgcacaatcttggctcactgcaacttttgcctcctgggttcaagcaattctcctgcctcagcctcccgagtagctgggactataggcacgcgccatcacgccgggttattttgtatttttagtacagacggggtgtttacatggtggtcaagctgggtttgaacttctgacctcaagtgatcctgcccgcctcggctttccaaactgctgggagtacatggcaagcccgaattctgcagatatccatcacacctggcggccgctcgagcttgcatctagagggcccaattccgccctattctgagtcgtatctacaa

SEQ ID NO: 256 - DNA, 416 bp, Homo sapiens
  misc_feature: Alu sequence cloned from BD43-1withM13R (see Figure 3)
ggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaagccgaattctgcagatatccatcacactggcggccgctcgagcatgcatctagagggcccaattcgccctatagtgagtccgtattacaattcactggccgtcgttttacaacgtcgtgactgggaaaaccctggcgttacccaacttaatcgccttgcagcacatcccccctttcgcacctg

SEQ ID NO: 257 - DNA, 567 bp, Homo sapiens
  misc_feature: Alu sequence cloned from BD43-2withM13R (see Figure 3)
ggagggtgtttgcacaatctcagctcaccgaaacctccgcctcacaggttcaagtgattcctctgcctcagccttctgagtagctaggatgacaagcatttgccatgatacctggctaattttgtatttttagtagagaccaggattcttcatgttgataaggtggttcttgaactcctgacctcagatgatccatctgatttggcctcccaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcc

SEQ ID NO: 258 - DNA, 545 bp, Homo sapiens
  misc_feature: Alu sequence cloned from BD43-6withM13R (see Figure 3)
ggagggtgtttgcacaatctggggttcaagggaagagtccaggctgcagataaagatttgggagttgtcagtatagcaatttcattgttgttattactgttgttgttttgtagagatagggtctcactatgttgcccacgctggtcttgaactcctgagctcaagcgatcctcctgcttcagcctcccaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttccaacagttgcgcagcctgaatggcgaatggacgcgccctgtagcggcgcattaagcgcggcgggtgtggtggttacgcgcagcgtgaccgctacacttgccagcgccctagcgcccg

SEQ ID NO: 259 - DNA, 531 bp, Homo sapiens
  misc_feature: Alu sequence cloned from BD43-8 (see Figure 3)
ggagggtgtttgcacaatcttggctcactgcaacctccacctcgcagttcaagcaattcttgtgccttagcctcctgaatagtagctgggattacgggcgtgtgccatcacacccagctaatttttgtatttttagtagagacagttgtccaggctggtcttgaattcctggcctcaagagatccgctggctttggcctctcaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgcctgtagcggcgcattaagcgcggcgggtgtggtggttacgcgcagcgtgacc

SEQ ID NO: 260 - DNA, 531 bp, Homo sapiens; identical to SEQ ID NO: 259
  misc_feature: Alu sequence cloned from BD43-8(2)withM13R BD43-8 (178, 100, 11q22.3) (see Figure 3)

SEQ ID NO: 261 - DNA, 529 bp, Homo sapiens
  misc_feature: Alu sequence cloned from BD43-9withM13R (see Figure 3)
ggagggtgtttgcacaatctcagctcactgcaaccttcgcctcccgggttcaagtgattctcctgcctcagcctcctgagtagctaggactatagatgcccccaccacgcctggctaatatttgtatttttttagtacagtcggggttttgccatgttggccaggctgatctcgaacccctgacctcaactgatccacccacctcggccttccaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacattcccctttcgccagctggcgtaatagcgaagaggcccgcaccgatcgcccttccaacagttgcgcagcctgaatggcgaatggacgcgccctgtagcggcgcattaagcccggcgggtgtggtggttac

SEQ ID NO: 262 - DNA, 563 bp, Homo sapiens
  misc_feature: Alu sequence cloned from BD43-10withM13R (see Figure 3)
ggagggtgtttgcacaatctcagctcactgcaacctccctcttctgcattcaaatgattctcatgcctcagccttccgagtagctggaattacagacatgtactaccacaccaggctaagttttgtatttttagtagagacgaggtttcaccatgttggccaggctggtcttgaactcctggcctcaagtgatccacctgccttggcttcccaaactgctgggagtacaggcaagccgaattctgcagatatccatcacactggcggccgctcgagcatgcatctagagggcccaattcgccctatagtgagtcgtattacaattcactggccgtcgttttacaacgtccgtgactgggaaaaccctggcgttacccaacttaatcgccttgcagcacatcccccctttcgccagctggcgtaatagcgaagaggcccgcaccgatcgcccttcccaacagtttgcgcagcctgaatggcgaatggacgcgccctgtagcggcgcattaagcgcggcgggtgtggtggttacgcgcagcgtgaccgctacacttgccagcgcc

SEQ ID NO: 263 - DNA, 566 bp, Homo sapiens
  misc_feature: Alu sequence cloned from BD43-14 (191, 100, 16q24.2) withM13R (see Figure 3)
ggagggtgtttgcacaatctcagctcaccacaaccttttcctgctgggttcaagtgattatcctgcctcaacctcccgactagctgggattacaggcatgcaccaccatgcctggctaattttgtatttttagcagagacagtgtttctccatgttggtgaggctggtctcaaactcccgacctcaggtgatccgcctgcctcagcctcccaaactgctgggagtacaggcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtaacggcgcattaagcgcggcgggtgtggtggttacgcgcagcgtgaccgctacacttgccagcgccctagcgc